SEQUENCE LISTING (9 sequences)

SEQ ID NO: 1 - DNA, 627 bp, Homo sapiens
tctcgaggatccgccgccatggactacaaggacgacgatgataagcacccgatagccctgaccgggttcgagcagaaatgcccaaccagcttcgcaagcaattggaggcgatcatagccactgatccacttaacgacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactaggtcgacc

SEQ ID NO: 2 - protein, 204 aa, Homo sapiens
MDYKDDDDKHPIALPKHQPTPDDRVRAEMPNQLRKQLEAIIATDTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTA

SEQ ID NO: 3 - DNA, 3342 bp, Homo sapiens
atggagctggagaactataaacagcccgtggtgctgagagaggacaactgccgaaggcgccggaggatgaagccgcgcagtgctgccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggacgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcggcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctacccatacgacgtaccaccagactacgcttaaggatcc

SEQ ID NO: 4 - protein, 1110 aa, Homo sapiens
MELENYKQPVVLREDNCRRRRRMKPRSAASLSSMELIPIEFVLPTSQRKCKSPETALLHVAGHGNVEQMKAQVWLRALETSVAADFYHRLGPHHFLLLYQKKGQWYEIYDKYQVVQTLDCLRYWKATHRSPGQIHLVQRHPPSEESQAFQRQLTALIGYDVTDVSNVHDDELEFTRRGLVTPRMAEVASRDPKLYAMHPWVTSKPLPEYLWKKIANNCIFIVIHRSTTSQTIKVSPDDTPGAILQSFFTKMAKKKSLMDIPESQSEQDFVLRVCGRDEYLVGETPIKNFQWVRHCLKNGEEIHVVLDTPPDPALDEVRKEEWPLVDDCTGVTGYHEQLTIHGKDHESVFTVSLWDCDRKFRVKIRGIDIPVLPRNTDLTVFVEANIQHGQQVLCQRRTSPKPFTEEVLWNVWLEFSIKIKDLPKGALLNLQIYCGKAPALSSKASAESPSSESKGKVRLLYYVNLLLIDHRFLLRRGEYVLHMWQISGKGEDQGSFNADKLTSATNPDKENSMSISILLDNYCHPIALPKHQPTPDPEGDRVRAEMPNQLRKQLEAIIATDPLNPLTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTAMLHDFTQQVQVIEMLQKVTLDIKSLSAEKYDVSSQVISQLKQKLENLQNSQLPESFRVPYDPGLKAGALAIEKCKVMASKKKPLWLEFKCADPTALSNETIGIIFKHGDDLRQDMLILQILRIMESIWETESLDLCLLPYGCISTGDKIGMIEIVKDATTIAKIQQSTVGNTGAFKDEVLNHWLKEKSPTEEKFQAAVERFVYSCAGYCVATFVLGIGDRHNDNIMITETGNLFHIDFGHILGNYKSFLGINKERVPFVLTPDFLFVMGTSGKKTSPHFQKFQDICVKAYLALRHHTNLLIILFSMMLMTGMPQLTSKEDIEYIRDALTVGKNEEDAKKYFLDQIEVCRDKGWTVQFNWFLHLVLGIKQGEKHSAYPYDVPDYA

SEQ ID NO: 5 - DNA, 3237 bp, Homo sapiens
atggagctggagaactataaacagcccgtggtgctgagagaggacaactgccgaaggcgccggaggatgaagccgcgcagtgctgccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggacgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcggcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctacccatacgacgtaccaccagactacgcttaaggatcc

SEQ ID NO: 6 - protein, 1076 aa, Homo sapiens
MELENYKQPVVLREDNCRRRRRMKPRSAASLSSMELIPIEFVLPTSQRKCKSPETALLHVAGHGNVEQMKAQVWLRALETSVAADFYHRLGPHHFLLLYQKKGQWYEIYDKYQVVQTLDCLRYWKATHRSPGQIHLVQRHPPSEESQAFQRQLTALIGYDVTDVSNVHDDELEFTRRGLVTPRMAEVASRDPKLYAMHPWVTSKPLPEYLWKKIANNCIFIVIHRSTTSQTIKVSPDDTPGAILQSFFTKMAKKKSLMDIPESQSEQDFVLRVCGRDEYLVGETPIKNFQWVRHCLKNGEEIHVVLDTPPDPALDEVRKEEWPLVDDCTGVTGYHEQLTIHGKDHESVFTVSLWDCDRKFRVKIRGIDIPVLPRNTDLTVFVEANIQHGQQVLCQRRTSPKPFTEEVLWNVWLEFSIKIKDLPKGALLNLQIYCGKAPALSSKASAESPSSESKGKVRLLYYVNLLLIDHRFLLRRGEYVLHMWQISGKGEDQGSFNADKLTSATNPDKENSMSISILLDNYCHPIALPKHQPTPDPEGDRVRAEMPNQLRKQLEAIIATDPLNPLTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTAMLHDFTQQVQVIEMLQKVTLDIKSLSAEKYDVSSQVISQLKQKLENLQNSQLPESFRVPYDPGLKAGALAIEKCKVMASKKKPLWLEFKCADPTALSNETIGIIFKHGDDLRQDMLILQILRIMESIWETESLDLCLLPYGCISTGDKIGMIEIVKDATTIAKIQQSTVGNTGAFKDEVLNHWLKEKSPTEEKFQAAVERFVYSCAGYCVATFVLGIGDRVPFVLTPDFLFVMGTSGKKTSPHFQKFQDICVKAYLALRHHTNLLIIILFSMMLMTGMPQLTSKEDIEYIRDALTVGKNEEDAKKYFLDQIEVCRDKGWTVQFNWFLHLVLGIKQGEKHSAYPYDVPDYA

SEQ ID NO: 7 - DNA, 3207 bp, Homo sapiens
atgcctccaagaccatcatcaggtgaactgtggggcatccacttgatgcccccaagaatcctagtggaatgtttactaccaaatggaatgatagtgactttagaatgcctccgtgaggctacattagtaactataaagcatgaactatttaaagaagcaagaaaataccctctccatcaacttcttcaagatgaatcttcttacattttcgtaagtgttacccaagaagcagaaagggaagaattttttgatgaaacaagacgactttgtgatcttcggctttttcaaccatttttaaaagtaattgaaccagtaggcaaccgtgaagaaaagatcctcaatcgagaaattggttttgctatcggcatgccagtgtgcgaatttgatatggttaaagatcctgaagtacaggacttccgaagaaatattcttaatgtttgtaaagaagctgtggatcttagggatcttaattcacctcatagtagagcaatgtatgtctatccgccacatgtagaatcttcaccagagctgccaaagcacatatataataaattggatagaggccaaataatagtggtgatttgggtaatagtttctccaaataatgacaagcagaagtatactctgaaaatcaaccatgactgtgtgccagaacaagtaattgctgaagcaatcaggaaaaaaactagaagtatgttgctatcatctgaacaattaaaactctgtgttttagaatatcagggcaagtacattttaaaagtgtgtggatgtgatgaatacttcctagaaaaatatcctctgagtcagtataagtatataagaagctgtataatgcttgggaggatgcccaatttgaagatgatggctaaagaaagcctttattctcaactgccaatggactgttttacaatgccatcttattccagacgcatttccacagctacaccatatatgaatggagaaacatctacaaaatccctttgggttataaatagagcactcagaataaaaattctttgtgcaacctacgtgaatctaaatattcgagacattgacaagatttatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaggaaggatgaaacacaaaaggtacagatgaagtttttagttgagcaaatgaggcgaccagatttcatggatgccctacagggcttgctgtctcctctaaaccctgctcatcaactaggaaacctcaggcttaaagagtgtcgaattatgtcttctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

SEQ ID NO: 8 - protein, 1068 aa, Homo sapiens
MPPRPSSGELWGIHLMPPRILVECLLPNGMIVTLECLREATLVTIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPHVESSPELPKHIYNKLDRGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLKMMAKESLYSQLPMDCFTMPSYSRRISTATPYMNGETSTKSLWVINRALRIKILCATYVNLNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQERKDETQKVQMKFLVEQMRRPDFMDALQGLLSPLNPAHQLGNLRLKECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 9 - protein, 195 aa, Homo sapiens
HPIALPKHQPTPDDRVRAEMPNQLRKQLEAIIATDTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTA

(19) United States
(12) Patent Application Publication
(10) Pub. No.: US 2006/0026702 A1
(43) Pub. Date: Feb. 2, 2006

(54) Phosphoinositide 3-kinase mediated inhibition of GPCRs

(75) Inventors: Howard A. Rockman, Chapel Hill, NC (US); Sathyamangla V. Naga Prasad, Durham, NC (US); Stephane A. Laporte, Outremont (CA); Larry S. Barak, Durham, NC (US); Marc G. Caron, Hillsborough, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 10/902,137

(22) Filed: Jul. 30, 2004

Related U.S. Application Data
(63) Continuation of application No. 10/101,235, filed on Mar. 19, 2002, now abandoned.

(51) Int. Cl. G01N 33/53; G01N 33/567; A01K 67/027

(52) U.S. Cl. 800/18; 435/7.2

(57) ABSTRACT

The present invention relates to compounds that alter GPCR internalization and new methods for their identification. Compounds of this invention include modified phosphoinositide 3-kinase (PI3K), modified HEAT domain, modified β-adrenergic receptor kinase 1 (βARK1), as well as other peptides or small molecules that alter GPCR internalization. The present invention also includes the use of such compounds to treat GPCR-related diseases, such as cardiovascular disease, heart failure, asthma, nephrogenic diabetes insipidus, or hypertension.

This work was supported by National Institutes of Health Grants HL56687, HL 61365 and NS 19576, and therefore the government may have certain rights to the invention.

### FIELD OF THE INVENTION

The present invention relates to compounds that alter GPCR internalization and new methods for their identification. Compounds of this invention include modified phosphoinositide 3-kinase (PI3K), modified HEAT domain, modified β-adrenergic receptor kinase 1 (βARK1), as well as other peptides or small molecules that alter GPCR internalization. The present invention also includes the use of such compounds to treat GPCR-related diseases, such as cardiovascular disease, heart failure, asthma, nephrogenic diabetes insipidus, or hypertension.

### BACKGROUND

G protein-coupled receptors (GPCRs) are cell surface proteins that translate hormone or ligand binding into intracellular signals. GPCRs are found in all animals, insects, and plants. GPCR signaling plays a pivotal role in regulating various physiological functions including phototransduction, olfaction, neurotransmission, vascular tone, cardiac output, digestion, pain, and fluid and electrolyte balance. Although they are involved in various physiological functions, GPCRs share a number of common structural features. They contain seven membrane domains bridged by alternating intracellular and extracellular loops and an intracellular carboxyl-terminal tail of variable length.

The magnitude of the physiological responses controlled by GPCRs is linked to the balance between GPCR signaling and signal termination. The signaling of GPCRs is controlled by a family of intracellular proteins called arresting. Arrestins bind activated GPCRs, including those that have been agonist-activated and especially those that have been phosphorylated by G protein-coupled receptor kinases (GRKs). These processes are affected by other molecules such as phosphatidylinositol 3,4,5-triphosphate (Ptdlns (3,4,5) P₃), the main product catalyzed by the lipid kinase activity of phosphoinositide 3-kinase (PI3K).

Approximately fifty percent of the therapeutic drugs in use today target or interact directly with GPCRs. See eg., Jurgen Drews, (2000) “Drug Discovery: A Historical Perspective,” Science 287:1960-1964, which is incorporated by reference herein in its entirety. Although only several hundred human GPCRs are known, it is estimated that several thousand GPCRs exist in the human genome. Of these known GPCRs, many are orphan receptors that have yet to be associated with a function or ligands.

The different processes which affect GPCR signaling, signal termination, internalization, recycling, and the like present targets for screening compounds for potential use in treating GPCR-related diseases and disorders. Accordingly, there is a need to identify these processes, and to develop methods of screening and identifying compounds which affect GPCR signaling and signal termination.

### SUMMARY

The present inventors have identified a target for inhibitors of GPCR desensitization. In accordance therewith, the present invention provides methods for screening for such inhibitors, and methods of treating GPCR-related diseases. The invention also provides pharmaceutical compositions for treating GPCR-related diseases.

The invention relates to methods of screening compounds and sample solutions for the activity of modulating GPCR internalization. Compounds and sample solutions may be screened by a method including providing a cell comprising molecules involved in GPCR internalization, wherein the molecules involved in GPCR internalization comprise (βARK1), (PI3K), GPCR, and arrestin. Preferably, at least one of the molecules is detectably labeled. The sample compounds or sample solutions are provided and the cells are exposed to the sample compounds or solutions. The location in the cell of the labeled molecule is identified. The location of the labeled molecule in the cell in the presence of the compound(s) is compared to the location of the labeled molecule in the cell in the absence of the compound(s). The difference is correlated between (1) the location of the labeled molecule in the cell in the presence of the compound and (2) the location of the labeled molecule in the cell in the absence of the compound(s) to modulation of GPCR internalization.

In the methods of the present invention, the GPCR may be complexed with one or more other molecules. The GPCR may be complexed with arrestin. In the methods of the present invention, the GPCR/arrestin complex may be labeled. The GPCR may be labeled or the arrestin may be labeled. In the methods of the present invention, the labeled molecule may be localized at the plasma membrane, endocytic vesicles, or endosomes.

The compounds identified by the present methods may alter PI3K catalytic activity. The compound may alter formation of the PI3K/βARK1 complex. The compound may inhibit GPCR internalization, for example by inhibiting βARK1-mediated translocation of PI3K to the plasma membrane of the cell. The location of PI3K within the cell may be directly identified.

In the methods of the present invention, the βARK1 may form a complex with PI3K. The βARK1/PI3K, the βARK1, or the PI3K may labeled. The PI3K may be localized in the cytosol or the plasma membrane.

The compounds identified by the present methods may inhibit formation of a βARK1/PI3K complex, wherein the βARK1/PI3K complex may be labeled. The βARK1 or the PI3K may be labeled.

In the methods of the present invention, the cell may comprise adaptin. The adaptin may be labeled.

The invention also relates to modified PI3Ks, wherein GPCR desensitization is altered when the modified PI3K is expressed in a cell. The modified PI3K may comprise or may lack a HEAT domain. The modified PI3K may lack catalytic activity.

In a further aspect of the present invention, the modified PI3K may comprise a polypeptide with the amino acid sequence of SEQ ID NO:2, 4, 6, 8, or 9. The modified PI3K may be a class IB PI3K or a class IA PI3K.

An additional aspect of the present invention is a modified PI3K, wherein the ability of a GPCR to bind adaptin may be altered when the modified PI3K is expressed in a cell. The modified PI3K may be PI3Kγ.

A further aspect of the present invention is a modified PI3K, wherein cellular Ptdlns (3,4,5) levels may be altered when the modified PI3K is expressed in a cell.

An additional aspect of the present invention is a modified PI3K, wherein the ability of wild-type PI3K to bind βARK1 may be altered. In a further aspect, the modified PI3K may have, or may lack, the ability to bind βARK1. The modified PI3K may be conjugated to a detectable molecule.

A further aspect of the present invention is a modified βARK1 which lacks the ability to bind PI3K.

The present invention also relates to an isolated nucleic acid sequence which encodes a modified PI3K. The nucleic acid molecule, in particular a recombinant DNA molecule or cloned gene, encoding the modified PI3K may have a nucleotide sequence or may be complementary to a DNA sequence shown in FIG. 2 (SEQ ID NO: 1, 3, 5, or 7). The present invention also includes expression vector comprising the nucleic acid operably linked to an expression control sequence.

The present invention also relates to host cells containing the expression vector. The host cell may comprise the nucleic acid integrated in its genome.

A further aspect of the present invention is a non-human transgenic animal which expresses a modified PI3K. The non-human transgenic animal may be a mouse or the non-human transgenic animal may be a primate, a feline, a canine, a porcine, a bovine, a caprine, or an ovine.

An additional aspect of the present invention are compounds identified by the above methods.

The present invention includes, an assay system which may be prepared in the form of a test kit for the quantitative analysis of the extent of the present of the βARK1/PI3K complex in a biological sample. The system or test kit may comprise an antibody which recognizes and binds to the βARK1/PI3K complex and reagents which detect the antibody that binds to the βARK1/PI3K complex.

An additional aspect of the present invention is an isolated immunoglobulin which recognizes and binds to a βARK1/PI3K complex. The immunoglobulin may be a monoclonal antibody, a chimeric antibody, a human antibody, a bispecific antibody, a humanized antibody, a primatized antibody, or an antibody fragment. The antibody fragment may be Fab, Fab′, F(ab′)2, F(v), or scFv.

A further aspect of the present invention is a method of altering GPCR internalization which comprises providing an effective amount of LY294002. The method of altering GPCR internalization, may comprise providing an effective amount of wortmannin.

An additional aspect of the present invention is a method of preventing and/or treating a disease associated with GPCR activity in mammals, including providing to a mammal a therapeutically effective amount of a compound identified in the above methods and a pharmaceutically acceptable carrier. A further aspect is a method of preventing and/or treating a disease associated with PI3K activity in mammals, including providing to a mammal a therapeutically effective amount of a compound identified in the above methods and a pharmaceutically acceptable carrier. A further aspect is a method of preventing and/or treating a disease associated with GPCR activity in mammals, including providing to a mammal administering to a mammal an amount of the isolated nucleic acid of the present invention sufficient to reduce or alleviate symptoms of said disease. The treated disease may be a cardiovascular disease, heart failure, asthma, nephrogenic diabetes insipidus, or hypertension.

### BRIEF DESCRIPTION OF DRAWINGS

The objects and advantages of the invention will be understood by reading the following detailed description in conjunction with the drawings in which:

FIG. 1 is list of GPCRs.

FIG. 2 is a listing of particular PI3K and HEAT nucleic acid and amino acid sequences. FIG. 2A is the nucleic acid sequence of the FLAG epitope-tagged HEAT domain of PI3Kγ, SEQ ID NO: 1. FIG. 2B is the amino acid sequence of the FLAG epitope-tagged HEAT domain of PI3Kγ, SEQ ID NO: 2. FIG. 2C the nucleic acid sequence of HA epitope-tagged PI3Kγ, SEQ ID NO: 3. FIG. 2D is the amino acid sequence of the HA epitope-tagged PI3Kγ, SEQ ID NO: 4. FIG. 2E is the nucleic acid sequence of the HA epitope-tagged PI3Kγ ATP binding site deletion, SEQ ID NO: 5. FIG. 2F is the amino acid sequence of the HA epitope-tagged PI3Kγ ATP binding site deletion, SEQ ID NO: 6. FIG. 2G is the nucleic acid sequence of PI3Kα, SEQ ID NO: 7. FIG. 2H is the amino acid sequence of PI3Kα, SEQ ID NO: 8. FIG. 2I is the amino acid sequence of the HEAT domain of PI3Kγ, SEQ ID NO: 9.

FIG. 3 illustrates that β3ARK1 and PI3K interact to form a cytosolic complex. FIG. 3A is an autoradiograph showing PI3K activity associated with βARK1 following βARK1 immunoprecipitation from a cytosolic extract. FIG. 3B is a bar graph representing the βARK1-associated PI3K activity, quantified by phosphorimaging the TLC plates of four independent experiments. FIG. 3C shows immunoprecipitations (IP) from cytosolic extracts with an anti-HA or anti-βARK1 monoclonal antibody and immunoblotted (IB) with a βARK1 monoclonal (upper panel) or anti-HA monoclonal antibody (lower panel). FIG. 3D is an autoradiograph of PI3K activity associated with immunoprecipitated βARK1 from a cytosolic extract. FIG. 3E is an autoradiograph showing PI3K activity associated with immunoprecipitated βARK1 from cytosolic extracts from cells with and without treatment with 100 nM wortmannin (Wort) for 15 minutes prior to lysis.

FIG. 4 illustrates that βARK1 and PI3Kγ form a complex in the cytosol and translocate to the membrane on agonist stimulation in a Gβγ dependent manner. FIG. 4A is an autoradiograph illustrating PI3K activity associated with βARK1 immunoprecipitated from the membrane and cytosol following isoproterenol stimulation (ISO, 10 μM) for a period of 2 min. In FIG. 4B, βARK1 was immunoprecipitated from the membrane and cytosolic fractions with a βARK1 monoclonal antibody and immunoblotted with an anti-HA monoclonal antibody. FIG. 4C is an autoradiograph showing PI3K activity associated with βARK1 immunoprecipitated, following isoproterenol stimulation, from membrane and cytosol with or without βARKct. FIG. 4D is a bar graph showing βARK1 associated PI3K activity quantified by phosphorimaging the TLC plates from four independent experiments. Results are expressed as fold over basal (no isoproterenol treatment). *p<0.05 vs. membrane fraction without ISO treatment. FIG. 4E shows βARK1 and βARKct associated with PI3K immunoprecipitated from cytosolic extracts.

FIG. 5 illustrates that βARK1 translocates PI3Kγ to the β-adrenergic receptor. FIG. 5A is an autoradiograph of PI3K activity associated with immunoprecipitated SEAR following treatment with 10 μM isoproterenol, which in FIG. 5B is quantified by phosphorimaging. FIG. 5B is an autoradiograph of PI3K activity associated with immunoprecipitated β₂AR following treatment with 10 μM isoproterenol, which in FIG. 5D is quantified by phosphorimaging. FIG. 5E shows PI3K activity associated with βAR in presence and absence of wortmannin.

FIG. 6 illustrates that PI3K activity is required for βAR sequestration. FIG. 6A shows agonist-promoted PI3K activity and receptor sequestration in the presence and absence of wortmannin. FIG. 6B shows agonist-promoted PI3K activity and receptor sequestration in the presence and absence of wortmannin in cells cotransfected with β₂AR, along with empty vector (▪), PI3Kγ (□), or ΔPI3Kγ (◯) cDNAs. FIG. 6C shows β-arrestin2-GFP recruitment to the membrane on isoproterenol stimulation (1 μM). Marked redistribution of β-arrestin2-GFP to the membrane occurs within 2.5 min. Arrows on the 5 min panel highlights regions of translocated β-arrestin2-GFP. The upper panel is an immunoblot for HA from extracts of the same cells showing equal levels of HA-PI3Kγ and HA-ΔPI3Kγ expression. FIG. 6D agonist (ISO, 5 min) promoted β₂AR phosphorylation in transfected cells after ³²Pi metabolic labeling for 1 hr prior to stimulation (upper panel). Lower panel, immunoblot for HA showing equal levels of expression of HA-PI3Kγ and HA-ΔPI3Kγ.

FIG. 7 illustrates that βARK1 and PI3K form a complex in heart. FIG. 7A is a PI3K immunoblot of βARK1 immunoprecipitate of myocardial extract. FIG. 7B is an autoradiograph showing PI3K activity associated with βARK1 immunoprecipitate of myocardial extract, in presence and absence of agonist. FIG. 7C is an autoradiograph showing βARK1 associated PI3K activity of myocardial extracts from the sham (S) and transverse aortic constricted (T) hearts, in the absence or presence of wortmannin. FIG. 7D shows PI3Kγ associated with immunoprecipitated βARK1 from myocardial extracts of sham (S) and T hearts.

FIG. 8 illustrates that βARK1 directly interacts with the HEAT domain of PI3K. FIG. 8A is a schematic representation of full length PI3 Kp110γ and mutants. FIG. 8B shows βARK1 co-immunoprecipitated with FLAG-HEAT and PI3ΔHEAT. As shown in FIG. 8C, beads with bound GST alone or GST-HEAT fusion proteins were incubated with purified βARK1, bound material was run on SDS-PAGE and immunoblotted with βARK1 monoclonal antibody. In FIG. 8D, beads with bound GST-HEAT fusion protein or βARK1 protein were incubated with purified Gβγ subunits of G-protein, and bound material was separated by SDS-PAGE electrophoresis followed by Immunodetection of Gβ.

FIG. 9 illustrates that over-expression of the 197 amino acid HEAT domain of PI3K competes for endogenous PI3K binding to βARK1. FIG. 9A is an autoradiograph showing PI3K activity associated with immunoprecipitated βARK1 in the presence of increasing amounts of HEAT cDNA. FIG. 9B is the summary results of n=5 experiments. *p<0.0005. The data was normalized to βARK1 associated PI3K activity in cells transfected with βARK1 only. FIG. 9C is an autoradiograph of PI3K activity associated with immunoprecipitated βARK1 from cells transfected with βARK1 or βARK1 and HEAT or βARK1 and PI3KΔHEAT encoding cDNAs. FIG. 9D are the summary results of n=3 experiments. *p<0.001. The data was normalized to βARK1 associated PI3K activity in cells transfected with βARK1 only.

FIG. 10 illustrates that over-expression of HEAT blocks βARK1 mediated translocation of PI3K to the membrane and to the β₂AR. FIG. 10A is an autoradiograph of PI3K activity associated with immunoprecipitated βARK1 from the membrane fraction of unstimulated and stimulated (10 μM isoproterenol for 2 min) cells transfected with βARK1 or βARK1 and HEAT encoding cDNAs. FIG. 10B is a βARK1 immunoblot of βARK1 immunoprecipitations of the membrane fraction of cells transfected with βARK1 or βARK1 and HEAT encoding cDNAs, in presence or absence of agonist. FIG. 10C is an autoradiograph of PI3K activity associated with immunoprecipitated FLAG-β₂AR from cells transfected with β₂AR or β₂AR and HEAT encoding cDNAs, upon agonist stimulation.

FIG. 11 illustrates the attenuation of β₂AR sequestration upon co-expression of HEAT. FIG. 11A shows agonist-promoted (1 μM isoproterenol) β₂AR sequestration by ¹²⁵I-cyanopindolol binding carried out in HEK 293 cells transfected with β₂AR and either the empty vector (▪) or HEAT (□) encoding cDNAs over a time course of 0-30 min. FIG. 11B shows the endocytosis of β₂AR-YFP in live cells monitored for 10 min following isoproterenol (10 μM) stimulation in the absence or presence of HEAT protein co-expression using confocal microscopy. Panels on the left represent cells transfected with the β₂AR-YFP alone (panels 1, 2 and 3 show the same cell monitored at 0, 5 and 10 min following stimulation. Panel 7 is an example of another cell after 10 min of isoproterenol). Panels on the right represent cell transfected with β₂AR-YFP and HEAT (panels 4,5 and 6 show the same cell monitored as above. Panel 8 is an example of another cell after 10 min of isoproterenol). FIG. 11C shows dual staining in cells transfected with plasmids containing HA-β₂AR and GFP-HEAT cDNAs. After 10 min of 10 μM isoproterenol, cells were fixed with 4% paraformaldehyde, stained for HA-β₂AR receptor with Texas Red and HEAT was visualized by GFP fluorescence. Panel 1 shows two cells, one with only membrane distribution of HA-β₂AR, and another cell with complete redistribution of β₂AR s into aggregates (arrowheads). Panel 2, shows that the cell with GFP fluorescence (i.e. HEAT protein expression) failed to undergo β₂AR internalization, whereas the cell not expressing HEAT-GFP showed HA-β₂AR aggregates. Panel 3 is an overlay of panels 1 and 2.

FIG. 12 illustrates that the over-expression of HEAT neither inhibits β-arrestin2 recruitment to the receptor complex nor downstream PI3K signaling. In FIG. 12A, confocal microscopy was used to visualize fluorescence in HEK 293 double stably expressing (β₂AR-HA and β-arrestin2-GFP) cells transfected with the FLAG-HEAT plasmid. Following stimulation with 10 μM isoproterenol for 10 min, cells were fixed and stained with Texas Red. All cells show β-arrestin2-GFP (βarr2-GFP) fluorescence whereas a smaller percentage show Texas Red staining of the FLAG epitope. Panels 1 and 2 show that in the absence of isoproterenol, both HEAT and β-arrestin2-GFP were distributed in the cytoplasm. Panels 3 and 4, following the addition of isoproterenol, the presence of HEAT protein had no effect on the marked re-distribution of β-arrestin-GFP fluorescence to the membrane (arrowheads). In FIG. 12B, clarified lysates of HEK 293 cells transfected with vector or HEAT encoding cDNAs treated with various agonists were immunoblotted with phospho-PKB. MOCK, treated with ascorbic acid, IGF, treated with insulin growth factor (10 nM), Car, treated with carbachol (1 mM), ISO, treated with isoproterenol dissolved in ascorbic acid (10 μM). Lower panel shows expression of HEAT protein in transfected cells.

FIG. 13 illustrates that the generation of D-3 phosphatidylinositols are necessary for the efficient recruitment of adaptin to the β₂AR complex. In FIG. 13A, cells transfected with FLAG-β₂AR, or FLAG-β₂AR and HEAT encoding cDNAs were treated with isoproterenol (10 μM) for 0, 2, 5, and 10 min, β₂AR was immunoprecipitated using the FLAG epitope from the cells lysates, and immunoblotted with antibodies for AP-2 adaptin protein and clathrin. Lower panels show expression of β₂AR and HEAT protein in transfected cells and equal quantities of adaptin and clathrin loading. IgG, represents heavy chain of the antibody. C, positive control for clathrin and AP2 adaptin proteins. FIG. 13B shows adaptin immunoblots of lysates of FLAG-β₂AR expressing stable cells treated with LY294002 (a selective PI3K inhibitor) for 15 min prior to isoproterenol stimulation. The cells were stimulated for 0, 2, 5 and 10 min and b2AR was immunoprecipitated using a FLAG epitope. FIG. 13C is the summary results of densitometric analysis of adaptin recruitment to β₂AR in presence and absence of LY294002 (n=7). The data is represented as fold over basal. In FIG. 13D, FLAG-β₂AR expressing stable cells were treated with the permeablizing reagent saponin and the phospholipids as shown, followed by isoproterenol for 5 min. FLAG epitope was immunoprecipitated from the cell lysates and blotted with adaptin antibodies. The upper panel shows cells treated with Ptdlns (4,5) P₂, while lower panel were treated with Ptdlns (3,4,5) P₃. IgG, heavy chain of antibody. FIG. 13E is the summary results of densitometric analysis of adaptin recruitment to β₂AR complex in presence of Ptdlns (4,5) P₂ or Ptdlns (3,4,5) P₃ (n=7). Data is represented as fold over basal.

FIG. 14 illustrates the attenuation of V2R sequestration upon co-expression of HEAT.

FIG. 15 illustrates the attenuation of AT1AR sequestration upon co-expression of HEAT.

FIG. 16 illustrates that PI3K specifically associates with βARK, and not with GRK5.

FIG. 17 shows the catalytic inactive PI3Kγ construct used to make catalytic inactive PI3Kγ transgenic mice.

FIG. 18 shows the effect of pressure overload on cardiac function for control versus transgenic mice.

FIG. 19 shows, graphically, the effect of pressure overload on cardiac function for control versus transgenic mice.

FIG. 20 is an autoradiograph illustrating the decreased PI3K activity associated with immunoprecipitated βARK1, upon chronic pressure overload, for control versus transgenic mice.

FIG. 21 is a graphic representation of the decreased % loss in isoproterenol responsiveness of transgenic versus control mice upon chronic treatment with isoproterenol for seven days.

FIG. 22 is an autoradiograph illustrating the decreased PI3K activity associated with βARK1 immunoprecipitated from membranes, upon chronic pressure overload, for control versus transgenic mice.

### DETAILED DESCRIPTION

In accordance with the present invention there may be employed conventional molecular biology, microbiology, immunology, and recombinant DNA techniques-within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (2000)]; “Immobilized Cells And Enzymes” [IRL Press, (1986)]; B. Perbal, “A Practical Guide To Molecular Cloning” (1984); Using Antibodies: A Laboratory Manual: Portable Protocol No. I, Harlow, Ed and Lane, David (Cold Spring Harbor Press, 1998); Using Antibodies: A Laboratory Manual, Harlow, Ed and Lane, David (Cold Spring Harbor Press, 1999).

Unless otherwise stated, the following terms used in the specification and claims have the meanings given below:

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by-mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgamo sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences having the same amino acid sequence as SEQ ID NO: 2, 4, 6, 8, or 9, but which are degenerate to SEQ ID NO: 2, 4, 6, 8, or 9. By “degenerate to” is meant that a different three-letter codon is used to specify a particular amino acid.

“Arrestin” means all types of naturally occurring and engineered variants of arrestin, including, but not limited to, visual arrestin (sometimes referred to as Arrestin 1), βarrestin 1 (sometimes referred to as Arrestin 2), and βarrestin 2 (sometimes referred to as Arrestin 3).

A “HEAT” domain is a domain important for protein-protein interactions in proteins such as the protein phosphatase 2A, importin-b and target of rapamycin (TOR). The HEAT domain motif is named after proteins containing these repeats: Huntingtin Elongation Factor 3, A subunit of protein phosphatase 2A and TOR. Certain HEAT domains are described in Neuwald and Hirano, Genome Research, 10:1445-1452 (2000), which is hereby incorporated by reference in its entirety. The present inventors demonstrated that the HEAT domain of PI3K is an important interacting domain with βARK1.

“βARK1” is a GRK termed β-adrenergic receptor kinase 1, also called GRK2.

“βAR” is a GPCR termed a β-adrenergic receptor.

“Phosphoinositide 3-Kinases” (PI3Ks) are a conserved family of lipid kinases that catalyze the addition of phosphate on the third position of the inositol ring. PI3Ks include class IA and IB, α, and γ PI3Ks.

“Internalization” of a GPCR is the intracellular translocation of a GPCR from the membrane.

“Carboxyl-terminal tail” means the carboxyl-terminal tail of a GPCR. The carboxyl-terminal tail of many GPCRs begins shortly after the conserved NPXXY motif that marks the end of the seventh transmembrane domain (i.e. what follows the NPXXY motif is the carboxyl-terminal tail of the GPCR). The carboxyl-terminal tail may be relatively long (approximately tens to hundreds of amino acids), relatively short (approximately tens of amino acids), or virtually non-existent (less than approximately ten amino acids). As used herein, “carboxyl-terminal tail” shall mean all three variants (whether relatively long, relatively short, or virtually non-existent).

“Class-A receptors” preferably do not translocate to endocytic vesicles or endosomes in HEK-293 cells.

“Class B receptors” preferably do translocate to endocytic vesicles or endosomes in HEK-293 cells.

“DACS” mean any desensitization active compounds. Desensitization active compounds are any compounds that influence the GPCR desensitization mechanism by either stimulating or inhibiting the process. DACs influence the GPCR desensitization pathway by acting on any cellular component of the process, as well as any cellular structure implicated in the process, including but not limited to, arrestins, GRKs, GPCRs, PI3K, AP-2 protein, clathrin, protein phosphatases, and the like. DACs may include, but are not limited to, compounds that inhibit arrestin translocating to a GPCR, compounds that inhibit arrestin binding to a GPCR, compounds that stimulate arrestin translocating to a GPCR, compounds that stimulate arrestin binding to a GPCR, compounds that inhibit GRK phosphorylation of a GPCR, compounds that stimulate GRK phosphorylation of a GPCR, compounds that inhibit protein phosphatase dephosphorylation of a GPCR, compounds that stimulate protein phosphatase dephosphorylation of a GPCR, compounds that regulate the release of arrestin from a GPCR, antagonists of a GPCR, inverse agonists and the like. DACs preferably inhibit or stimulate the GPCR desensitization process without binding to the same ligand binding site of the GPCR as traditional agonists and antagonists of the GPCR. DACs act independently of the GPCR, i.e., they do not have high specificity for one particular GPCR or one particular type of GPCRs.

“Detectable molecule” means any molecule capable of detection by spectroscopic, photochemical, biochemical, immunochemical, electrical, radioactive, and optical means, including but not limited to, fluorescence, phosphorescence, and bioluminescence and radioactive decay. Detectable molecules include, but are not limited to, GFP, luciferase, β-galactosidase, rhodamine-conjugated antibody, and the like. Detectable molecules include radioisotopes, epitope tags, affinity labels, enzymes, fluorescent groups, chemiluminescent groups, and the like. Detectable molecules include molecules which are directly or indirectly detected as a function of their interaction with other molecule(s).

“GFP” means Green Fluorescent Protein which refers to various naturally occurring forms of GFP which may be isolated from natural sources or genetically engineered, as well as artificially modified GFPs. GFPs are well known in the art. See, for example, U.S. Pat. Nos. 5,625,048; 5,777,079; and 6,066,476. It is well understood in the art that GFP is readily interchangeable with other fluorescent proteins, isolated from natural sources or genetically engineered, including but not limited to, yellow fluorescent proteins (YFP), red fluorescent proteins (RFP), cyan fluorescent proteins (CFP), blue fluorescent proteins, luciferin, UV excitable fluorescent proteins, or any wave-length in between. As used herein, “GFP” shall mean all fluorescent proteins known in the art.

“Unknown or Orphan Receptor” means a GPCR whose function and/or ligands are unknown.

“NPXXY motif” means a conserved amino acid motif that marks the end of the seventh transmembrane domain. The conserved amino acid motif begins with asparagine and proline followed by two unspecified amino acids and then a tyrosine. The two unspecified amino acids may vary among GPCRs but the overall NPXXY motif is conserved.

“Downstream” means toward a carboxyl-terminus of an amino acid sequence, with respect to the amino-terminus.

“Upstream” means toward an amino-terminus of an amino acid sequence, with respect to the carboxyl-terminus.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “immunoglobulin” includes antibodies and antibody fragments with immunogenic activity. Preferred immunogenic activity is where the immunoglobulin binds to a modified GPCR. An even more preferable immunoglobulin is one that can distinguish between a modified GPCR and a wild-type GPCR. And the most preferable immunoglobulin is that which binds to a modified DRY motif or a DRY motif of a GPCR. The term “antibody” refers to immunoglobulins, including whole antibodies as well as fragments thereof that recognize or bind to specific epitopes. The term antibody encompasses polyclonal, monoclonal, and chimeric antibodies, the last mentioned described in further detail in U.S. Pat. Nos. 4,816,397 and 4,816,567. The term “epitope” is used to identify one or more portions of an antigen or an immunogen which is recognized or recognizable by antibodies or other immune system components.

Exemplary immunoglobulins are intact immunoglobulin molecules, substantially intact immunoglobulin molecules and those portions of an immunoglobulin molecule that contains the paratope. Antibody fragments include those portions known in the art as Fab, Fab′, F(ab′)₂, F(v), and scFv which portions are preferred for use in the therapeutic methods described herein.

Fab and F(ab′)₂ portions of antibody fragments are prepared by the proteolytic reaction of papain and pepsin, respectively, on substantially intact antibodies by methods that are well-known. See for example, U.S. Pat. No. 4,342,566 to Theofilopolous et al. Fab′ antibody portions are also well-known and are produced from F(ab′)₂ portions followed by reduction of the disulfide bonds linking the two heavy chain portions as with mercaptoethanol, and followed by alkylation with a reagent such as iodoacetamide. An antibody containing intact antibody portions is preferred herein.

An “antibody combining site” is that structural portion of an antibody comprised of heavy and light chain variable and hypervariable regions that specifically binds antigen.

The phrase “monoclonal antibody” in its various grammatical forms refers to an antibody having only one species of antibody combining site capable of immunoreacting with a particular epitope on an antigen. A monoclonal antibody may therefore contain a plurality of antibody combining sites, each immunospecific for a different antigen, e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce some feature of pathology such as for example, elevated blood pressure, respiratory output, etc.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

“Hybridization” means hydrogen bonding, which may be Watson-Crick, Hoogsteen or reversed Hoogsteen hydrogen bonding, between complementary nucleoside or nucleotide bases. For example, adenine (A) and thymine (T) are complementary nucleobases which pair through the formation of hydrogen bonds.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tm with washes of higher stringency, if desired.

By “animal” is meant any member of the animal kingdom including vertebrates (e.g., frogs, salamanders, chickens, or horses) and invertebrates (e.g., worms, etc.). “Animal” is also meant to include “mammals.” Preferred mammals include livestock animals (e.g., ungulates, such as cattle, buffalo, horses, sheep, pigs and goats), as well as rodents (e.g., mice, hamsters, rats and guinea pigs), canines, felines, primates, lupine, camelid, cervidae, rodent, avian and ichthyes.

“Antagonist(s)” include all agents that interfere with wild-type and/or modified GPCR binding to an agonist, wild-type and/or modified GPCR desensitization, wild-type and/or modified GPCR binding arrestin, wild-type and/or modified GPCR endosomal localization, internalization, and the like, including agents that affect the wild-type and/or modified GPCRs as well as agents that affect other proteins involved in wild-type and/or modified GPCR signaling, desensitization, endosomal localization, resensitization, and the like.

“GPCR” means G protein-coupled receptor and includes GPCRs naturally occurring in nature, as well as GPCRs which have been modified. Such modified GPCRs are described in U.S. Ser. No. 09/993,844 and U.S. Ser. No. 10/054,616.

“Abnormal GPCR desensitization” and “abnormal desensitization” mean that the GPCR desensitization pathway is disrupted such that the balance between active receptor and desensitized receptor is altered with respect to wild-type conditions. Either there is more active receptor than normal or there is more desensitized receptor than wild-type conditions. Abnormal GPCR desensitization may be the result of a GPCR that is constitutively active or constitutively desensitized, leading to an increase above normal in the signaling of that receptor or a decrease below normal in the signaling of that receptor.

“Biological sample” is intended to include tissues, cells and biological fluids isolated from a subject, as well as tissues, cells and fluids present within a subject; wherein said sample can be blood, serum, a urine sample, a fecal sample, a tumor sample, a cellular wash, an oral sample, sputum, biological fluid, a tissue extract, freshly harvested cells, or cells which have been incubated in tissue culture.

“Concurrent administration,” “administration in combination,” “simultaneous administration,” or “administered simultaneously” mean that the compounds are administered at the same point in time or sufficiently close in time that the results observed are essentially the same as if the two or more compounds were administered at the same point in time.

“Conserved abnormality” means an abnormality in the GPCR pathway, including but not limited to, abnormalities in GPCRs, GRKs, arresting, AP-2 protein, clathrin, protein phosphatase and the like, that may cause abnormal GPCR signaling. This abnormal GPCR signaling may contribute to a GPCR-related disease.

“Desensitized GPCR” means a GPCR that presently does not have ability to respond to agonist and activate conventional G protein signaling. Desensitized GPCRs of the present invention do not properly respond to agonist, are phosphorylated, bind arrestin, constitutively localize in clathrin-coated pits, and/or constitutively localize to endocytic vesicles or endosomes.

“Desensitization pathway” means any cellular component of the desensitization process, as well as any cellular structure implicated in the desensitization process and subsequent processes, including but not limited to, arresting, GRKs, GPCRs, AP-2 protein, clathrin, protein phosphatases, and the like. In the methods of assaying of the present invention, the polypeptides may be detected, for example, in the cytoplasm, at a cell membrane, in clathrin-coated pits, in endocytic vesicles, endosomes, any stages in between, and the like.

“GPCR signaling” means GPCR induced activation of G proteins. This may result in, for example, cAMP production.

“G protein-coupled receptor kinase” (GRK) includes any kinase that has the ability to phosphorylate a GPCR.

“Homo sapien GPCR” means a naturally occurring GPCR in a Homo sapien.

“Inverse agonist” means a compound which, upon binding to the GPCR, inhibits the basal intrinsic activity of the GPCR. An inverse agonist is a type of antagonist.

An “isolated” or “purified” nucleic acid molecule or protein, biologically active portion thereof, or antibody is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e.; sequences located at the 5 and 3 ends of the nucleic acid) in the genomic DNA of the organism from which the nucleic acid is derived. For purposes of the invention, “isolated” when used to refer to nucleic acid molecules, excludes isolated chromosomes. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, or 5% (by dry weight) of another protein. When the protein or biologically active portion thereof is recombinantly produced, preferably, culture medium represents less than about 30%, 20%, 10%, or 5% of the volume of the protein preparation. When protein is produced by chemical synthesis, preferably the protein preparations have less than about 30%, 20%, 10%, or 5% (by dry weight) of chemical precursors or non-protein chemicals.

“Modified GRK” means a GRK modified such that it alters desensitization.

“Naturally occurring GPCR” means a GPCR that is present in nature.

“Odorant ligand” means a ligand compound that, upon binding to a receptor, leads to the perception of an odor including a synthetic compound and/or recombinantly produced compound including agonist and antagonist molecules.

“Odorant receptor” means a receptor protein normally found on the surface of olfactory neurons which, when activated (normally by binding an odorant ligand) leads to the perception of an odor.

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Primatized antibody” means a recombinant antibody containing primate variable sequences or antigen binding portions, and human constant domain sequences.

“Sensitized GPCR” means a GPCR that presently has ability to respond to agonist and activate conventional G protein signaling.

The present invention is related to methods of screening compounds for inhibitors of GPCR internalization. The present invention also provides methods for screening compounds for inhibitors of these protein functions and the compounds identified through screening, as well as the use of such inhibitors to treat disease.

The exposure of a GPCR to agonist produces rapid attenuation of its signaling ability that involves uncoupling of the receptor from its cognate heterotrimeric G-protein. The cellular mechanism mediating agonist-specific or homologous desensitization is a two-step process in which agonist-occupied receptors are phosphorylated by a G protein-coupled receptor kinases (GRKs) and then bind an arrestin protein.

It has been discovered that after agonists bind GPCRs, G-protein coupled receptor kinases (GRKs) phosphorylate intracellular domains of GPCRs. After phosphorylation, an arrestin protein associates with the GRK-phosphorylated receptor and uncouples the receptor from its cognate G protein. The interaction of the arrestin with the phosphorylated GPCR terminates GPCR signaling and produces a non-signaling, desensitized receptor.

The arrestin bound to the desensitized GPCR targets the GPCR to clathrin-coated pits for endocytosis (i.e., internalization) by functioning as an adaptor protein, which links the GPCR to components of the endocytic machinery, such as adaptor protein-2 (AP-2) and clathrin. The internalized GPCRs are dephosphorylated and are recycled back to the cell surface desensitized. The stability of the interaction of arrestin with the GPCR is one factor which dictates the rate of GPCR dephosphorylation, recycling, and resensitization. The involvement of GPCR phosphorylation and dephosphorylation in the desensitization process has been exemplified in U.S. Ser. No. 09/933,844, filed Nov. 5, 2001, the disclosure of which is hereby incorporated by reference in its entirety.

The present inventors have determined that a conserved abnormality(ies) in any part of the GPCR signaling pathway (e.g., internalization) may cause abnormal GPCR signaling leading to a GPCR-related disease. For example, components of the GPCR signaling pathway, including but not limited to, GPCRs, GRKs, arresting, protein phosphatases, AP-2 proteins, clathrin, and the like, may express a mutation(s). The mutation(s) may result in, for example, abnormal ligand binding, G protein coupling, GPCR trafficking, and the like, that may cause altered GPCR signaling.

Certain protein functions are required for GPCR internalization. The present inventors have determined that certain GRKs interact with Phosphoinositide 3-Kinases (PI3Ks), and that inhibition of this interaction alters GPCR internalization.

PI3Ks are a conserved family of lipid kinases that catalyze the addition of phosphate on the third position of the inositol ring. Stimulation of a variety of receptor tyrosine kinases and GPCRs results in the activation of PI3K and leads to an increase in the level of D-3 phosphatidylinositol (Ptdlns) phospholipids, which in turn are potent signaling molecules that modulate a number of diverse cellular effects including: cell proliferation, cell survival, cytoskeletal rearrangements and receptor endocytosis. In this context, GPCR stimulation leads to the activation of the IB subclass of PI3Ks mediated by the Gβγ subunits of G-proteins. Studies have suggested a role of phosphoinositides in the process of receptor internalization. For example, deletion of the phosphoinositide binding site from β-arrestin impairs GPCR endocytosis, and the binding of Ptdlns (3,4,5) P₃ and Ptdlns (4,5) P2 to AP-2 promotes targeting of the receptor-arrestin complex to clathrin-coated pits. Although the product of PI3K was previously known to play a role in endocytosis, the role of the PI3K protein itself for endocytosis was not previously known.

The present inventors have discovered that PI3K directly interacts with GRK2, and that this interaction promotes GPCR internalization. Moreover, the present inventors have identified the HEAT domain as the domain of PI3K that directly binds GRK2, and that inhibition of this interaction prevents GPCR internalization. Specifically, the interaction of PI3K and GRK2 regulates the translocation of PI3K to the agonist-occupied βAR. Because PI3K (and/or its lipid products) modulate the internalization of βARs, inhibition of PI3Kγ activity prevents GPCR internalization.

In accordance with the present invention, methods of screening compounds for inhibitors of GPCR internalization are provided. These compounds include those which target steps of GPCR internalization mediated by PI3K and its products. Identified compounds can be used to treat diseases associated with GPCR internalization.

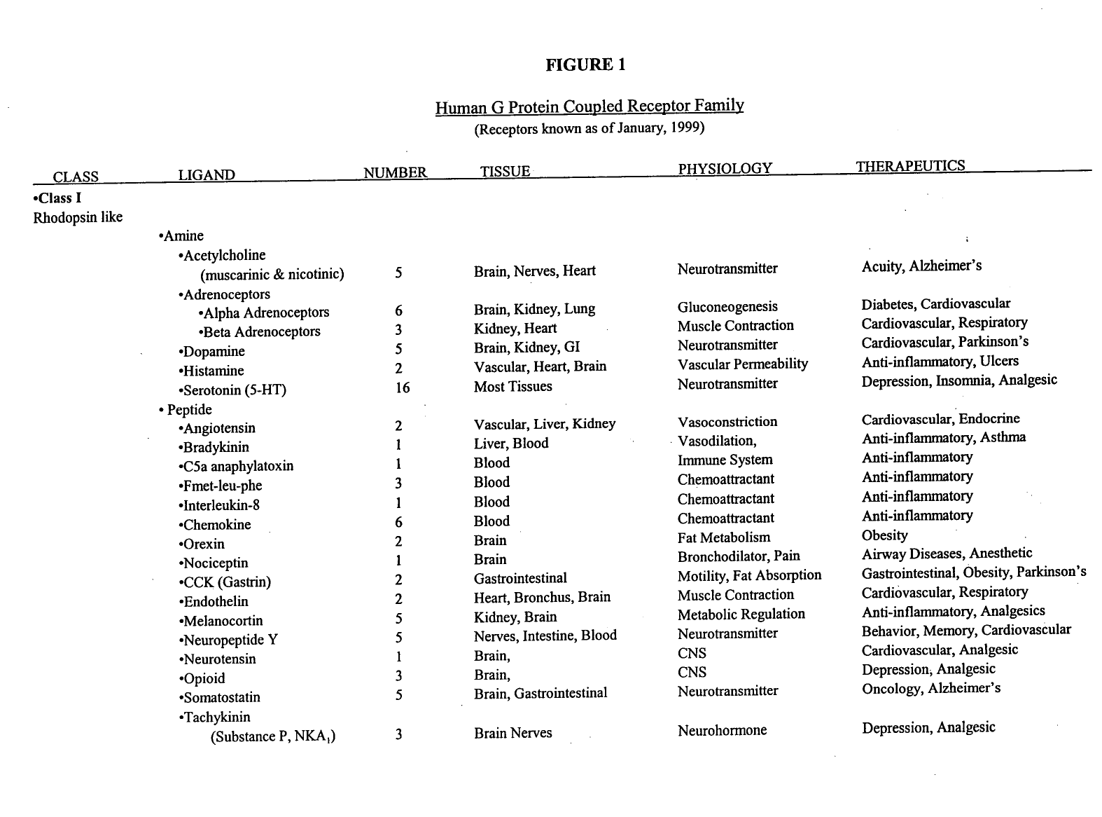

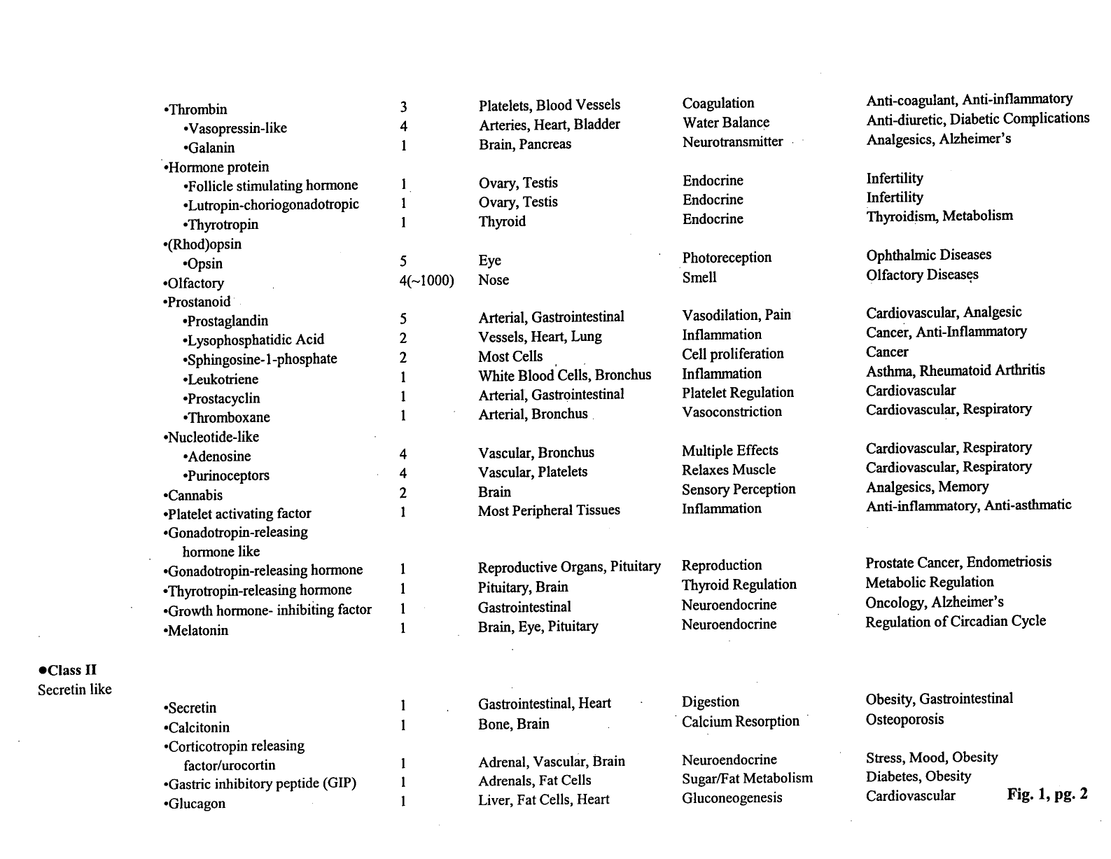

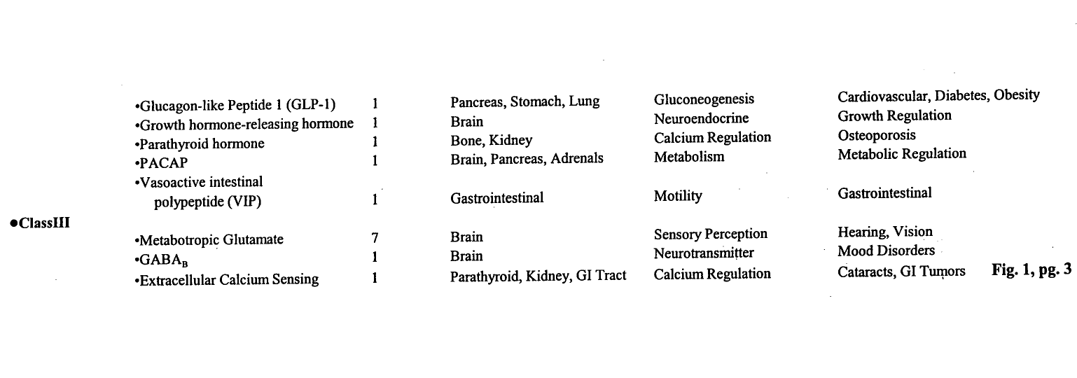

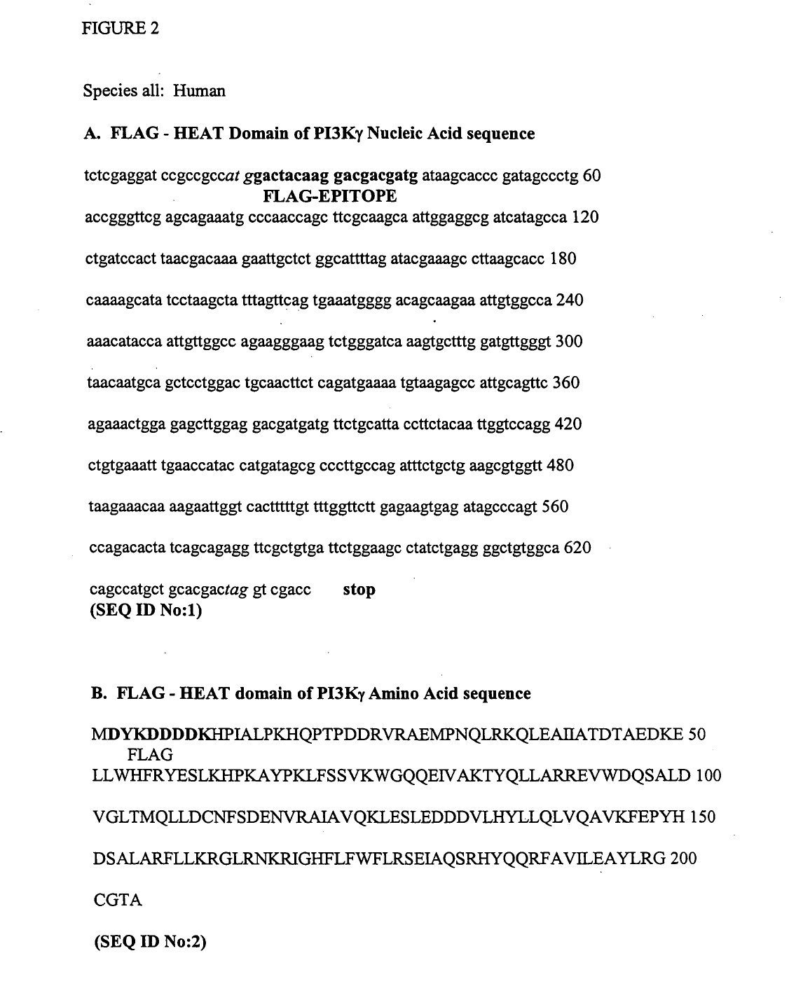

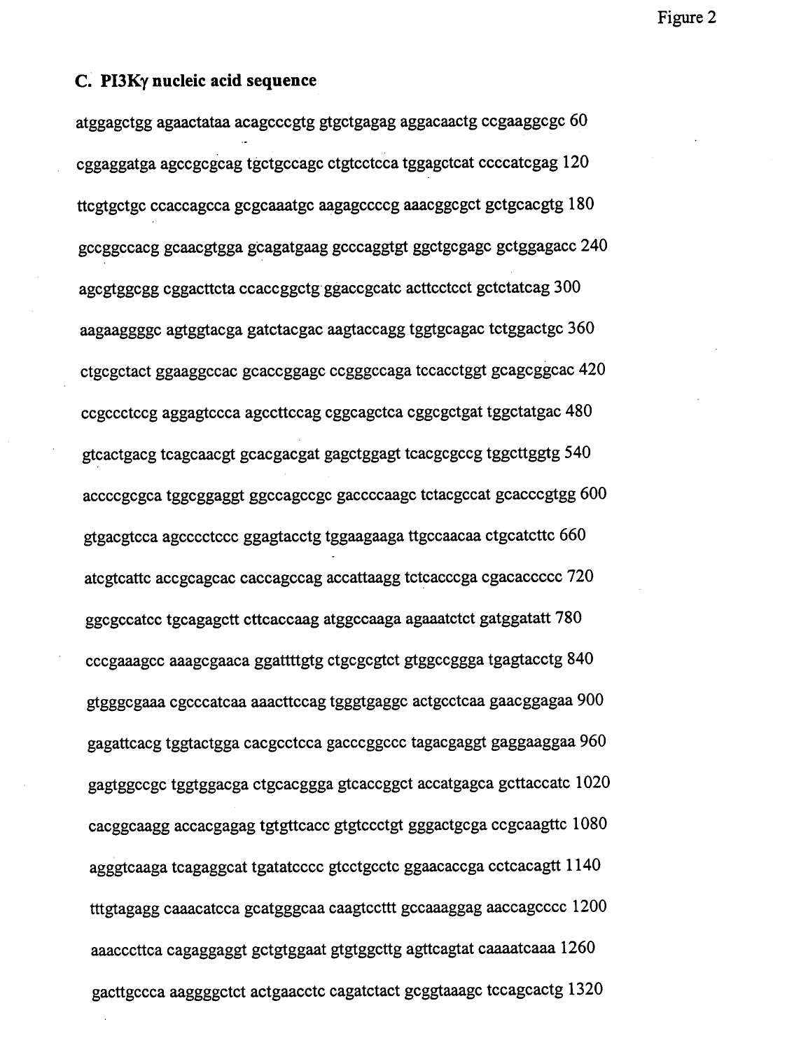

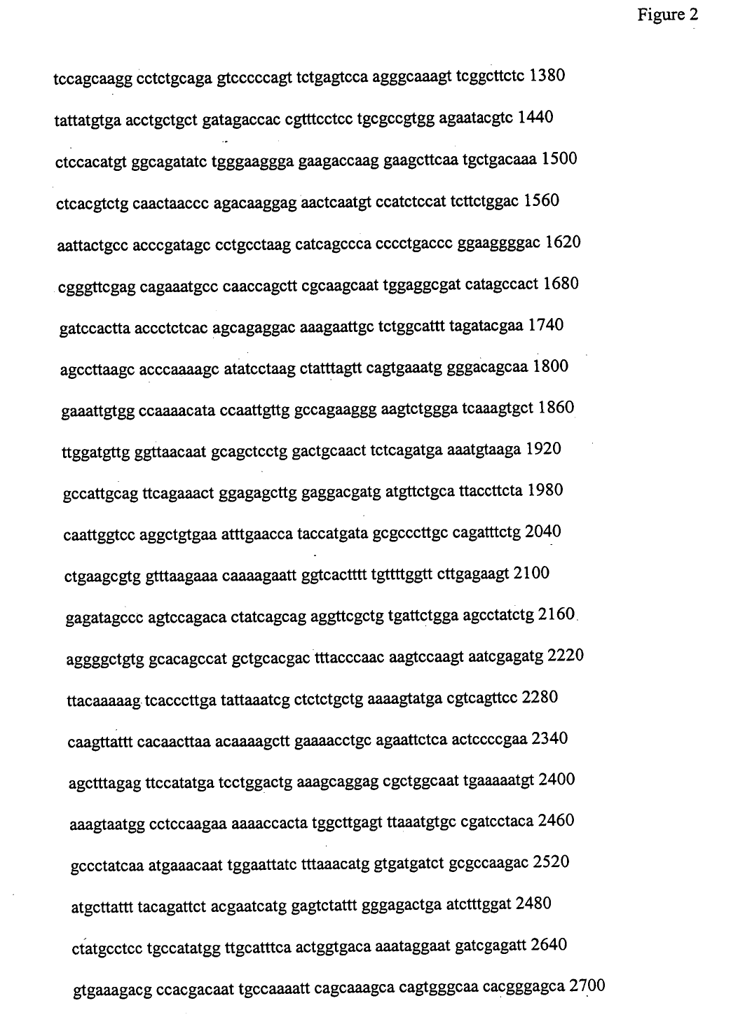

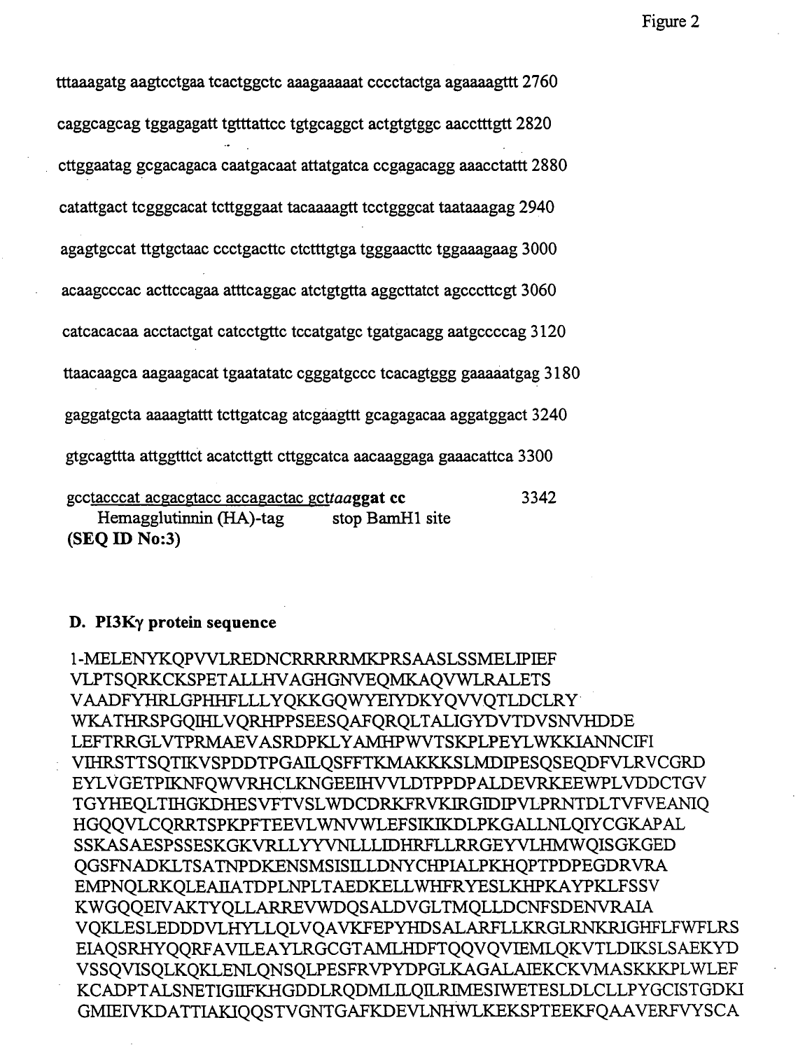

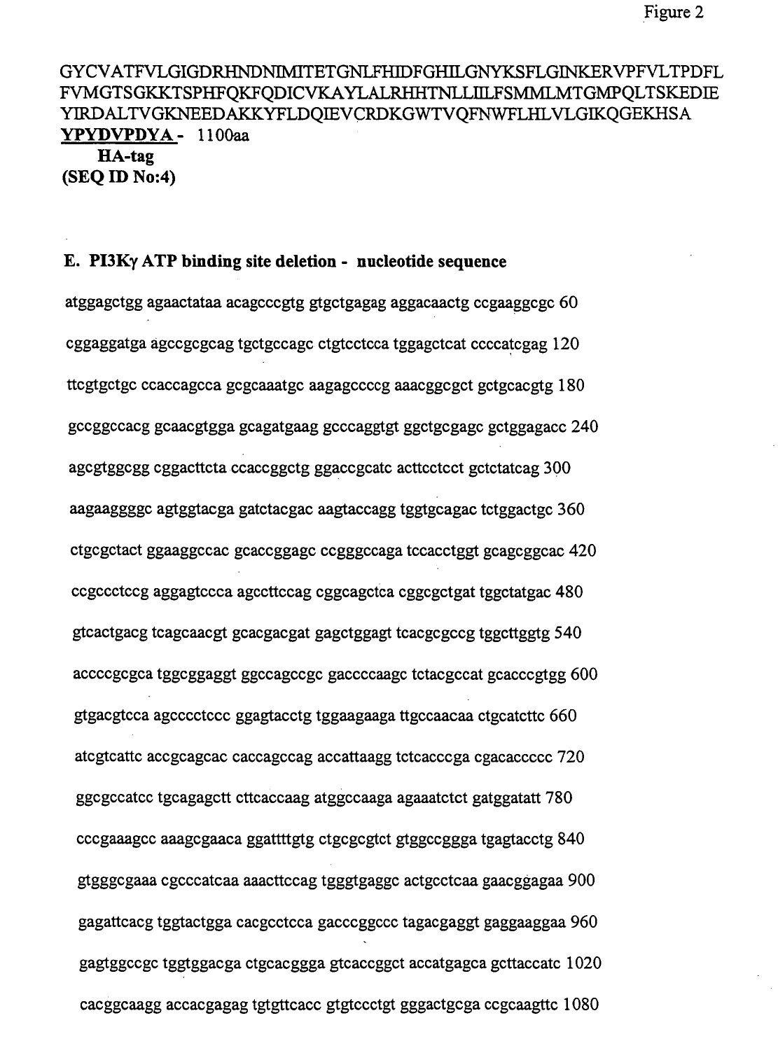

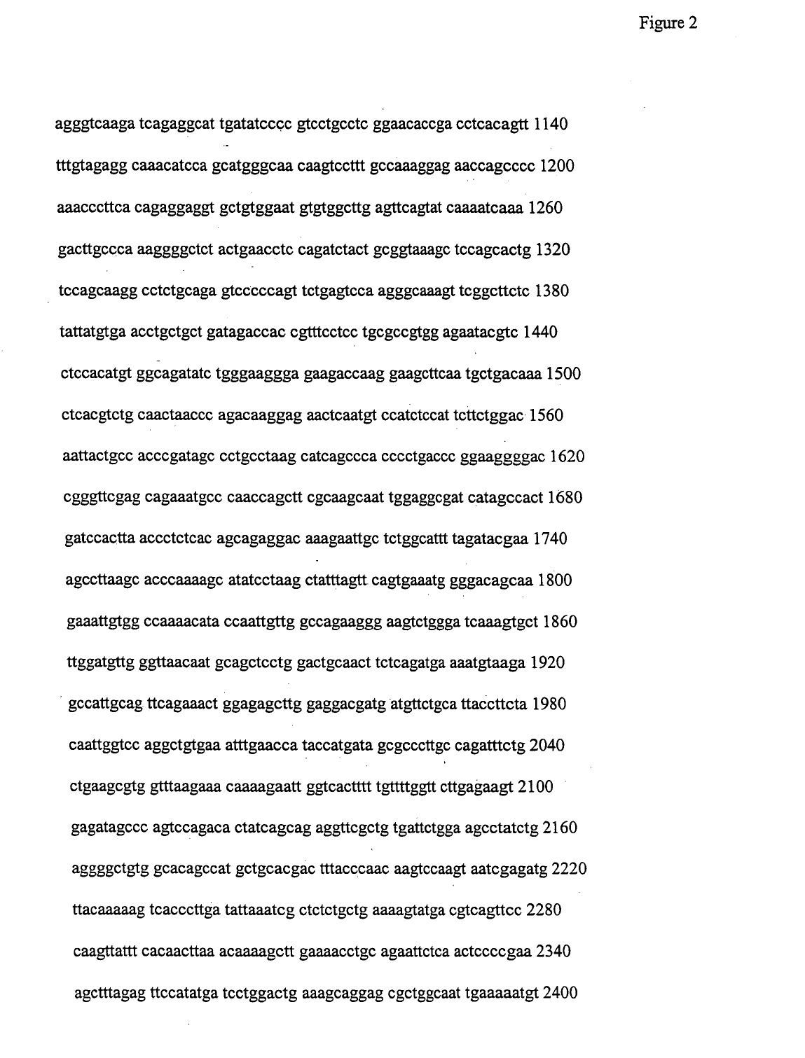

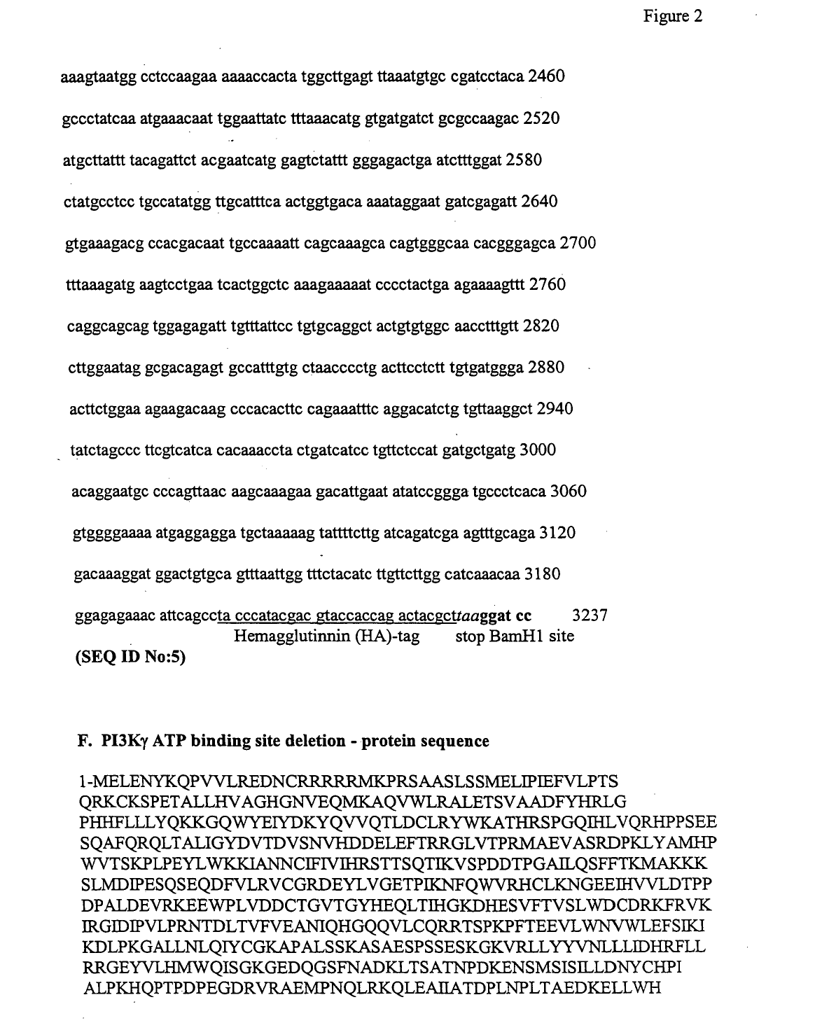

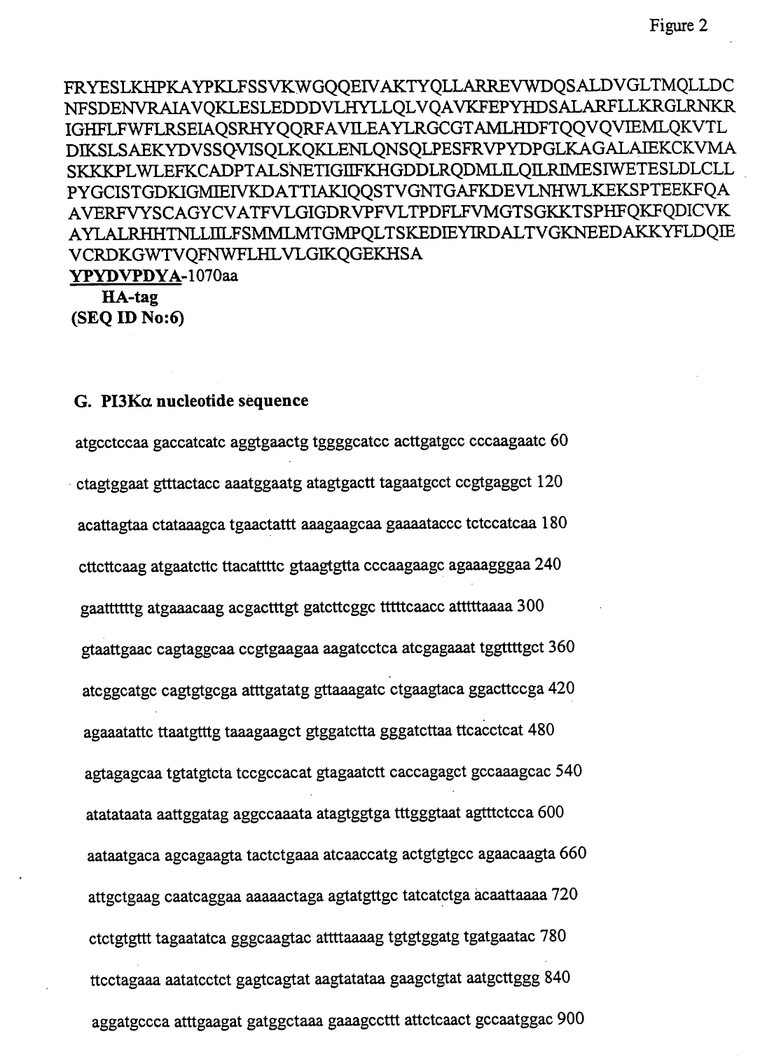

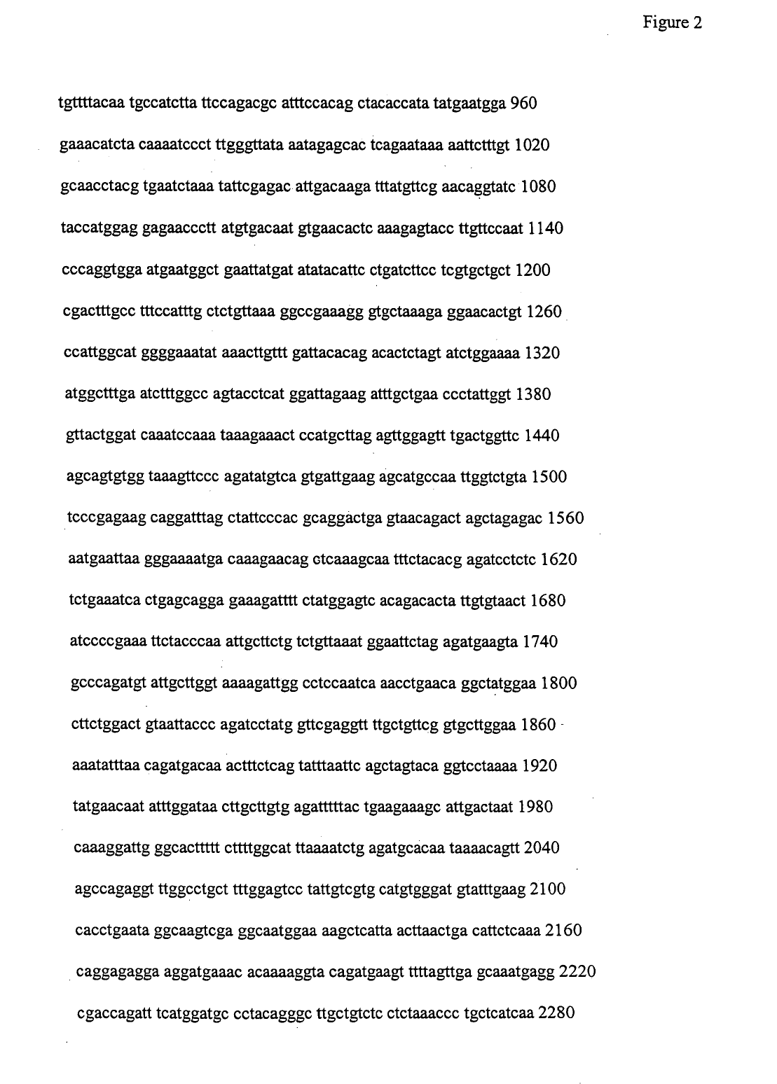

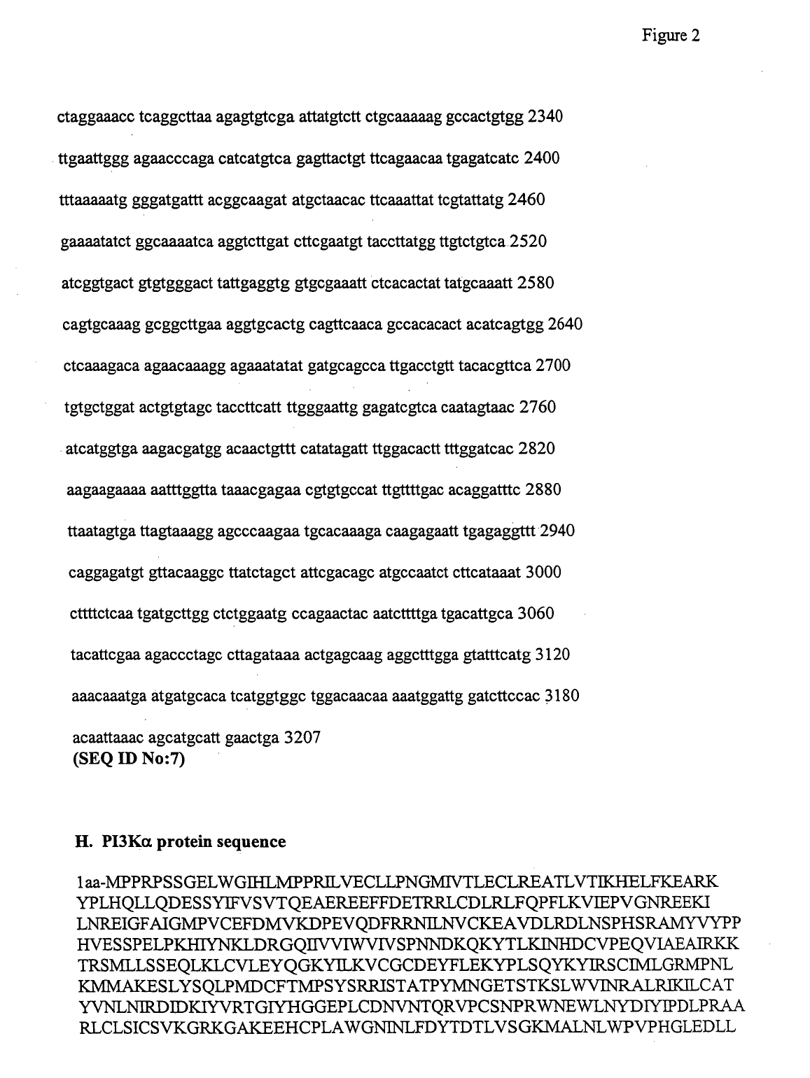

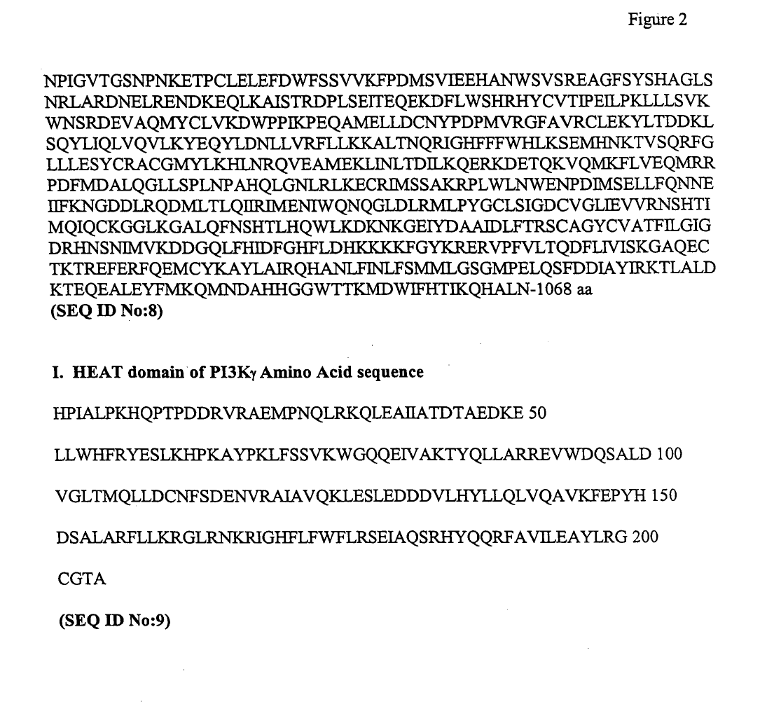

An illustrative, non-limiting list of known GPCRs with which the present invention may be used is contained in FIG. 1. The receptors are grouped according to classical divisions based on structural similarities and ligands.

By way of example, the major classes of GPCRs for known receptors are Class A receptors which preferably do not target to endocytic vesicles or endosomes in HEK-293 cells, and Class B receptors which preferably do target to endocytic vesicles or endosomes in HEK-293 cells.

GPCRs have been implicated in a number of disease states, including, but not limited to cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis, chronic inflammatory bowel disease, glaucoma, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (for example, cardiac malformations), neurodegenerative diseases such as Parkinson's Disease and Alzheimer's Disease, multiple sclerosis, Epstein-Barr infection and cancer. As such, modulation of GPCR internalization is a mechanism for ameliorating these disease states.

Most preferably, the methods of the present invention may be used to treat diseases modulated by the PI3K/βARK1 complex. For example, cardiac disease, Nephrogenic Diabetes Insipidus, and hypertension may be modulated by compounds identified by methods of the present invention.

The present invention provides methods of altering GPCR internalization and identifying compounds which alter GPCR internalization. Preferably, these compounds act by altering PI3K associated GPCR internalization. Preferably, these compounds interfere with the formation of the PI3K/βARK1 complex, the translocation of PI3K to the membrane, the formation of the GPCR/arrestin/adaptin complex, the GPCR/arrestin complex formation, GPCR internalization, or any steps in between.

One embodiment of the present invention is a method of screening compound(s) for modulating G protein-coupled receptor (GPCR) internalization. Most preferably, this method comprises the steps of (a) providing a cell comprising molecules involved in GPCR internalization, wherein the molecules involved in GPCR internalization comprise β adrenergic receptor kinase 1 (βARK1), phosphoinositide 3-kinase (PI3K), GPCR, and arrestin, and wherein at least one of said molecules is detectably labeled; (b) exposing cell to the compound(s); (c) identifying the location in the cell of the labeled molecule; (d) comparing the location of the labeled molecule in the cell in the presence of the compound(s) to the location of the labeled molecule in the cell in the absence of the compound(s); and (e) correlating a difference between (1) the location of the labeled molecule in the cell in the presence of the compound(s) and (2) the location of the labeled molecule in the cell in the absence of the compound(s) to modulation of GPCR internalization.

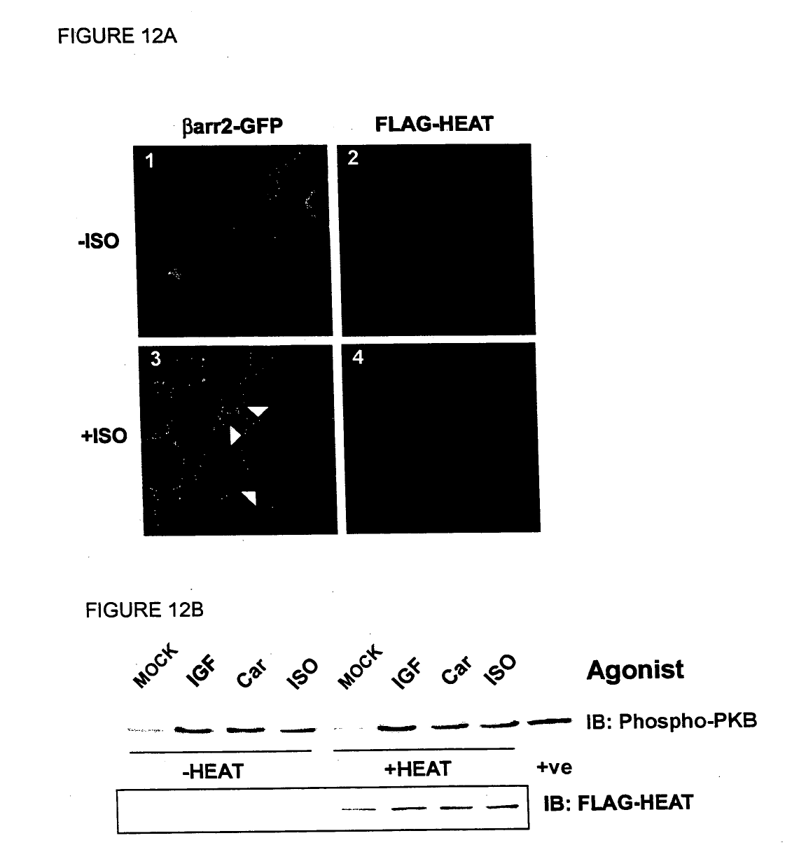

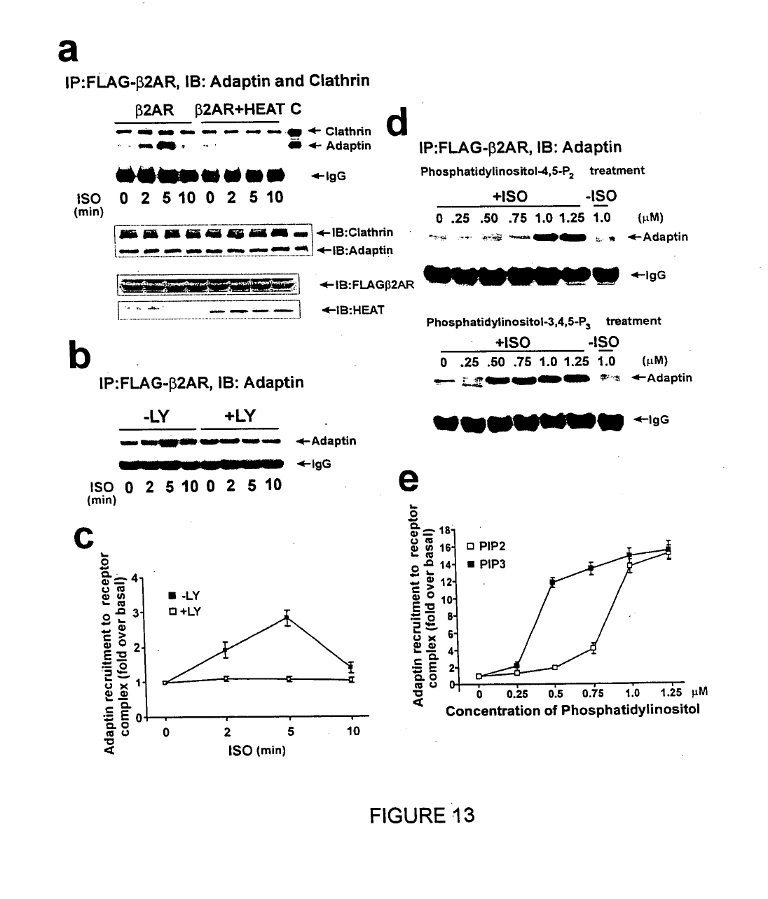

In a preferred embodiment, the GPCR is complexed with one or more other molecules, such as adaptin, as illustrated in FIG. 13. Alternatively or additionally, the GPCR is complexed with arrestin, as illustrated in FIG. 12. The GPCR may or may not directly bind the other molecule.

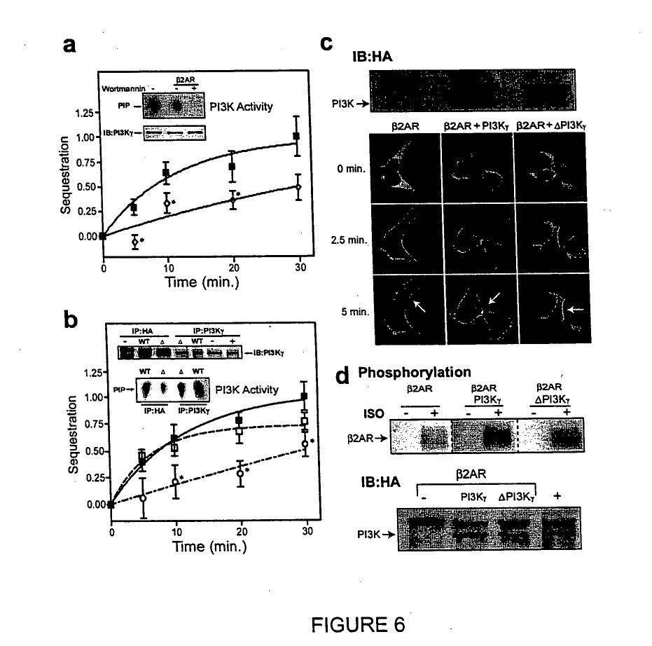

In practicing the method of screening compounds as above, the GPCR/arrestin complex may be labeled. The GPCR may be labeled, as illustrated in FIGS. 6, 11, 14, and 15, or the arrestin may be labeled, as illustrated in FIG. 12. The detection and/or location of members of this complex may indicate that the complex is intact or disrupted, or that the complex is functional or non-functional. Detection of members of this complex indicate the extent to which the GPCR is internalized. Preferably, compounds identified by the present methods decrease the extent to which the GPCR is internalized.

The labeled molecule may then be localized at the cytosol, plasma membrane, endocytic vesicles, or endosomes, and visualized using confocal microscopy, cellular fractionation, and the like, as illustrated in FIGS. 6, 11, 12, 14, and 15.

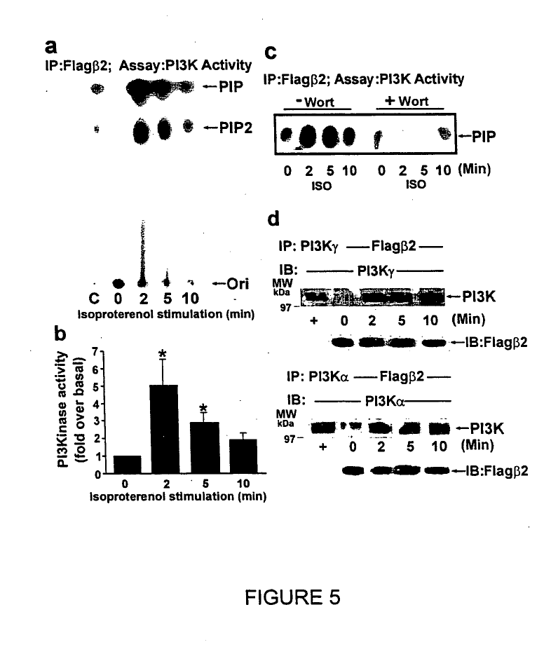

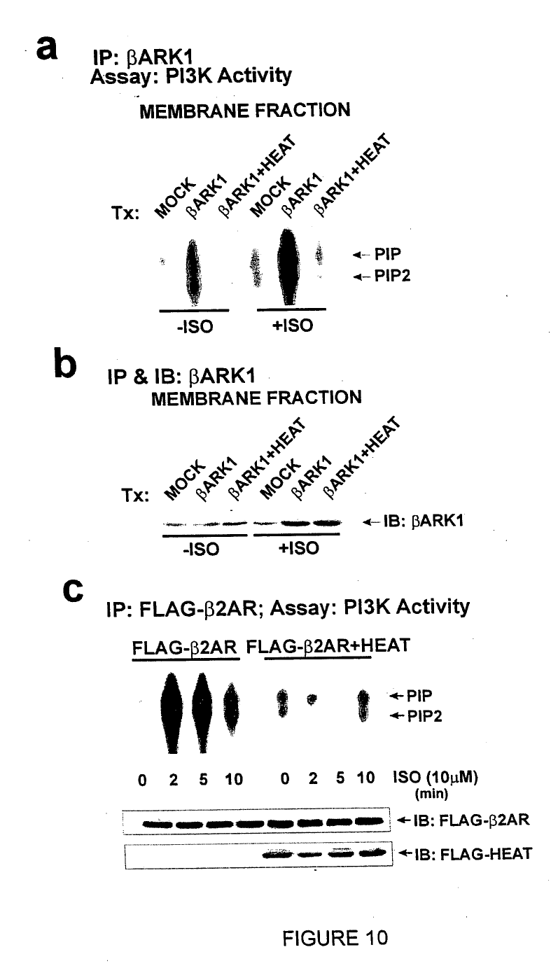

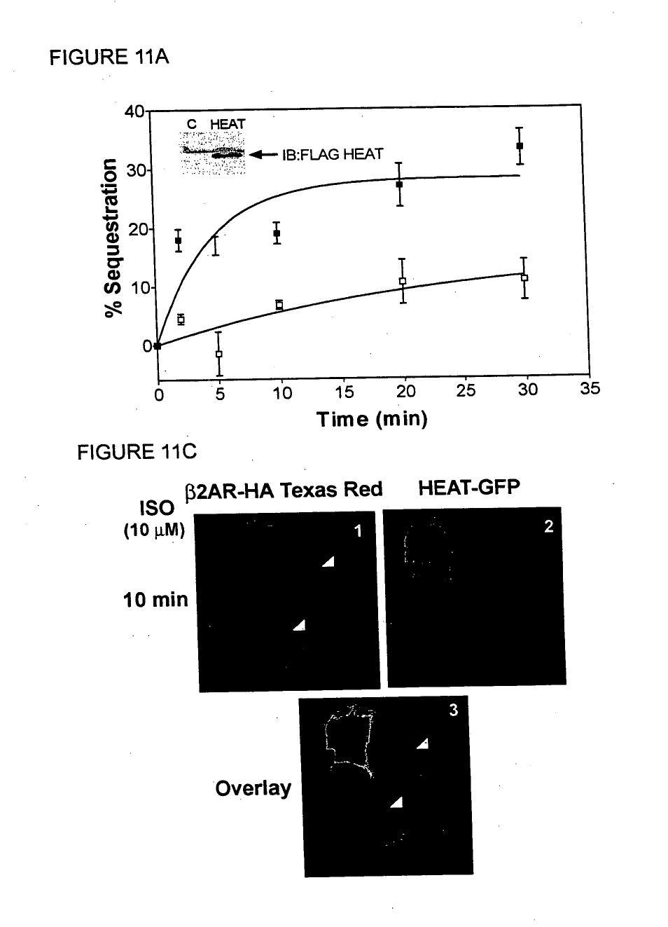

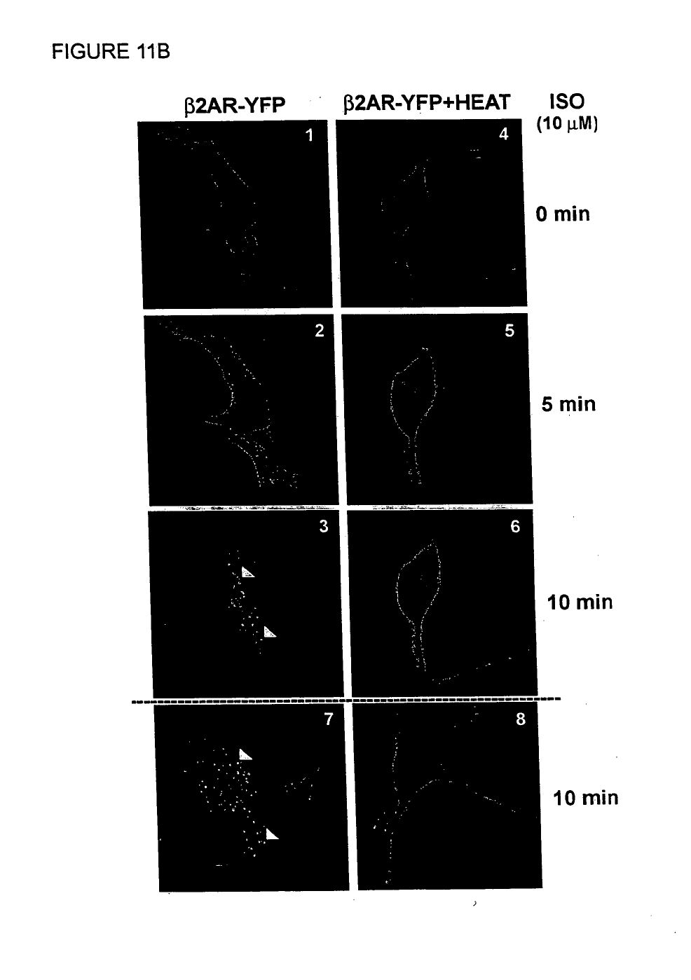

In one aspect of the present invention, the compound alters PI3K catalytic activity, as illustrated in FIGS. 5, 6, and 7, such as altering formation of the PI3K/βARK1 complex, as shown in FIG. 5. In yet another aspect, the compound may inhibit βARK1-mediated translocation of PI3K to the plasma membrane of the cell, as illustrated in FIG. 10. The compounds which alter the above protein functions may alter GPCR internalization, as illustrated in FIGS. 11, 12, 14, and 15.

In accordance with the present screening methods, the location of PI3K within the cell is identified, as illustrated in FIG. 10. The cellular location of PI3K may affect GPCR internalization.

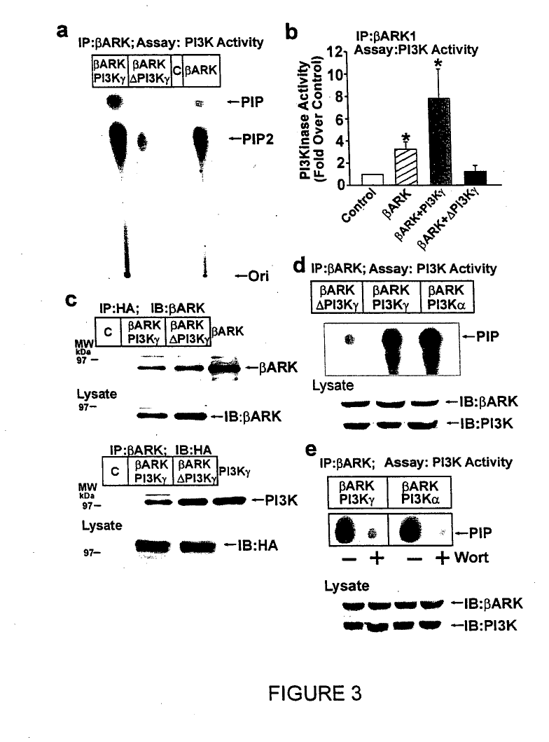

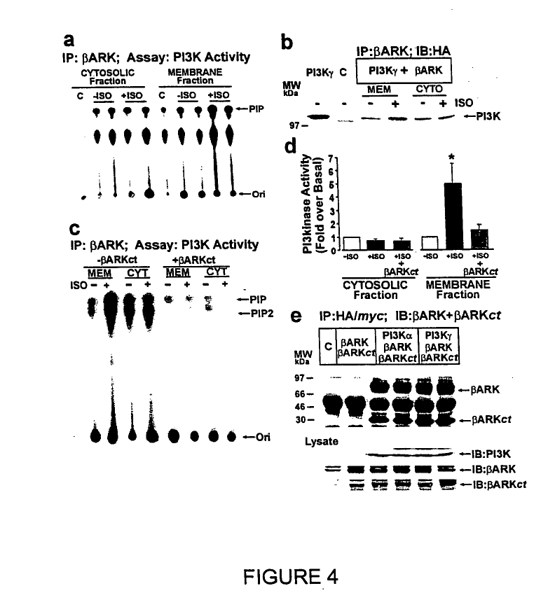

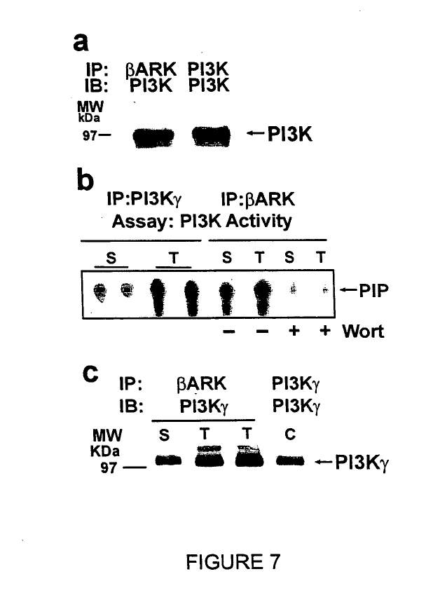

In still another aspect of the present invention, the βARK1 forms a complex with PI3K, as illustrated in FIG. 4. In accordance therewith, the βARK1/PI3K may be labeled, as illustrated in FIG. 4, by either labeling βARK1, as illustrated in FIG. 4, or by labeling the PI3K, as illustrated in FIGS. 3 and 7. The detection and/or location of members of this complex may indicate that the complex is intact or disrupted, or that the complex is functional, or non-functional.

In the-present screening method, the PI3K may be localized in the cytosol or the plasma membrane, as illustrated in FIG. 4.

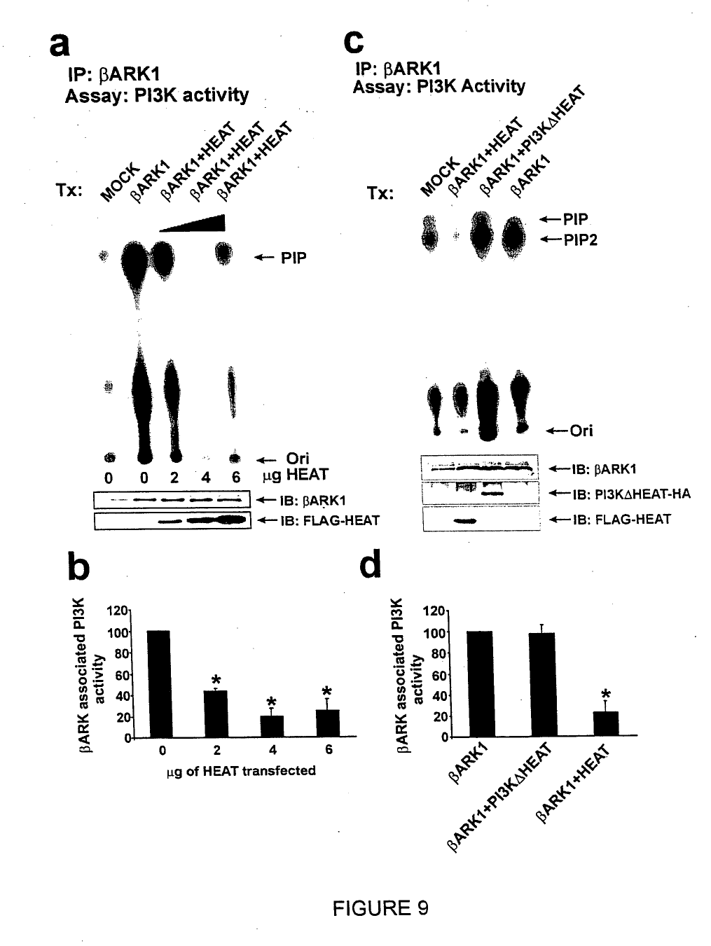

Compounds identified by the present method may inhibit formation of a βARK1/PI3K complex, as illustrated in FIG. 9.

In one embodiment of the present method of screening compounds, the cell further comprises adaptin, which may or may not be labeled, as illustrated in FIG. 13. The detection and/or location of adaptin may indicate that the complex comprising adaptin is intact or disrupted, may indicate that the complex is functional, or non-functional.

Compounds Identified in Above Methods

The methods of the present invention may be used to screen compositions, compounds, sample solutions, chemical libraries, combinatorial libraries, mimetic libraries, immunoglobulins, polypeptides, and the like for antagonists, inverse agonists, agents that interfere with internalization of the GPCR, and the like. By way of example, FIGS. 6, 11, 14, and 15 illustrate compounds interfering with internalization of GPCRs. Likewise, other antagonists and the like could be identified accordingly.

In accordance with another embodiment of the present invention, mutants of PI3K are provided which may have the ability to alter GPCR internalization.

In one preferred embodiment, the PI3K mutants contain only the HEAT domain, which retains the βARK1-binding ability of the wild-type HEAT domain. Another preferred embodiment, the PI3K mutants lack the ability to bind βARK1. Such modified PI3Ks may comprise a mutation of the HEAT domain. The mutation may be an insertion, deletion, or substitution in the HEAT domain. The mutation may instead or additionally be in another portion of the PI3K which results in an altered secondary structure of the molecular interface.

The PI3K mutants which contain only the HEAT domain may retain the ability of the HEAT domain to bind βARK1. Such modified HEAT domains may comprise additional mutations which do not alter the ability of the modified HEAT domain to bind βARK1.

In another embodiment, the PI3K mutant may comprise one or more mutations in other portions of PI3K which result in altered GPCR internalization, such as by altering PI3K catalytic activity. Such mutants may retain ability to bind P3ARK1, but lack or have decreased PI3K catalytic activity. Another embodiment is a PI3K mutant which lacks the ability to translocate to the membrane.

In accordance with yet another embodiment of the present invention, βARK1 mutants are provided. Certain βARK1 mutants may have altered PI3K binding. In a particular embodiment, the βARK1 mutants are modified in the domain that directly binds PI3K. In another particular embodiment, the βARK1 mutants are not modified in the domain that directly binds P3ARK1, but such mutants interfere with PI3K binding βARK1. βARK1 has an extended PH domain involved in protein-protein interactions. In a particular embodiment, modifications of this domain alter PI3K binding.

Other modified polypeptides and peptidomimetics in the desensitization pathway will be identified in the above methods. Such compounds include adaptin mutants and arrestin mutants.

Mutations can be made in the polypeptides described above such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

Identified compounds may inhibit GPCR internalization by modification of the βARK1/PI3K complex, PI3K catalytic activity, PI3K translocation to the membrane, formation of the GPCR/arrestin/adaptin complex, GPCR internalization, any steps between, or the like.

Disease Treatment

Another aspect of the invention relates to methods of treating a human or non-human subject suffering from a GPCR-related disease, such as cardiovascular disease, heart failure, asthma, nephrogenic diabetes insipidus, or hypertension. Such treatment can be performed either by administering to a subject in need of such treatment, an amount of the agonists or antagonists identified by the present method sufficient to treat the GPCR-related disease, or at least to lessen the symptoms thereof.

Treatment may also be effected by administering to the subject the naked modified nucleic acid sequences of the invention, such as by direct injection, microprojectile bombardment, delivery via liposomes or other vesicles, or by means of a vector which can be administered by one of the foregoing methods. Gene delivery in this manner may be considered gene therapy. Preferably, the naked modified nucleic acid sequences comprise modified PI3Ks, modified HEAT domains, or modified βARK1 proteins of the present invention.

Expression of the Modified Proteins

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, plant cells, nematode cells, and animal cells, such as HEK-293, CHO, RI.I, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that modified PI3K analogs may be prepared from nucleotide sequences of the protein complex/subunit derived within the scope of the present invention. Analogs, such as fragments, may be produced, for example, by pepsin digestion of PI3K material. Other analogs, such as muteins, can be produced by standard site-directed mutagenesis of PI3K coding sequences. Analogs exhibiting “PI3K activity” such as small molecules, whether functioning as promoters or inhibitors, may be identified by known in vivo and/or in vitro assays.

As mentioned above, a DNA sequence encoding a modified PI3K can be prepared synthetically rather than cloned. The DNA sequence can be designed with the appropriate codons for the PI3K amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient-construction of genes which will express PI3K analogs or “muteins”. Alternatively, DNA encoding muteins can be made by site-directed mutagenesis of native or modified PI3K genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

Antisense

The present invention extends to the preparation of antisense oligonucleotides and ribozymes that may be used to interfere with the expression of a PI3K, βARK1, and the like at the translational level. Preferably, the antisense and ribozymes may be used to interfere with the expression of a PI3K, βARK1, and the like having discrete point mutations that increases its affinity for arrestin in suspect target cells. This approach utilizes antisense nucleic acid and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule. (See Weintraub, Sci Am. 1990 January; 262(1):40-6; Marcus-Sekura, Anal Biochem. 1988 Aug. 1; 172(2):289-95). In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into cells. Antisense methods have been used to inhibit the expression of many genes in vitro (Marcus-Sekura, 1988; Hambor et al., J Exp Med. 1988 Oct. 1; 168(4):1237-45).

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, Gene. 1988 Dec. 20; 73(2):259-71). Because they are sequence-specific, only mRNAs with particular sequences are inactivated.

Investigators have identified two types of ribozymes, Tetrahymena-type and “hammerhead”-type. (Hasselhoff and Gerlach, 1988) Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteenbase recognition sequences are preferable to shorter recognition sequences.

The DNA sequences described herein may thus be used to prepare antisense molecules against, and ribozymes that cleave mRNAs for PI3Ks and their ligands. In particular, the antisense molecules and ribozymes may be particularly useful for PI3Ks having mutations that alter their affinity for GRKs.

Antibodies

Also, antibodies including both polyclonal and monoclonal antibodies, and drugs that modulate the production or activity of the βARK1/PI3K complex and/or their biologically active fragments or subunits may possess certain diagnostic or therapeutic applications. For example, the βARK1/PI3K complex or fragments or subunits thereof may be used to produce both polyclonal and monoclonal antibodies, to the βARK1/PI3K complex or fragments or subunits thereof, in a variety of cellular media, by known techniques such as the hybridoma technique utilizing, for example, fused mouse spleen lymphocytes and myeloma cells. Likewise, small molecules that mimic or antagonize the activity(ies) of the βARK1/PI3K complex of the invention may be discovered or synthesized, and may be used in diagnostic and/or therapeutic protocols.

The present invention likewise extends to the development of antibodies against the βARK1/PI3K complex, including naturally raised and recombinantly prepared antibodies. For example, the antibodies could be used to screen expression libraries to obtain the gene or genes that encode subunits of the βARK1/PI3K complex. Such antibodies could include both polyclonal and monoclonal antibodies prepared by known genetic techniques, as well as bi-specific (chimeric) antibodies, and antibodies including other functionalities suiting them for additional diagnostic use conjunctive with their capability of modulating βARK1/PI3K complex activity. Preferably, the anti-βARK1/PI3K complex antibody used in the diagnostic methods of this invention is an affinity purified polyclonal antibody. More preferably, the antibody is a monoclonal antibody (mAb). In addition, it is preferable for the anti-modified-GPCR antibody fragments used herein be in the form of Fab, Fab′, F(ab′)₂, F(v), or scFv.

The general methodology for making monoclonal antibodies by hybridomas is well known. Methods for producing monoclonal anti-GPCR antibodies are also well-known in the art. See Niman et al., Proc. Natl. Acad. Sci. USA, 80:4949-4953 (1983). Typically, the βARK1/PI3K complex or a peptide analog is used either alone or conjugated to an immunogenic carrier, as the immunogen in the before described procedure for producing anti-βARK1/PI3K complex monoclonal antibodies. The culture is maintained under conditions and for a time period sufficient for the hybridoma to secrete the antibodies into the medium. The hybridomas are screened for the ability to produce an antibody that immunoreacts with the βARK1/PI3K complex or peptide analog. The antibody-containing medium is then collected. The antibody can then be further isolated by well-known techniques. Immortal, antibody-producing cell lines can also be created by techniques other than fusion, such as direct transformation of B lymphocytes with oncogenic DNA, or transfection with Epstein-Barr virus. See, e.g., Using Antibodies: A Laboratory Manual, Harlow, Ed and Lane, David (Cold Spring Harbor Press, 1999).

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mM glutamine, and 20% fetal calf serum. A preferred inbred mouse strain is the Balb/c.

Methods for producing polyclonal anti-polypeptide antibodies are well-known in the art. See U.S. Pat. No. 4,493,795 to Nestor et al. A monoclonal antibody, and immunologically active fragments thereof, can be prepared using the hybridoma technology described in Using Antibodies: A Laboratory Manual, Harlow, Ed and Lane, David (Cold Spring Harbor Press, 1999), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a βARK1/PI3K complex. Splenocytes are typically fused with myeloma cells using polyethylene glycol (PEG) 6000 MW. Fused hybrids are selected by their sensitivity to HAT (hypoxanthine, aminopterin, thymidine) supplemented media. Hybridomas producing a monoclonal antibody useful in practicing this invention are identified by their ability to immunoreact with the present βARK1/PI3K complex and their ability to inhibit specified βARK1/PI3K complex activity in target cells.

In particular, antibodies against specifically phosphorylated factors can be selected and are included within the scope of the present invention for their particular ability in following activated protein. Thus, activity of the modified βARK1/PI3K complex or of the specific polypeptides believed to be causally connected thereto may therefore be followed directly by the assay techniques discussed herein, through the use of an appropriately labeled quantity of the βARK1/PI3K complex or antibodies or analogs thereof.

Panels of monoclonal antibodies produced against βARK1/PI3K complex peptides can be screened for various properties; i.e., isotype, epitope, affinity, and the like. Of particular interest are monoclonal antibodies that neutralize the activity of the βARK1/PI3K complex or its subunits. Such monoclonals can be readily identified in βARK1/PI3K complex assays. High affinity antibodies are also useful when immunoaffinity purification of native or recombinant βARK1/PI3K complex is possible.

Thus, the βARK1/PI3K complex, its analogs and/or analogs, and any antagonists or antibodies that may be raised thereto, are capable of use in connection with various diagnostic techniques, including immunoassays, such as a radioimmunoassay, using for example, an antibody to the βARK1/PI3K complex that has been labeled by either radioactive addition, or radioiodination.

In an immunoassay, a control quantity of the antagonists or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached. For example, antibodies against specifically phosphorylated factors may be selected and appropriately employed in the exemplary assay protocol, for the purpose of following activated protein as described above.

In the instance where a radioactive label, such as, but not limited to, the isotopes ³H, ¹⁴C, ³²P, ³⁵S, ³⁶Cl, ⁵¹Cr, ⁵⁷Co, ⁵⁸Co; ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques discussed herein and as known in the art.

As suggested earlier, the diagnostic method of the present invention comprises examining a cellular sample or medium by means of an assay including an effective amount of an antagonist to a modified GPCR/protein, such as an anti-βARK1/PI3K complex antibody, preferably an affinity-purified polyclonal antibody, and more preferably a mAb. In addition, it is preferable for the anti-modified-GPCR antibody fragments used herein to be in the form of Fab, Fab′, F(ab′)₂, F(v), or scFv. As previously discussed, patients capable of benefitting from this method include those suffering from cancer, a pre-cancerous lesion, a viral infection or other like pathological condition or disease. Methods for isolating the βARK1/PI3K complex, inducing anti-βARK1/PI3K complex antibodies, and determining and optimizing the ability of anti-βARK1/PI3K complex antibodies to assist in the examination of the target cells are all well-known in the art.

The present invention includes an assay system which may be prepared in the form of a test kit for the quantitative analysis of the extent of the presence of the βARK1/PI3K complex, or to identify drugs or other agents that may mimic or block their activity. The system or test kit may comprise a labeled component prepared by one of the radioactive and/or enzymatic techniques discussed herein, coupling a label to the βARK1/PI3K complex, their agonists and/or antagonists, and one or more additional immunochemical reagents, at least one of which is a free or immobilized ligand, capable either of binding with the labeled component, its binding partner, one of the components to be determined or their binding, partner(s).

Conjugates

The cells used in the methods of assaying of the present invention may comprise a conjugate of an arrestin protein and a detectable molecule, a conjugate of a GPCR and a detectable molecule, a conjugate of any member of a GPCR/arrestin complex and a detectable molecule, a conjugate of any member of a GPCR/arrestin/adaptin complex and a detectable molecule, a conjugate of βARK1 and a detectable molecule, a conjugate of PI3K and a detectable molecule, a conjugate of any member of a βARK1/PI3K complex and a detectable molecule, a conjugate of a HEAT domain and a detectable molecule, and the like. The detectable molecule allows detection of molecules interacting with the detectable molecule, as well as the molecule itself.

All forms of arrestin, naturally occurring and engineered variants, including but not limited to, visual arrestin, β-arrestin 1 and β-arrestin 2, may be used in the present invention. GPCRs may interact to a detectable level with all forms of arrestin.

Detectable molecules that may be used include, but are not limited to, molecules that are detectable by spectroscopic, photochemical, biochemical., immunochemical, electrical, radioactive, and optical means, including but not limited to bioluminescence, phosphorescence, and fluorescence. These detectable molecules should be a biologically compatible molecule and should not compromise the biological function of the molecule and must not compromise the ability of the detectable molecule to be detected. Preferred detectable molecules are optically detectable molecules, including optically detectable proteins, such that they may be excited chemically, mechanically, electrically, or radioactively to emit fluorescence, phosphorescence, or bioluminescence. More preferred detectable molecules are inherently fluorescent molecules, such as fluorescent proteins, including, for example, Green Fluorescent Protein (GFP). The detectable molecule may be conjugated to the arrestin protein by methods as described in Barak et al. (U.S. Pat. Nos. 5,891,646 and 6,110,693). The-detectable molecule may be conjugated at the front-end, at the back-end, or in the middle.

The GPCRs may also be conjugated with a detectable molecule. Preferably, the carboxyl-terminus of the GPCR is conjugated with a detectable molecule. If the GPCR is conjugated with a detectable molecule, proximity of the GPCR with the arrestin may be readily detected. In addition, if the GPCR is conjugated with a detectable molecule, compartmentalization of the GPCR with the arrestin may be readily confirmed. The detectable molecule used to conjugate with the GPCRs may include those as described above, including, for example, optically detectable molecules, such that they may be excited chemically, mechanically, electrically, or radioactively to emit fluorescence, phosphorescence, or bioluminescence. Preferred optically detectable molecules may be detected by immunofluorescence, luminescence, fluorescence, and phosphorescence.

For example, the GPCRs may be antibody labeled with an antibody conjugated to an immunofluorescence molecule or the GPCRs may be conjugated with a luminescent donor. In particular, the GPCRs may be conjugated with, for example, luciferase, for example, Renilla luciferase, or a rhodamine-conjugated antibody, for example, rhodamine-conjugated anti-HA mouse monoclonal antibody. Preferably, the carboxyl-terminal tail of the GPCR may be conjugated with a luminescent donor, for example, luciferase. The GPCR, preferably the carboxyl-terminal tail, also may a be conjugated with GFP as described in L. S. Barak et al. Internal Trafficking and Surface Mobility of a Functionally Intact β₂-Adrenergic Receptor-Green Fluorescent Protein Conjugate, Mol. Pharm. (1997) 51, 177-184.

Cell Types and Substrates

The cells of the present invention express at least one βARK1, PI3K, GPCR, and arrestin, wherein at least one of the molecules is detectably labeled. Cells useful in the present invention include eukaryotic and prokaryotic cells, including, but not limited to, bacterial cells, yeast cells, fungal cells, insect cells, nematode cells, plant cells, and animal cells. Suitable animal cells include, but are not limited to, HEK cells, HeLa cells, COS cells, and various primary mammalian cells. An animal model expressing a conjugate of an arrestin and a detectable molecule throughout its tissues or within a particular organ or tissue type, may also be used in the present invention.

A substrate may have deposited thereon a plurality of cells of the present invention. The substrate may be any suitable biologically substrate, including but not limited to, glass, plastic, ceramic, semiconductor, silica, fiber optic, diamond, biocompatible monomer, or biocompatible polymer materials.

Methods of Detection

Methods of detecting the intracellular location of the detectably labeled arrestin, the intracellular location of a detectably labeled GPCR, or interaction of the detectably labeled adaptin, arrestin, or other member of GPCR/arrestin/adaptin complex with a GPCR or any other cell structure, including for example, the concentration of arrestin, adaptin, or GPCR at a cell membrane, colocalization of arrestin with GPCR in endosomes, and concentration of arrestin, adaptin, or GPCR in clathrin-coated pits, and the like, will vary dependent upon the detectable molecule(s) used. Methods of detecting the intracellular location of the detectably labeled PI3K, the intracellular location of a detectably labeled βARK1, or interaction of the detectably labeled member of βARK1/PI3K complex with any other cell structure, including for example, the concentration of PI3K or βARK1 at a cell membrane, colocalization of PI3K with P3ARK1, and the like, will vary dependent upon the detectable molecule(s) used.

One skilled in the art readily will be able to devise detection methods suitable for the detectable molecule(s) used. For optically detectable molecules, any optical method may be used where a change in the fluorescence, bioluminescence, or phosphorescence may be measured due to a redistribution or reorientation of emitted light. Such methods include, for example, polarization microscopy, BRET, FRET, evanescent wave excitation microscopy, and standard or confocal microscopy.

In a preferred embodiment arrestin may be conjugated to GFP and the arrestin-GFP conjugate may be detected by confocal microscopy. In another preferred embodiment, arrestin may conjugated to a GFP and the GPCR may be conjugated to an immunofluorescent molecule, and the conjugates may be detected by confocal microscopy. In an additional preferred embodiment, arrestin may conjugated to a GFP and the carboxy-terminus of the GPCR may be conjugated to a luciferase and the conjugates may be detected by bioluminescence resonance emission technology. In a further preferred embodiment arrestin may be conjugated to a luciferase and GPCR may be conjugated to a GFP, and the conjugates may be detected by bioluminescence resonance emission technology. The methods of the present invention are directed to detecting GPCR activity. The methods of the present invention allow enhanced monitoring of the GPCR pathway in real time.

In a preferred embodiment, the localization pattern of the detectable molecule is determined. In a further preferred embodiment, alterations of the localization pattern of the detectable molecule may be determined. The localization pattern may indicated cellular localization of the detectable molecule. Certain methods of detection are described in U.S. patent application Ser. No. ______ (Atty Docket No: 033072-030), filed Mar. 12, 2002, which claims priority to U.S. Provisional Patent Application No. 60/275,339, filed Mar. 13, 2001, the contents of which are incorporated by reference in their entirety.

The biological activities of the molecules themselves may be detected, such as the catalytic activity of PI3K, and the like. Molecules may also be detected by their interaction with another detectably labeled molecule, such as an antibody.

Pharmaceutical Compositions

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A GPCR agonist, antagonist, compound, or DAC obtained by the methods disclosed herein can be formulated into the therapeutic composition as neutralized pharmaceutically acceptable salt forms. Pharmaceutically acceptable salts include the acid addition salts (formed with the free amino groups of the polypeptide or antibody) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent (i.e., carrier, or vehicle).

The data obtained from the cell culture assays and animal studies can be used in formulating a range of dosage for use in humans. The dosage of such compositions lies preferably within a range of circulating concentrations that include the ED50 with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range which includes the IC50 (i.e., the concentration of the test composition which achieves a half-maximal inhibition of symptoms) as determined in cell culture. Such information can be used to more accurately determine useful doses in humans. Levels in plasma may be measured, for example, by high performance liquid chromatography.

The compositions are administered in a manner compatible with the dosage formulation, and in a therapeutically effective amount. The quantity to be administered depends on the subject to be treated, capacity of the subject's immune system to utilize the active ingredient, and degree of modulation of GPCR activity desired. Precise amounts of active ingredient required to be administered depend on the judgment of the practitioner and are peculiar to each individual. However, suitable dosages may range from about 0.001 to 30, preferably about 0.01 to about 25, and more preferably about 0.1 to 20 milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The skilled artisan will appreciate that certain factors may influence the dosage required to effectively treat a subject, including but not limited to, the severity of the disease or condition, disorder, or disease, previous treatments, the general health and/or age of the subject, and other diseases present. Moreover, treatment of a subject with a therapeutically effective amount of the composition(s) can include a single treatment or, preferably, can include a series of treatments. In a preferred example, a subject is treated with the composition in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of the composition used for treatment may increase or decrease over the course of a particular treatment. Changes in dosage may result and become apparent from the results of diagnostic assays as described herein.

The therapeutic compositions may further include an effective amount of the GPCR agonist, antagonist, or DAC and one or more of the following active ingredients: an antibiotic, a steroid, and the like.

The term “prodrug” indicates a therapeutic agent that is prepared in an inactive form that is converted to an active-form (i.e., drug) within the body or cells thereof by the action of endogenous enzymes or other chemicals and/or conditions. In particular, prodrug versions of the oligonucleotides of the invention can be prepared as SATE ((S-acetyl-2-thioethyl) phosphate) derivatives according to the methods disclosed for example in WO 93/24510 and in WO 94/26764.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. The compounds for modulating any of the disclosed genes, gene transcripts or proteins encoded thereby include antisense compounds as well as other modulatory compounds.

Pharmaceutically acceptable base addition salts for use with antisense as well as other modulatory compounds are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., “Pharmaceutical Salts,” J. Pharma. Sci., 1977, 66: 1-19). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are known in the art and include basic salts of a variety of inorganic and organic acids, such as, for example, with inorganic acids (e.g., hydrochloric acid, hydrobromic acid, sulfuric acid or phosphoric acid); with organic carboxylic, sulfonic, sulfo- or phospho-acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid.

Pharmaceutically acceptable salts of compounds may also be prepared with a pharmaceutically acceptable cation. Suitable pharmaceutically acceptable cations are well known in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds and other modulatory compounds described herein can be utilized in pharmaceutical compositions by adding an effective amount of an antisense compound or other modulatory compound to a suitable pharmaceutically acceptable diluent or carrier. Use of the compounds and methods of the invention may also be useful prophylactically.

The antisense compounds of the invention are useful for research and diagnostics, because these compounds hybridize to nucleic acids encoding a gene identified using the systematic discovery technique or a mRNA transcript thereof. Such hybridization allows the use of sandwich and other assays to easily be constructed to exploit this fact. Hybridization of the antisense oligonucleotides of the invention with a nucleic acid encoding a gene or gene transcript identified by a systematic discovery method can be detected by means known in the art. Such means may include, for example, conjugation of an enzyme to the oligonucleotide, radiolabelling of the oligonucleotide or any other suitable detection means. Kits using such detection means for detecting the level of a transcript of a gene in a sample may also be prepared.

The present invention also includes pharmaceutical antisense compositions and formulations which include the antisense compounds and other modulatory compounds and compositions of the invention. The pharmaceutical compositions of the present invention may be administered in a number of ways depending upon whether local or systemic treatment is desired and upon the area to be treated.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compositions may be combined with a carrier so that an effective dosage is delivered, based on the desired activity.

Pharmaceutical compositions and formulations for topicaI administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato-starch or sodium starch glycolate); or wetting agents (e.g., sodium lauryl sulphate). The tablets may be coated by methods well known in the-art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer, salts, flavoring, coloring and sweetening agents as appropriate.

Preparations for oral administration may be suitably formulated to give controlled release of the active composition.

The compositions may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For administration by inhalation, the compositions for use according to the present invention are conveniently delivered in the form of an aerosol spray, presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the composition and a suitable powder base such as lactose or starch.

The compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pharmaceutical compositions (e.g., gene, gene transcript or protein product modulatory agents as described herein) of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In one embodiment of the present invention, the pharmaceutical compositions may be formulated and used as foams. Pharmaceutical foams include formulations such as, but not limited to, emulsions, microemulsions, creams, jellies and liposomes. While basically similar in nature, these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 m in diameter. See, e.g., Idson, in Pharmaceutical Dosage Forms v. 1, p. 199 (Lieberman, Rieger and Banker (Eds.), 1988, Marcel Dekker, Inc., New York); Rosoff, in Pharmaceutical Dosage Forms, v. 1, p. 245; Block in Pharmaceutical Dosage Forms, v. 2, p. 335; Higuchi et al., in Remington's Pharmaceutical Sciences 301 (Mack Publishing Co., Easton, Pa., 1985). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms v. 1, p. 199 (Lieberman, Rieger and Banker (Eds.), 1988, Marcel Dekker, Inc., New York).

Synthetic surfactants, also known as surface active agents, have found wide applicability in the formulation of emulsions and have been reviewed in the literature (Rieger, in Pharmaceutical Dosage Forms, v. 1, p. 285; Idson, in Pharmaceutical Dosage Forms, v. 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers, especially in combination with surfactants and in viscous preparations. These include polar inorganic solids, such as heavy metal hydroxides, non-swelling clays (e.g., bentonite, aftapulgite, hectorite, kaolin, montmorillonite, colloidal aluminum silicate and colloidal magnesium aluminum silicate), pigments and nonpolar solids (e.g., carbon or glyceryl tristearate).

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, v. 1 p. 385 (Lieberman, Rieger and Banker (Eds.), 1988, Marcel Dekker, Inc., New York)).

Hydrophilic colloids or hydrocolloids include naturally occurring gums and synthetic polymers, such as polysaccharides (e.g., acacia, agar, alginic acid, carrageenan, guar gum, karaya gum, and tragacanth), cellulose derivatives (e.g., carboxymethylcellulose and carboxypropylcellulose), and synthetic polymers (e.g., carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers (e.g., tocopherols, alkyl gallates, butylated hydroxyanisole, butylated hydroxytoluene) or reducing agents (e.g., ascorbic acid and sodium metabisulfite), and antioxidant synergists (e.g., citric acid, tartaric acid, and lecithin).

The application of emulsion formulations via dermatological, oral and parenteral routes and methods for their manufacture have been reviewed in the literature (Idson, in Pharmaceutical Dosage Forms, v. 1, p. 199). Emulsion formulations for oral delivery have been very widely used because of reasons of ease of formulation, efficacy from an absorption and bioavailability standpoint. (Rosoff, in Pharmaceutical Dosage Forms, v. 1, p. 245 (Lieberman, Rieger and Banker (Eds.), 1988, Marcel Dekker, Inc., New York); Idson, in Pharmaceutical Dosage Forms). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, v. 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in Controlled Release of Drugs: Polymers and Aggregate Systems, 185-215 (Rosoff, M., Ed., 1989, VCH Publishers, New York). Microemulsions commonly are prepared via a combination of three to five components-that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, 271 (Mack Publishing Co., Easton, Pa., 1985).

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with co-surfactants. The co-surfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of co-surfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono-, di-, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharm. Res., 1994, 11:1385-90; Ritschel, Meth. Find. Exp. Clin. Pharmacol., 1993, 13: 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., 1994; Ho et al., J. Pharm. Sci., 1996, 85: 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids and other active agents from the gastrointestinal tract, as well as improve the local cellular uptake of oligonucleotides and nucleic acids and other active agents within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Crit. Rev. Therap. Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, are useful because of their specificity and the duration of action. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. Selection of the appropriate liposome depending on the agent to be encapsulated would be evident given what is known in the art.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: (a) liposomes obtained from natural phospholipids are biocompatible and biodegradable; (b) liposomes can incorporate a wide range of water and lipid soluble drugs; (c) liposomes can protect encapsulated drugs in their internal compartments from metabolism and degradation (Rosoff, in Pharmaceutical Dosage Forms). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Another embodiment also contemplates the use of liposomes for topical administration. Such advantages include reduced side-effects related to high systemic absorption of the administered drug, increased accumulation of the administered drug at the desired target, and the ability to administer a wide variety of drugs, both hydrophilic and hydrophobic, into the skin. Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Comm., 1987,147:980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., J. Controlled Release, 1992,19: 269-74).

Another contemplated liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

“Sterically stabilized” liposomes, which refers to liposomes comprising one or more specialized lipids that, when incorporated into liposomes, result in enhanced circulation lifetimes relative to liposomes lacking such specialized lipids are also contemplated. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Left., 1987, 223: 42; Wu et al., Can. Res., 1993, 53: 3765).

Many liposomes comprising lipids derivatized with one or more hydrophilic polymers, and methods of preparation thereof, are known in the art. See, e.g., Sunamoto et al. (Bull. Chem. Soc. Jpn., 1980, 53: 2778) described liposomes comprising a nonionic detergent, 2C12 15G, that contains a PEG moiety. Illum et al. (FEBS Lett., 1984, 167: 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268: 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029: 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by, e.g., Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.). Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

Methods of encapsulating nucleic acids in liposomes is also known in the art. See, WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, p. 285 (Marcel Dekker, Inc., New York, N.Y., 1988, p. 285)).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, 285 (Marcel Dekker, Inc., New York, N.Y., 1988).

In one embodiment, the present invention employs various penetration enhancers to effect the efficient delivery of nucleic acids and other agents, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Another embodiment of the invention contemplates pharmaceutical compositions comprising surfactants. Surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Crit. Rev. Therap. Drug Carrier Systems, 1991, 92); and perfluorochemical emulsions, such as FC-43 (Takahashi et al., J. Pharm. Pharmacol., 1988, 40: 252).

Another embodiment contemplates the use of various fatty acids and their derivatives to act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C1-10 alkyl esters thereof (e.g., methyl, isopropyl and t-butyl), and mono- and di-glycerides thereof (i.e., oleate, laurate, caprate, myristate, palmitate, stearate, linoleate, and the like) (Lee et al., 1991; Muranishi, Crit. Rev. Therap. Drug Carrier Systems, 1990, 7: 1-33; El Hariri et al., J. Pharm. Pharmacol., 1992, 44: 651-4).

The compositions comprising the active agents of the invention may further comprise bile salts. The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, N.Y., 1996, pp. 934-935). Various natural bile salts, and their synthetic derivatives, act as penetration enhancers. Thus, the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., 1991; Swinyard, Chapter 39 In: Remington's Pharmaceutical Sciences, 18th Ed., Gennaro, ed., Mack Publishing Co., Easton, Pa., 1990, pages 782-783; Muranishi, 1990; Yamamoto et al., J. Pharm. Exp. Ther., 1992, 263: 25; Yamashita et al., J. Pharm. Sci., 1990, 79: 579-83).

The invention further contemplates compositions comprising chelating agents; Chelating agents can be defined as compounds that remove metallic ions from solution by forming complexes therewith, with the result that absorption of oligonucleotides through the mucosa is enhanced. With regards to their use as penetration enhancers for use when the active agent is an antisense agent, chelating agents have the added advantage of also serving as DNase inhibitors, as most characterized DNA nucleases require a divalent metal ion for catalysis and are thus inhibited by chelating agents (Jarrett, J. Chromatogr., 1993, 618: 315-39). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., 1991; Muranishi, 1990; Buur et al., J. Control Rel., 1990, 14: 43-51).

The invention also contemplates pharmaceutical compositions comprising active agents and non-chelating non-surfactants. Non-chelating non-surfactant penetration enhancing compounds can be defined as compounds that demonstrate insignificant activity as chelating agents or as surfactants, but that nonetheless enhance absorption of oligonucleotides through the alimentary mucosa (Muranishi, 1990). This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., J. Pharm. Pharmacol., 1987, 39: 621-6).

For pharmaceutical compositions comprising oligonucleotides, agents that enhance uptake of oligonucleotides at the cellular level may also be added to the pharmaceutical and other compositions of the present invention. For example, cationic lipids, such as lipofectin (Junichi et al., U.S. Pat. No. 5,705,188), cationic glycerol derivatives, and polycationic molecules, such as polylysine (Lollo et al.,. PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes (e.g., limonene and menthone).

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 1995, 5: 115-121; Takakura et al., Antisense & Nucl. Acid Drug Dev., 1996, 6: 177-183).

The pharmaceutical compositions disclosed herein may also comprise a excipients. In contrast to carrier compounds described above, these excipients include a pharmaceutically acceptable solvent, suspending agent or any other pharmacologically inert vehicle for delivering one or more nucleic acids or other active agents to an animal. The excipient may be liquid or solid and is selected, with the planned manner of administration in mind, so as to provide for the desired bulk, consistency, etc., when combined with a nucleic acid or other active agent and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids and other contemplated active agents may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids or other contemplated active agents can be used.

The compositions of the present invention may additionally contain other adjunct components conventionally found in pharmaceutical compositions, at their art-established usage levels. Thus, for example, the compositions may contain additional, compatible, pharmaceutically-active materials such as, e.g., antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

In another related embodiment, compositions of the invention may contain one or more antisense compound or other active agents. Two or more combined compounds may be used together or sequentially.

### EXAMPLES

Cell Culture—Mouse NIH-3T3 and HEK293 cells were maintained in either Iscoves modified Dulbecco's medium or minimal essential medium (MEM) supplemented with 10% fetal bovine serum and 1:100 penicillin-streptomycin (10,000 U/ml) at 37° C. Cells were seeded at a density of ˜1-3×10⁵ cells/35 mm² dish and at 70-80% confluence were transiently transfected using the transfection reagent FUGENE6 (ROCHE) or calcium phosphate precipitation. Cells were harvested 24 hours after transfection, re-plated in triplicate, allowed to grow overnight and serum starved for 2-4 hours before agonist stimulation. Cell lines with stable expression of FLAG-β₂AR (a gift from Dr. Robert Lefkowitz) and HA-β₂AR were used for the confocal experiments. HA-β₂AR and β-arrestin double stable cell lines were prepared by selecting the cells against two different antibiotics.

Plasmid Constructs—The cDNA encoding bovine βARK1 and the carboxyl terminus of βARK1 (βARKct) was described previously. Genomic DNA isolated from mouse tail was used for amplification of β₁AR gene by polymerase chain reaction with Pfu Taq polymerase (Stratagene) using the 5′ primer (5′AATTCgCCgCCATGgACTACAAggACgACgATgATAAgggCgCgggggCgC TCgCCCTg 3′) containing an EcoRI site for subcloning followed by Kozak consensus sequence and a FLAG tag and 3′ primer (5′MGCTTCTACTTGGACTCCGAGGA 3′) containing consensus stop codon with a HindIII site for subcloning. Pfu Taq polymerase amplified PCR product was subcloned in zero-blunt TOPO-vector (Invitrogen) and was cut with EcoRI/HindIII and subcloned into pRK5 mammalian expression vector. The cloned β₁AR in pRK5 was then sequenced to check for its authenticity. FLAG tagged β₂AR was a generous gift from Dr. Robert J. Lefkowitz. HA tagged pCMV-PI3 Kp110γ wild type (PI3Kγ), HA tagged pCMV-PI3 Kp110γ mutant (ΔPI3Kγ) (Δ942-981, deletion in ATP binding site) were generous gifts from Dr. Charles S. Abrams. Myc-PI3Kα was a generous gift from Dr. Michael J. Waterfield.

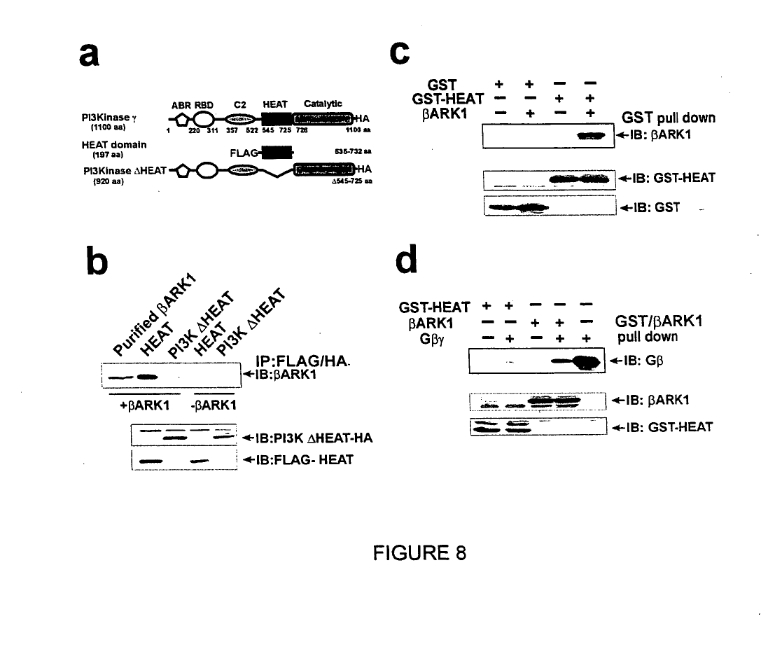

HEAT and PI3KΔHEAT mutants of PI3K were prepared by polymerase chain reaction (PCR) amplification using the full-length p110γ cDNA as template (FIG. 8a). The HEAT domain was amplified using Pfu platinum turbo Taq high fidelity enzyme (STRATAGENE) with the 5′ primer (5′-TCTCGAGGATCCGCCGCCATGGACTACA AGGACGACGATGATAAGCACCCGATAGCCCTGCCT-3′) containing XhoI and BamHI sites for subcloning, followed by Kozak consensus sequence and a FLAG epitope tag and the 3′ primer (5′-GTCGACCTAGTCGTGCAGCATGGC-3′) containing consensus stop codon with a SalI site for subcloning. The PCR product was subcloned in zero-blunt TOPO vector (Invitrogen) and sequence verified for authenticity. Following digestion with the restriction enzymes BamHI and SalI, the HEAT domain-fragment was subcloned into the following expression plasmids: the pRK5 mammalian expression vector, the pGEX-4T bacterial expression vector to generate GST-fusion proteins and the enhanced green fluorescent protein vector pEGFP-C1.

PI3KΔHEAT was constructed using 2 PCR reactions that selectively amplified regions of PI3K upstream and downstream from the HEAT domain (FIG. 8a). The region of PI3K upstream from HEAT was amplified using the forward primer (5′-tgcggatccgccaccatggagctggagaactataaacag-3′) containing a BamHI site and Kozak consensus sequence, and the reverse primer (5′-TTCTGCTCGACCGCGGTCCCCTTCCGG-3′) containing a SacI site. The region of PI3K downstream of the HEAT domain was amplified using the forward primer (5′-CTGAGGGGCCGCGGCACAGCCATG-3′) containing a SacI site and the reverse primer (5′-ACCCGGGATCCTTAAGCGTAGTCTGGTACGT-3′) containing a BamHI site and a consensus stop codon. The upstream and downstream regions of PI3K were separately subcloned in the zero-blunt TOPO vector and sequence was verified by dideoxy sequencing. Following digestion with the restriction enzymes BamHI/SacI, a three fragment ligation was carried with the mammalian expression vector pRK5, to generate the plasmid containing the PI3KΔHEAT cDNA (FIG. 8a). The catalytic activity of PI3KΔHEAT was indistinguishable from wild type PI3K. The plasmids containing cDNAs encoding βARK1, FLAG-β₂AR, HA-β₂AR and p110γ have been described previously.

Cytosolic and Membrane Fractionation—Cell monolayers were scraped in 1 ml of buffer containing 25 mM Tris-HCl (pH 7.5), 5 mM EDTA, 5 mM EGTA, 1 mM phenylmethylsulfonyl fluoride, 2 μg/ml each of leupeptin and aprotinin and disrupted further by using Dounce homogenizer. Intact cells and nuclei were removed by-centrifugation at 1,000×g for 5 min. The collected supernatant was further subject to a centrifugation at 38,000×g for 25 min. The pellet was resuspended in lysis buffer (1% Nonidet P-40, 10% glycerol, 137 mM NaCl, 20 mM Tris-Cl (ph 7.4), 1 mM phenylmethylsulfonyl fluoride, 20 mM NaF, 1 mM sodium pyrophosphate, 1 mM sodium orthovanadate and 2 μg/ml each of aprotinin and leupeptin) and used as membrane fraction and the supernatant was diluted in lysis buffer and used as the cytosolic fraction. Heart samples also underwent through similar procedures to obtain membrane and cytosolic fractions. Purity of the membrane fraction preparation was confirmed by measuring enzyme activity of the membrane marker enzyme K⁺-stimulated p-nitrophenylphosphatase (membrane fraction: 9.4 μmol/mg protein per min, cytosol: 2.6 μmol/mg of protein/min).

Lipid Kinase Assays—Cells were lysed in lysis buffer and 2 mg cytosolic extract was used for immunoprecipitation with either the C5/1 monoclonal antibody directed against βARK1, the anti-FLAG M2 monoclonal antibody (Sigma) or antibodies to p110γ, or p110β (Santa Cruz Biotechnology) and protein G-agarose. Sedimented beads were then washed once with 1 ml of lysis buffer, thrice with 1 ml of phosphate-buffered saline (1× phosphate-buffered saline, 1% NP-40 and 100 μM sodium orthovanadate), thrice with 1 ml of Tris/LiCl (100 mM Tris-HCl (pH 7.4), 5 mM LiCl and 100 μM sodium orthovanadate) and twice with TNE (10 mM Tris Cl (pH 7.4), 150 mM NaCl, 5 mM EDTA and 100 μM sodium orthovanadate). Samples were then resuspended in 50 ml of TNE and 10 μl of 100 mM MgCl₂ was added to each sample. The substrate, phosphatidylinositol (PI, Avanti), was prepared by drying 50 μl of the 10 mg/ml stock (PI in chloroform) in an eppendorf tube with a stream of air and adding 250 μl of TE (10 mM Tris Cl (pH 7.4) and 1 mM EDTA). The PI was then suspended by sonication in an ice bath for 3 minutes. 10 p, of the PI solution and 11 μl of ATP solution (440 μM cold ATP and 1 μCi/μl of [γ-32P]ATP) were added to each sample. Samples were incubated at 22° C. for 10 min with continuous agitation. Reactions were terminated by adding 20 μl of 6N HCl. 160 p, of a 1:1 chloroform:methanol solution was added to each sample and samples were centrifuged at 14,000 RPM for 10 minutes, thus separating each sample into 3 phases. 40 μl of the lower (chloroform/lipid) phase was spotted onto 200-μm silica-coated flixi-TLC plates (Selecto-flexible, Fischer Scientific) precoated with 1% potassium oxalate and resolved chromatographically in 2 N glacial acetic acid:1-propanol (1:1.87). Autoradiography of dried plates was used to detect signals, and PIP was quantified by phosphorimaging. Ptdlns(4)P (Sigma) was used as a standard. Values are expressed as fold increase/decrease relative to wild-type/control.

Immunoblotting and Detection—Immunoblotting and detection of PI3K, βARK1, HA-PI3Kγ, HA-ΔPI3Kγ, Myc-PI3Kα, FLAG-HEAT, PI3KΔHEAT-HA, FLAG-β₂AR, HA-β₂AR, β-adaptin and clathrin was carried out as previously described. Immunoprecipitating antibodies were added to 500 μg of cell lysate in lysis buffer followed by the addition of 35 μl of 1:1 Protein A- or G-agarose. Samples were rocked overnight at 4° C. then centrifuged at 12,000×g for 5 min. Immunoprecipitates were washed twice with lysis buffer, twice with 1×PBS and resuspended in 1×SDS-gel loading buffer. Proteins were resolved by SDS-polyacrylamide gel electrophoresis and blotted on to PVDF membrane (Bio-Rad). Blots were incubated with antibodies recognizing PI3K, Myc (Santa-Cruz), β-adaptin, clathrin heavy chain (BD Transduction Laboratories), HA (Roche Molecular Biochemicals) and FLAG (Sigma) at 1:2000 dilution and the βARK1 monoclonal antibody at 1:10,000 dilution. Blots were subsequently incubated with appropriate secondary antibody (1:2000 dilution) conjugated to horseradish peroxidase (Amersham Pharmacia Biotech) and detection was carried out using enhanced chemiluminescence.

Determination of β₂AR Sequestration in HEK-293 Cells by ¹²⁵I-Cyanopindolol (CYP) Binding—β₂AR sequestration was performed as previously described. Briefly, HEK-293 cells were plated at a density of 2.5×10⁶ cells/dish and transfected the following day with plasmids containing either the β₂AR (150-250 ng), β₂AR (250 ng) and HEAT domain (4 μg), PI3Kγ (4.0 μg) or ΔPI3Kγ (4.0 μg) cDNAs. Twelve hours after transfection cells were split into six well Falcon plates at a density of 750,000 cells/well. The following day the media was replaced with MEM containing 1 μM isoproterenol and 100 μM ascorbate for 0 to 30 min. In separate experiments, cells transfected with β₂AR were treated with the PI3K inhibitors wortmannin (100 nM) or LY294002 (100 μM) for 15 min before isoproterenol stimulation. To determine the amount of internalized receptor, 100 μl aliquots of whole cells were added to 150 μl of binding buffer (75 mM Tris-HCl, 10 mM MgCl₂, 5 mM EDTA (pH 7.5)). Total binding was determined in the presence of 175 pM ¹²⁵I-CYP alone, the number of internalized receptors were determined by using 175 pM ¹²⁵I-CYP plus 100 nM CGP12177, and non-specific binding was determined using 175 pM ¹²⁵I-CYP plus 1 μM propranolol. Sequestration was calculated as the ratio of (specific receptor binding of ¹²⁵I-CYP in the presence of CGP12177)/(specific receptor binding of ¹²⁵I-CYP in the absence of CGP12177).

Confocal Microscopy—Confocal microscopy was performed as previously described. In brief, HEK-293 cells were transfected with plasmids containing the β₂AR (2 μg) and β-arrestin-GFP (2 μg), the β₂AR and β-arrestin-GFP along with either PI3Kγ (2.5 μg) or ΔPI3Kγ (2.5 μg), the β₂AR-YFP (2 μg), or β₂AR-YFP (2 μg) and HEAT domain (4 μg). Cells were split into 35 mm² dishes with glass bottoms for observation using a Zeiss LSM-510 confocal microscope. The cells were treated with isoproterenol (1 μM) for the indicated times (0 to 5 min) and the fluorescence images were exported as Tiff files by the LSM software to Adobe Photoshop. For analyses of the HEAT domain, HEK 293 cells were transfected with the plasmids containing cDNAs encoding either the β₂AR-YFP (2 μg) or β₂AR-YFP (2 μg) and HEAT domain (4 μg). Cells were plated onto glass bottom dishes for observation in the confocal microscope. Live cells were treated with isoproterenol (10 μM) and images were collected sequentially over a time course of 0 to 10 min. For dual staining of β₂AR-HA or FLAG-HEAT, cells were fixed in 4% paraformaldehyde in phosphate-buffered saline for 30 min following 10 min of 10 μM isoproterenol stimulation. Cells were permeablized with 0.1% Triton X-100 in phosphate-buffered saline for 20 min, incubated in 1% BSA in phosphate-buffered saline for 1 hr. Cells were washed with 1× phosphate-buffered saline and incubated with anti-HA or anti-FLAG monoclonal antibody (1:250) with 1% BSA in 1× phosphate-buffered saline for 1 hr. Cells were washed and incubated with goat anti-mouse IgG conjugated with Texas Red (1:500) (Molecular Probes) for 1 hr. Samples were visualized using single sequential line excitation filters at 488 and 568 nm and emission filter sets at 505-550 nm for GFP detection and 585 for Texas Red detection.

β₂AR Phosphorylation—β₂AR phosphorylation was performed in intact cells as previously described. Cells were transfected with plasmids containing the FLAG β₂AR (2 μg) and vector DNA (2.5 μg), the FLAG β₂AR (2 μg) and PI3Kγ (2.5 μg), or the β₂AR (2 μg) and ΔPI3Kγ (2.5 μg). 24 hr after transfection, cells were washed and metabolically labeled for 1 hr with medium containing 100 μCi of ³²P/ml. Following stimulation with 10 μM isoproterenol for 5 min, incubations were terminated by adding 2 ml of ice-cold phosphate-buffered saline/well, and then cells were solubilized with the addition of 0.75 ml/well of radioimmune precipitation buffer. Following centrifugation at 38000×g for 20 min at 4° C., the supernatants were processed for immunoprecipitation of the FLAG-β₂AR as described above. Phosphorylated receptors were resolved by 10% SDS-polyacrylamide gel electrophoresis and dried gels subjected to autoradiography.

In vivo pressure overload hypertrophy and isoproterenol infusion—Four month old adult C57BL/6 wild type mice of either sex were used for this study. Microsurgical procedures and hemodynamic evaluation of pressure overload hypertrophy induced through transverse aortic constriction (T) was performed as previously described. After 7 days of aortic constriction, mice were anesthetized and both carotid arteries were cannulated to measure the trans-stenotic pressure gradient. Hearts were then rapidly excised, individual chambers were separated, weighed and frozen in liquid N₂ for later biochemical analysis. In separate experiments, adult wild type mice underwent intravenous infusion of 10 μM isoproterenol for 3 min at 50 μl/min. Hearts were removed and flash frozen in liquid N₂ for later biochemical analysis. The animals in this study were handled according to approved protocols by the animal welfare regulations of Duke University Medical Center.

GST Fusion Protein Expression and Pull-down Experiments—Plasmid DNAs were transformed in Escherichia coli BL21 cells. Overnight cultures were grown in LB medium supplemented with ampicillin (100 μg/ml), diluted to an A₆₀₀ of 0.2 in the same medium and grown for another 1 hr at 37° C. Cultured cells were then induced with 0.1 mM isopropyl-1-thio-β-D-galactopyranoside for 2 hr. Cell were then pelleted, washed once with 1× phosphate-buffered saline and resuspended in 1× phosphate-buffered saline containing 1 mM phenylmethylsulfonyl fluoride, 2 mg/ml lysozyme and incubated for 15 min on ice. Cells were lysed by adding Triton X-100 1%. Solublized cells were incubated with DNase (300 units) for 15 min on ice and centrifuged at 13,000 rpm for 10 min.

Glutathione-Sepharose beads were added to the supernatant and gently agitated at 4° C. for 2 hr. Beads were washed three times with ice cold 1×PBS containing 1% Triton X-100 followed by three washes with cold PBS without detergent. Protein concentration was determined using a DC protein assay kit (Bio Rad), and the integrity of the fusion protein was analyzed by SDS-polyacrylamide gel electrophoresis and Coomassie staining.

GST fusion proteins (1-1.5 μg) on beads were incubated in 0.5 ml of binding buffer (10 mM Tris-HCl (pH 7.4), 5 mM EDTA, 0.2% Triton X-100) for 2 hr at 25° C. together with purified βARK1 protein (5 μg). The beads were spun and washed three times with binding buffer followed by three washes with binding buffer without detergent. The beads were resuspended in SDS gel loading buffer and resolved by gel electrophoresis, immunblotting and detection was carried out described later.

Immobilized βARK1 protein was prepared by incubating βARK1 monoclonal antibodies with protein G agarose beads for 1 hr at 4° C., followed by the addition of purified βARK1 protein. Subsequently, 10 μg of purified Gβγ was added to either βARK1 immobilized beads (5 pg), or the GST-HEAT fusion protein beads (5 μg), and gently rocked for 45 min at room temperature. Beads were spun down, washed in binding buffer X2 and resolved by gel electrophoresis. The presence of Gβγ was detected by immunoblotting with an antibody directed against the Gβ subunit. Purified Gβγ and βARK1 were gifts from Dr. Robert Lefkowitz.

Treatment of cells with phospholipids—Modification of a previously described method was used to vary the concentration of D-3 phospholipids in living cells. Briefly, one of the synthetic phospholipids DiC16 Ptdlns-3-P, DiC16 Ptdlns (4,5) P₂, or DiCPtdlns (3,4,5) P₃ (AVANTI), was mixed with phosphatidylcholine and phosphoinositol (Sigma) at a 1:100:100 ratio and dried under N₂. Phospholipids were then re-suspended in 10 mM HEPES (pH 7.4) containing 1 mM EDTA and sonicated. Cells were treated with Saponin (0.04 mg/ml) in serum free medium along with the vesicles containing the phospholipids at given concentration of Ptdlns-3-P or Ptdlns (4,5) P₂ or Ptdlns (3,4,5) P₃ for 10 min at 25° C. Cells were then treated with isoproterenol (10 μM) for 5 min at 37° C., then lysed for immunoprecipitation experiments with a buffer containing 0.8% Triton X-100, 20 mM Tris-HCl (pH 7.4), 300 mM NaCl, 1 mM EDTA, 20% glycerol, 0.1 mM PMSF, 10 μg/ml leupeptin and aprotinin.

Statistical Analysis—Data is expressed as mean±SEM. Statistical comparisons was performed using an unpaired Student's t test and ANOVA where appropriate. Results for the β₂AR sequestration by CYP binding was analyzed using Graphpad Prism.

Transgene Construction—The HotSHOT protocol was utilized to genotype the mice. Approximately 0.2 cm of tail was clipped from each mouse and added to 75 μl of alkaline lysis buffer (25 mM NaOH and 0.2 mM EDTA). Samples were boiled at 95° C. for 10 min then cooled to 4° C. 75 μl of neutralizing reagent (40 mM Tris-HCl) was then added to each sample. 1 μl of this solution was added to 22 μl of ddH₂O along with 1 μl (15 μM) of each of 2 transgene-specific primers (Sigma-Genosys) and a PCR bead containing the Taq polymerase, MgCl₂ and dNTPs (Amersham Pharmacia Biotech). Samples were then placed in a Peltier Thermal Cycler (PTC-200—MJ Research) and cycled 30 times at [94° C. for 30 sec, 65° C. for 30 sec and 72° C. for 45 sec]. 10 μl of each sample was loaded onto a 2% agarose:TAE gel containing ethidium bromide. Bands were detected with a GelDoc 2000 (Bio-Rad).

Transthoracic Echocardiography—Transthoracic 2D guided M-mode echocardiography was performed on conscious, unanesthetized mice at 3-4 months of age using an HDI 5000 echocardiograph (ATL, Bothell, Wash.) as described previously.

Miniosmotic Pump Implantation—Miniosmotic pumps were inserted. Transgenics with 180-fold expression of the mutant protein and their wild-type littermates were anesthetized with a mixture of ketamine (10 mg/kg) and xylazine (0.5 mg/kg), and a small incision was made in the skin between the scapulae. A small pocket was created by spreading apart the subcutaneous connective tissue. Isoproterenol was dissolved in 0.002% ascorbic acid, and pumps were filled to deliver at a rate of 3 mg/kg/day over a period of 7 days. After insertion of the miniosmotic pump (model 2002; Alzet), the skin incision was closed with a 4.0 catgut suture. As controls, pumps that delivered vehicle (0.002% ascorbic acid) were implanted in mice. At the end of 7 days, hemodynamic evaluation was performed as described below.

Hemodynamic Evaluation in Intact Anesthetized Mice—Hemodynamic evaluation in intact mice was performed as described previously. Mice were anesthetized with a mixture of ketamine (100 mg/kg) and xylazine (2.5 mg/kg) and, after endotracheal intubation, were connected to a rodent ventilator. After bilateral vagotomy, the left carotid artery was cannulated with a flame-stretched PE-50 catheter connected to a modified P-50 Statham transducer. A 1.4 French (0.46 mm) high-fidelity micromanometar catheter (Millar Instruments, Houston, Tex.) was inserted into the right carotid and advanced retrograde into the left ventricle. Hemodynamic measurements were recorded at baseline and 45 seconds after injection of incremental doses of isoproterenol (50, 500 and 1000 pg). Following the 1000 pg dose, the hearts were explanted, rinsed in cold phosphate-buffered saline, and blotted dry. After weighing, isolated hearts were frozen in liquid nitrogen and stored at −80° C. until needed for biochemical studies.

Immunoblotting and Detection for transgenic mice experiments—Immunoblots were performed as described previously. Cytosolic extracts were prepared as above and 70-140 μg of pure cytosolic extract was combined with 10 μl of 1× Laemmli loading buffer and boiled at 100° C. for 5 min, then placed on ice. The samples were electrophoresed on a 8-10% SDS-PAGE gel (Proto-Gel—National Diagnostics). Gels were then placed in a 4° C. transfer buffer (39 mM glycine, 48 mM Tris base and 20% methanol) tank and the proteins were transferred to a PVDF membrane (Bio-Rad) soaked in methanol. Membranes were then washed in TBS-T (25 mM Tris base (pH 7.6), 1.37 M NaCl and 0.1% tween-20) and blocked for 1 hour with blocking buffer (5% nonfat dry milk in TBS-t). The membrane was then incubated overnight in blocking buffer containing the appropriate 1° antibody (p110γ, HA, PKB, GSK3β, p70-S6K, JNK, ERK, p38, p38β, βARK1 (Santa Cruz Biotechnologies) phospho-PKB (Ser-473), phospho-GSK3β (Ser-9) and phospho-p70-S6K (Thr-389 & 421/424) (Cell Signalling)) then washed thoroughly with TBS-t. Finally, the membrane was incubated in blocking buffer containing a 1:2000 dilution of the appropriate 2° (anti-rabbit, goat (p38β) or mouse (HA)) antibody (Amersham) conjugated with horseradish peroxidase. The membranes were then incubated in ECL (Amersham) and the chemiluminescent bands were detected by autoradiography. Bands were quantified with a densitometer (Bio-Rad) and values are expressed as fold increase/decrease relative to wild-type.

Ligand Binding Assays—Membrane fractions were prepared from left ventricles via homogenization in 2 ml of lysis buffer (Buffer-A—25 mM Tris base (pH 7.5), 5 mM EDTA, 5 mM EGTA, 10 μg/ml Leupeptin, 20 μg/ml Aprotinin and 1 mM PMSF). Samples were centrifuged at 1000×g for 10 min and the supernatant placed in a new tube and centrifuged at 48,000×g for 30 min. The pellets were resuspended in 1 ml of β-binding buffer (75 mM Tris-HCl, pH 7.4, 12.5 mM MgCl₂, and 2 mM EDTA), centrifuged at 18,000 rpm for 30 min and again resuspended in 500 μl of β-binding buffer. Binding assays were performed on 25 μg of membrane protein using saturating amounts of ¹²⁵I-CYP (300 pM), a β-AR-specific ligand. Nonspecific binding was determined in the presence of 20 μM alprenolol. Reactions were conducted in either 250 or 500 μl of binding buffer at 37° C. for 1 hr and then terminated by vacuum filtration through glass-fiber filters. The β-AR-bound CYP trapped in the filter was then quantified with a gamma counter. All assays were performed in triplicate, and receptor density (fmol) is reported as picomoles of receptor per milligram of membrane protein.

Adenylyl Cyclase Activity—Assays were performed as described previously and membrane fractions were prepared as above. 20 μl of sample was added to tubes containing 10 μl of one of three solutions (in triplicates): 1 mM ascorbic acid, 50 mM sodium fluoride or 0.5 mM isoproterenol. 20 μl of cyclase mix (2.7 mM phosphoenol pyruvate (PEP), 53 μM GTP, 0.1 mM cAMP, 0.12 mM ATP, 1 IU myokinase, 0.2 IU pyruvate kinase and 50 μCi/ml [α-³²P]ATP) was added to each tube and incubated for 15 min at 37° C. Reactions were quenched by adding 1 ml of stop solution (200 mg/L ATP, 100 mg/L cAMP and 30 μl/L ³H-cAMP). Samples were poured into dowex columns (Bio-Rad) and cAMP was eluted into alumina columns (ICN) with 4.5 ml of H₂O. cAMP was eluted out of the alumina columns and into scintillation vials with 4 ml of 0.1 M imidazole (pH 7.65). 5 ml of Ecoscint (National Diagnostics) was then added to each vial. The amount of cAMP generated was quantitated with a liquid scintillation counter (MINAXIβ-4000).

### Example 1

### βARK1 and PI3K Form a Complex in the Cytoplasm

βARK1 and PI3K interacted to form a cytosolic complex, as illustrated in FIG. 3. NIH-3T3 cells, which has a relatively low level of endogenous PI3K activity, were transfected with βARK1, HA-PI3Kγ cDNAs, or control vector DNA. βARK1 was immunoprecipitated from extracts using a monoclonal antibody directed against the βARK1 catalytic domain. As shown in FIG. 3A-C, PI3K activity and protein was found associated with βARK1. Furthermore, the association was independent of PI3K activity since co-transfection with a catalytically inactive PI3K mutant (HA-ΔPI3Kγ, deletion in the ATP binding site) did not prevent the association of the PI3K mutant with βARK1 (FIG. 3C). A, 48 hours after transfection 500 μg of cytosolic extract was immunoprecipitated using βARK1 monoclonal antibody directed against-its catalytic domain and the associated lipid kinase activity was measured. Shown is a representative autoradiograph of a TLC plate where PIP and phosphatidylinositol bisphosphate (PIP2) are visualized. Ori, origin of resolution. B. βARK1 associated PI3K activity quantified by phosphorimaging of the TLC plates from four independent experiments. Results are expressed as fold over control. *, p<0.05 vs. control. C, Immunoprecipitations (IP) were performed from cytosolic extracts with an anti-HA or anti-βARK1 monoclonal antibody and immunoblotted (IB) with a βARK1 monoclonal (upper panel) or anti-HA monoclonal antibody (lower panel). βARK1 and HA-PI3Kγ transfected cells were used as positive controls (last lane).

Furthermore, the association of PI3K with βARK1 occurred with either PI3K isoform (FIG. 3D) and the associated PI3K activity was wortmannin sensitive (FIG. 3E). D, 500 μg of cytosolic extract was immunoprecipitated with a βARK1 monoclonal antibody and the associated lipid kinase activity was measured. Shown is a representative autoradiograph where PIP is visualized. E, 500 μg of cytosolic extract was immunoprecipitated with a βARK1 monoclonal antibody and the associated PI3K activity was measured from cells with and without treatment with 100 nM wortmannin (Wort) for 15 minutes prior to lysis. Lysates from cell extracts prior to immunoprecipitation were immunoblotted for βARK1, PI3Kγ and ΔPI3K to determine levels of expression.

### Example 2

### Agonist-Dependent ΔARK1 Mediated Translocation of PI3Kγ

The present inventors determined that βARK1 and PI3Kγ form a complex in the cytosol and translocate to the membrane on agonist stimulation in a Gβγ dependent manner, as illustrated in FIG. 4.

Experiments were performed in N1H-3T3 cells co-transfected with βARK1 and HA-PI3Kγ cDNAs, then the endogenous βAR receptors were stimulated with 10 μM isoproterenol for 2 min. Cytosolic and membrane fractions were prepared and analyzed for βARK1 associated PI3K activity, by immunoprecipitating βARK1 from 250 μg of protein from the membrane and cytosolic fraction and assaying for associated PI3K activity. As illustrated by a representative autoradiograph of a TLC plate where PIP is visualized, little change in the βARK1 associated PI3K activity was noted in the cytosolic fraction after isoproterenol stimulation (FIG. 4A). In contrast, the membrane fraction showed a significant increase in the βARK1 associated PI3K activity (FIGS. 4A and D) at 2 min following agonist stimulation. FIG. 4D illustrates βARK1 associated PI3K activity quantified by phosphorimaging the TLC plates from four independent experiments. Results are expressed as fold over basal (no isoproterenol treatment). *, p<0.05 vs. membrane fraction without ISO treatment.

The βARK1 mediated recruitment of PI3Kγ to the membrane was analyzed by immunoprecipitating βARK1 from both fractions and blotting for HA-PI3K. As shown in FIG. 4B, treatment with isoproterenol resulted in a greater level of βARK1 associated PI3Kγ protein in the membrane; cells transfected with HA-PI3Kγ cDNA was used as a positive control. MEM, membrane fraction, CYT, cytosolic fraction. Taken together these data show that βARK1 and PI3Kγ interact to form a complex in the cytosol, and βARK1 recruits PI3Kγ to the membrane in an agonist-dependent manner.

### Example 3

### Agonist-Dependent Translocation of βARK1 Associated PI3K Activity is Gβγ Dependent

The present inventors determined that Gβγ subunits were required for the translocation of the βARK1/PI3Kγ complex to the membrane by overexpressing the C-terminal portion of βARK1, the βARKct, which is known to inhibit the ability of βARK1 to bind Gβγ. Cells were co-transfected with the βARK1 and PI3Kγ plasmids, along with or without the βARKct cDNA (the carboxyl terminus of βARK1 that attenuates the Gβγ dependent signaling). Robust PI3K activity was found associated with immunoprecipitated βARK1 in the absence of βARKct (FIG. 4C), determined by immunoprecipitating βARK1 with a βARK1 monoclonal antibody and assaying for associated PI3K activity following isoproterenol stimulation (2 min). In contrast, the agonist-dependent translocation of βARK1 associated PI3K activity was abolished in the presence of the βARKct (FIGS. 4C and D). These data demonstrate that the sequestration of Gβγ by βARKct can interrupt the process of βARK1-mediated translocation of PI3Kγ to the membrane (FIG. 4D). Interestingly, in the presence of P3ARKct there was also loss of βARK1 associated PI3K activity in the cytosolic fraction (FIGS. 4C and D).

The present inventors determined that βARKct, which contains the same PH domain as βARK1, competitively inhibits the βARK1/PI3K interaction. They transfected N1H-3T3 cells with cDNAs of P3ARK1, P3ARKct, and either PI3Kγ or PI3Kα, and immunoprecipitated with antibodies directed against either HA-PI3Kγ or myc-PI3Kα. As shown in FIG. 4E, both βARK1 and βARKct were found to interact with either of the PI3K isoforms, as determined using a polyclonal antibody against the C-terminus of βARK1 that recognizes both full length βARK1 and the βARKct. To exclude the possibility that overexpression of the βARKct altered the level of expression of either βARK1 or PI3Kγ, lysates prepared from the co-transfected cells were immunoblotted for βARK1, βARKct and PI3Kγ. No significant difference in the level of expression levels for either βARK1 or PI3Kγ was seen in presence of the βARKct. Endogenous βAR density of N1H-3T3 cells was determined by ¹²⁵I cyanopindolol binding study and found to contain 20.7+3.2 fmol/mg whole cell protein, which is sufficient to activate and translocate βARK1 following agonist stimulation. These data further point to the C-terminal PH domain of βARK1 as the interacting domain since it is common within both βARKct and βARK1. Overexpression of the βARKct acts to inhibit the recruitment of βARK1 to the membrane by sequestering Gβγ subunits and also directly interrupts the βARK1-PI3Kγ interaction.

### Example 4

### βARK1 Translocates PI3K to βAR

The present inventors determined that the ability of βARK1 to translocate PI3K to the cell membrane is a mechanism for co-localization of PI3K with P3ARs. They used HEK-293 cells transfected with either FLAG epitope tagged β₁AR or β₂AR plasmids and monitored the association of PI3K to the receptor after agonist stimulation. HEK-293 cells are known to contain adequate levels of βARK1 to support agonist-induced receptor phosphorylation. Transfected cells were split into separate dishes and stimulated with 10 μM isoproterenol from 0 to 10 minutes. The FLAG epitope was immunoprecipitated from 500 ug of protein from the cytosolic extracts and PI3K activity measured. As shown in FIG. 5A-D, FLAG β₁AR and FLAG β₂AR associated PI3K activity was observed by 2 minutes following agonist stimulation with gradual decline by 10 minutes.

Moreover, the PI3K activity associated with the FLAG β₁AR and β₂AR was wortmannin sensitive (FIG. 5E). HEK-293 cells were transfected with FLAG β₁AR (upper panel) and FLAG β₂AR (lower panel). A set of transfected cells were treated with 100 nM wortmannin 15 min prior agonist stimulation. Both wortmannin treated and untreated cell were then stimulated with 10 μM isoproterenol for the indicated times. Cell extracts were used to immunoprecipitate β₁AR and β₂AR using the anti-FLAG antibody and PI3K activity was measured in the immunoprecipitates. Shown are the autoradiograph of TLC plates where PIP was visualized. Agonist dependent association of endogenous PI3Kγ protein with the transfected FLAG β₂AR at 2-10 min after isoproterenol treatment was also detected.

### Example 5

### PI3K Activity is Required for the Sequestration of the Receptor

The present inventors determined that PI3K activity was required for GPCR sequestion. In the presence of PI3K inhibitors, wortmannin and LY294002, agonist-dependent (1 μM isoproterenol) sequestration was studied in HEK-293 cells transfected with the FLAG β₂AR plasmid. As shown in FIG. 6A, a significant attenuation in the rate of β₂AR sequestration was observed in wortmannin treated cells for up to 20-30 min, as shown by ¹²⁵I-Cyanopindolol binding over a time course of 0 to 30 minutes in untreated (▪) and wortmannin (100 nM) treated (□) cells. Receptor sequestration at each time point was normalized to the value of internalized β₂AR at 30 min in the absence of wortmannin. The maximal absolute level of sequestration for the β₂AR in the untreated cells at 30 min was 22.3±3.9%, n=5. Receptor expression (fmol/mg whole cell-protein) was 378±123, *, p<0.05 vs. untreated. Inset: upper panel, inhibition of endogenous lipid kinase activity with wortmannin. β₂AR and +, represents β₂AR transfected wortmannin treated cells; β₂AR and − represents β₂AR transfected untreated cells. The first lane represents endogenous PI3K activity in the untransfected cells. Lower panel, the level of endogenous PI3Kγ expression. Similarly, a 50% reduction in sequestration was also observed with LY294002 treated cells. The data clearly show that in transfected HEK293 cells, the presence of agonist promotes the association of wortmannin sensitive PI3Kγ activity with both β1 and β₂ adrenergic receptors.

Demonstrating that PI3K is required for the process of βAR sequestration, a time course of agonist-dependent sequestration was studied in HEK-293 cells transfected either with the FLAG β₂AR (▪), FLAG β₂AR and PI3Kγ (□), or FLAG β₂AR and ΔPI3Kγ (◯) plasmids. Similar to the pattern of inhibition by wortmannin, ΔPI3Kγ transfected cells showed a significant attenuation in the rate of receptor sequestration (FIG. 6B). These data demonstrate a role for PI3K in the process of βAR internalization possibly due to the local production of Ptdlns (3,4,5) P₃. In FIG. 6B, receptor expression (fmol/mg whole cell-protein) was: β₂AR+vector, 316±75; β₂AR+PI3Kγ, 367±81; β₂AR+API3Kγ, 267±157. N=5, *, p<0.05 vs. β₂AR. (Inset upper panel), Immunoblot showing the levels of expression of HA-PI3Kγ, HA-API3Kγ and PI3Kγ in the transfected cells. Cells were transfected with β₂AR (−), β₂AR and wild type PI3Kγ (WT) or β₂AR and ΔPI3Kγ (Δ) cDNAs. (Inset lower panel), lipid kinase activity in the HEK 293 cells transfected with the PI3Kγ (WT) and API3Kγ (Δ) cDNAs. The receptor sequestration at each time point has been normalized to the value of internalized β₂AR at 30 min. The maximal absolute level for the β₂AR in the untreated cells at 30 min was 25.0±2.1%, n=5. Treatment with PI3K inhibitors as shown above, or overexpression of catalytically inactive PI3K, attenuated β₂AR sequestration.

Alteration of receptor endocytosis with overexpression of the ΔPI3Kγ mutant could result if ΔPI3Kγ inhibited either recruitment of β-arrestin to the phosphorylated receptor or directly inhibited receptor phosphorylation. To exclude these possibilities cells were co-transfected with plasmids containing FLAG β₂AR, β-arrestin2-GFP and either PI3Kγ or ΔPI3Kγ cDNAs, and were monitored for the recruitment of β-arrestin2-GFP using confocal microscopy. In cells transfected with API3Kγ there was prompt recruitment of β-arrestin2-GFP to the receptor after exposure to agonist (FIG. 6C). Marked redistribution of β-arrestin2-GFP to the membrane occurred within 2.5 minutes. Arrows on the 5 min panel highlight regions of translocated β-arrestin2-GFP. The upper panel is an immunoblot for HA from extracts of the same cells showing equal levels of HA-PI3Kγ and HA-ΔPI3Kγ expression.

To directly test the effect of API3Kγ expression on receptor phosphorylation, ³²P; metabolic labeling was performed in HEK-293 cells transfected with plasmids containing FLAG β₂AR and either the PI3Kγ or ΔPI3Kγ cDNAs. As shown in FIG. 6D, the level of agonist-induced receptor phosphorylation was not affected by the expression of ΔPI3Kγ. Thus, the effect of overexpression of ΔPI3Kγ on β₂AR sequestration is not related to processes that are involved with β₂AR phosphorylation or β-arrestin2 recruitment. In FIG. 6D, Agonist (ISO, 5 min) promoted β₂AR phosphorylation in transfected cells after ³²Pᵢ metabolic labeling for 1 hour prior to stimulation (upper panel). Lower panel, immunoblot for HA showing equal levels of expression of HA-PI3Kγ and HA-ΔPIβKγ. + represents HA-PI3Kγ control.

These studies thus demonstrate that the agonist-dependent recruitment of PI3K to the membrane is an important step in the process of receptor sequestration and links class I PI3Ks to GPCR signaling and endocytosis.

### Example 6

### βARK1 and PI3K Form a Complex in the Heart

The present inventors determined that PI3K and βARK1 interact in the heart; normal βAR function is critical for maintenance of cardiac function, particularly during periods of increased workload. The monoclonal βARK1 antibody was used to immunoprecipitate βARK1 from 4 mg of myocardial extracts prepared from normal mouse hearts. The immunoprecipitate was tested for the presence of associated PI3K protein by immunoblotting. As a positive control, the PI3K polyclonal antibody was used to immunoprecipitate total PI3K from 2 mg of separate extracts prepared from the same heart. As shown in FIG. 7A, PI3K was co-immunoprecipitated along with βARK1 from the myocardial extract.

The association of βARK1 with PI3K in the heart would also resulted in the βARK1 mediated translocation of PI3K to the membrane; anesthetized mice were stimulated with isoproterenol (10 μM) by infusion through a cannulated jugular vein for 3 min. Membrane and cytosolic fractions were prepared from the treated hearts and PI3K activity measured following immunoprecipitation with the βARK1 monoclonal antibody. As show in FIG. 7B, a significant increase in βARK1 associated PI3K activity was found in the membrane fraction following isoproterenol treatment (fold induction over untreated 5.60+1.10, p<0.05, n=3). No difference in βARK1 associated PI3K activity was found in the cytosolic fraction following isoproterenol treatment (fold induction over untreated 1.19+0.47, n=3).

The present inventors determined that the interaction of βARK1 with PI3K was also enhanced in the hypertrophied heart. As shown in FIGS. 7C and D, greater wortmannin sensitive PI3K activity and protein was found complexed with βARK1 in hypertrophied hearts compared to sham treated hearts. In FIG. 7C, βARK1 associated PI3K activity was measured in the myocardial extracts from the sham (S) and transverse aortic constricted (T) hearts. 4 mg of the myocardial extracts was used for immunoprecipitation with βARK1 monoclonal antibody and then assayed for the PI3K activity (fold induction in-T over S 2.1+0.13, p<0.05, n=3). −Wort and +Wort, reactions performed in the absence or presence of wortmannin. In FIG. 7D, Myocardial extracts from sham and T hearts were used to immunoprecipitate βARK1 and PI3Kγ with a monoclonal antibody directed against βARK1 (4 mg of extract) and a polyclonal antibody for PI3Kγ (2 mg of extract) respectively as a positive control. The protein bands were visualized using ECL chemiluminescence. T, Transverse aortic constriction.

The present inventors demonstrated an important role of PI3Kγ in the process of βAR internalization, possibly due to the local production of Ptdlns (3,4,5) P₃. Overexpression of API3K neither inhibited the β-arrestin2-GFP recruitment to the receptor, nor βARK1 mediated β₂AR phosphorylation. In the unstimulated heart, βARK1 and PI3K formed a complex in myocardial extracts. Importantly following isoproterenol stimulation there was increased βARK1 associated PI3K activity in myocardial membranes corroborating the cell culture studies. In the hypertrophied heart there is an increase in βARK1 associated PI3K activity and protein important for the regulation of adrenergic receptors during this pathologic state.

### Example 7

### Direct Physical Interaction of PI3K with βARK1

The present inventors demonstrated that the HEAT domain of PI3K is an important interacting domain. In other proteins, such as the protein phosphatase 2A, importin-b and target of rapamycin (TOR), the HEAT domain is important for protein-protein interactions. The HEAT domain motif is named after proteins containing these repeats: Huntingtin Elongation Factor 3, A subunit of protein phosphatase 2A and TOR. To investigate whether there is a direct physical interaction between βARK1 and PI3K, we created PI3K mutants that contained only the HEAT domain (FLAG tagged) or had a deletion of the HEAT domain called PI3KΔHEAT (Hemagglutinin (HA) tagged) (FIG. 8A). FIG. 8A is a schematic representation of full length PI3 Kp110γ and mutants. ABR-adaptor binding region, RBD-ras binding domain, C2-similar to PLCδ, which is involved in Ca2+ dependent or independent phospholipid binding, HEAT-Helical domain thought to be involved in protein-protein interactions, HA-hemagglutinin tag, FLAG-flag peptide tag.

HEK 293 cells were transfected with plasmids containing the FLAG-HEAT and PI3KΔHEAT-HA cDNAs. FLAG-HEAT and PI3KΔHEAT proteins were immunoprecipitated from cell extracts using monoclonal FLAG and HA antibodies respectively, fractionated by SDS-PAGE and analyzed by immunoblotting with βARK1 monoclonal antibody. Following the addition of purified βARK1 protein to the immune complexes, the presence of βARK1 was assessed by immunoblotting for βARK1. βARK1 was found to associate with the HEAT protein and not to full-length PI3K lacking this domain (PI3KΔHEAT) (FIG. 8B). Levels of expression of HEAT and PI3KΔHEAT proteins were similar (FIG. 8B, lower panel). These data show that PI3K and βARK1 form a macromolecular complex within the cell.

To investigate whether there was a direct physical interaction between the HEAT domain of PI3K and βARK1, GST-HEAT fusion protein containing 535-732 amino acid residues of PI3Kγ was produced, which including the entire HEAT domain and flanking upstream 10 amino acids and downstream 7 amino acids. The GST-HEAT fusion protein was immobilized on sepharose beads and incubated with purified βARK1. Beads with bound GST alone or GST fusion proteins were incubated with purified βARK1. Beads were washed and bound material was run on SDS-PAGE and immunoblotted with βARKS monoclonal antibody. Purified βARK1 bound specifically to GST-HEAT immobilized beads and not to GST alone (FIG. 8C). No difference in the level of GST and GST-HEAT was found (FIG. 8C, lower panel).

The present inventors determined that the HEAT domain does not interact with Gβγ subunits of G-protein. Purified βARK1 and GST-HEAT fusion protein immobilized on sepharose beads were incubated with purified Gβγ. The beads were washed and recovered proteins were analyzed by SDS-PAGE followed by immunoblotting with a Gβ polyclonal antibody. While a strong association of Gβγ with βARK1 was found, no association of Gβγ with the HEAT domain was detected (FIG. 8D). Purified Gβγ was loaded as a positive control.

### Example 8

### HEAT Domain Displaces βARK1 Associated PI3K Activity in Living Cells

The present inventors determined that overexpression of the 197 amino acid HEAT domain of PI3K competed for endogenous PI3K binding to βARK1 in living cells. HEK 293 cells were co-transfected with plasmids containing the βARK1 cDNA (2 μg) and increasing concentrations of the HEAT domain cDNA (ranging from 0 to 6 μg). Cell lysates were immunoprecipitated using the βARK1 monoclonal antibody 72 hrs after transfection and βARK1 associated PI3K activity was assayed. A robust βARK1 associated PI3K activity was found in the absence of HEAT protein, but this association could be effectively competed away by increasing the concentration of HEAT cDNA (FIG. 9A). The maximal reduction of βARK1 associated PI3K activity occurred when cells were co-transfected with 4 μg of HEAT cDNA (FIG. 9B). Summary results of n=5 experiments. *, p<0.0005. The data was normalized to βARK1 associated PI3K activity in cells transfected with βARK1 only. Furthermore, FLAG tagged HEAT protein co-immunoprecipitated with βARK1 monoclonal antibodies in the co-transfected HEK 293 cells.

The absence of the HEAT domain in an otherwise intact PI3K molecule affected the association of βARK1 with endogenous PI3K in cells. HEK 293 cells were co-transfected with plasmids containing cDNAs for βARK1 (2 μg), βARK1 plus HEAT (4 μg), and βARK1 plus PI3KΔHEAT (4 μg). Cell lysates were immunoprecipitated with a βARK1 monoclonal antibody and assayed for the associated PI3K activity. Expression of PI3KΔHEAT had no effect on the endogenous βARK1/PI3K interaction, whereas overexpression of HEAT abolished this interaction (FIGS. 9C and 9D). FIG. 9D shows the summary results of n=3 experiments. *p<0.001. The data was normalized to βARK1 associated PI3K activity in cells transfected with βARK1 only.

### Example 9

### Overexpression of HEAT Blocks βARK1 Mediated Translocation of Endogenous PI3K

The present inventors demonstrated that overexpression of HEAT blocks βARK1 mediated translocation of PI3K to the membrane and to the β₂AR. HEK 293 cells were co-transfected with the βARK1 (2 pg), βARK1 and HEAT domain (4 μg) containing plasmids, and endogenous βARs were stimulated with 10 μM isoproterenol for 2 min. Cytosolic and membrane fractions were prepared and analyzed for βARK1 associated PI3K activity. Following isoproterenol stimulation, robust βARK1 associated PI3K activity was found in the membrane fraction (FIG. 10A). In contrast, overexpression of the HEAT protein effectively abolished the agonist-induced translocation of PI3K to the membrane by disrupting the βARK1/PI3K interaction. No change in βARK1 associated PI3K activity was found in cytosolic fractions after isoproterenol stimulation. βARK1 recruitment to the membrane was not inhibited in presence of HEAT protein. Membrane fractions were prepared from the HEK293 cells co-transfected with the βARK1 (2 μg), βARK1 and HEAT domain (4 μg) containing plasmids. As shown in FIG. 10B, overexpression of HEAT had no effect on the membrane recruitment of βARK1 following isoproterenol (10 μM) stimulation. These data show that the overexpression of HEAT protein interrupts the βARK1/PI3K interaction and does not affect agonist-dependent βARK1 translocation to the membrane.

Disruption of the βARK1/PI3K interaction prevented the recruitment of PI3K to activated βARs. HEK 293 cells were transfected with plasmids containing cDNAs for FLAG epitope tagged β₂AR (FLAG-β₂AR, 2 mg) or FLAG-β₂AR (2 μg) and HEAT domain (4 μpg), and assayed for E₂AR associated PI3K activity following stimulation with 10 μM isoproterenol. HEK 293 cells contain adequate levels of βARK1 to support agonist-induced translocation and receptor phosphorylation. FLAG tagged β₂ARs were immunoprecipitated from cell extracts and associated endogenous PI3K activity measured. FLAG-β₂AR associated PI3K activity was observed as early as 2 min after agonist stimulation, with a decline by 10 min in the cells transfected with β₂AR alone (FIG. 10C). In contrast, there was a marked decrease in FLAG-β₂AR associated PI3K activity in the presence of HEAT (FIG. 10C) indicating that overexpression of HEAT displaces PI3K from the βARK1 complex, thereby preventing its recruitment to the agonist-occupied receptor complex.

### Example 10

### Attenuation of β₂AR Sequestration by HEAT

Disruption of the endogenous βARK1/PI3K interaction by the HEAT protein attenuated β₂AR endocytosis. To test this, agonist-dependent (1 μM isoproterenol) sequestration over a time course of 0-30 min was studied by ¹²⁵I-Cyanopindolol binding in HEK 293 cells co-transfected with plasmids containing either the FLAG-β₂AR cDNA (▪), or FLAG-β₂AR and FLAG-HEAT cDNAs (□). A significant (>60%) attenuation in the rate of β₂AR sequestration occurred when the HEAT protein was overexpressed (FIG. 11A). Overexpression of HEAT protein was effective in attenuating the early processes of β₂AR sequestration since the initial phase (0-5 min) was significantly impaired. Receptor expression (fmol/mg of whole-cell protein) was: β₂AR+vector, 330±80; β₂AR+HEAT, 303±51. N=5, p<0.0001 versus β₂AR. Inset shows expression of HEAT protein.

Endocytosis of β₂AR-YFP in live cells was monitored for 10 min following isoproterenol (10 μM) stimulation in the absence or presence of HEAT protein co-expression using confocal microscopy. β₂AR internalization was followed in the same cell after agonist stimulation. Prior to agonist, the distribution of β₂AR-YFP was found distinctly at the plasma membrane (FIG. 11B, panel 1). Following agonist treatment, there was redistribution of the β₂AR-YFP into membrane puncta consistent with entry into clathrin coated pits (FIG. 11B, panel 2). With time, this was followed by the formation of cytoplasmic aggregates (FIG. 11B, panel 3), and then with the complete loss of membrane fluorescence (FIG. 11B, panels 3 and 7) (arrowheads). In marked contrast, co-expression with the HEAT protein completely prevented redistribution of β₂AR-YFP fluorescence into membrane puncta and blocked the formation of intracellular aggregates following isoproterenol stimulation (FIG. 11B, panels 6 and 8). In FIG. 11B, panels on the left represent cells transfected with the β₂AR-YFP alone (panels 1, 2 and 3 show the same cell monitored at 0, 5 and 10 min following stimulation. Panel 7 is an example of another cell after 10 min of isoproterenol). Panels on the right represent cell transfected with β₂AR-YFP and HEAT (panels 4,5 and 6 show the same cell monitored as above. Panel 8 is an example of another cell after 10 min of isoproterenol). In the absence of HEAT, isoproterenol caused the internalization β₂ARs as shown by the formation of distinct cytoplasmic aggregates (arrowheads) and complete loss of membrane fluorescence. In contrast, in the presence of HEAT protein, there is no redistribution of β₂AR-YFP following agonist stimulation indicating that the process of receptor endocytosis is completely inhibited.

Only those cells that contained the HEAT protein failed to undergo agonist-stimulated βAR internalization. Dual labeling experiments were performed after co-transfection with plasmids encoding HA tagged β₂AR (HA-β₂AR) and HEAT-GFP. After 10 min of 10 μM isoproterenol, cells were fixed with 4% paraformaldehyde, stained for HA-β₂AR receptor with Texas Red and HEAT was visualized by GFP fluorescence. Panel 1 shows two cells, one with only membrane distribution of HA-β₂AR, and another cell with complete redistribution of β₂ARs into aggregates (arrowheads). Cells that showed restricted distribution of Texas Red staining (HA-β₂AR expression) to the membrane (FIG. 11C, panel 1), also had GFP fluorescence indicating HEAT protein expression (FIG. 11C, panel 2). In contrast, cells that lacked GFP fluorescence (i.e. HEAT protein expression), showed marked agonist-induced β₂AR internalization (FIG. 11C, panel 1 and 2, arrowheads) as clearly seen in the overlay shown in panel 3 (FIG. 11C, panel 3, arrowheads).

### Example 11

### PI3K is Not Necessary for β-Arrestin Recruitment

Overexpression of HEAT neither inhibited β-arrestin2 recruitment to the receptor complex, phosphorylation of the receptor, nor downstream PI3K signaling. HEK 293 cells with stable expression of both β₂AR-HA and β-arrestin2-GFP proteins were transfected with the plasmid containing FLAG-HEAT cDNA and then split in separate dishes. Confocal microscopy was used to visualize fluorescence in fixed cells following stimulation with 10 μM isoproterenol for 10 min. Cells were fixed and stained with Texas Red. All cells showed β-arrestin2-GFP fluorescence whereas a smaller percentage showed Texas Red staining of the FLAG epitope (FIG. 12A, panels 1 and 2). In the absence of isoproterenol (FIG. 12A, panels 1 and 2) cells had a cytosolic distribution of HEAT as well as β-arrestin2-GFP. With isoproterenol, there was marked re-distribution of GFP fluorescence to the membrane indicating recruitment of β-arrestin to the membrane (FIG. 12A, panel 3). Importantly, cells that contained HEAT proteins did not affect the membrane recruitment of β-arrestin (FIG. 12A, panels 3 and 4, arrowheads), demonstrating that D-3 phosphoinositide molecules are not necessary for arrestin recruitment to activated receptors.

The overexpression of HEAT in cells did not alter other cellular processes involving downstream PI3K signaling. PKB activation was measured in HEK 293 cells transfected with plasmids containing cDNAs encoding the β₂AR or β₂AR plus HEAT (HEK 293 cells lack sufficient endogenous β₂AR for robust PKB activation), and stimulated with a variety of GPCR or growth factor agonists: MOCK, treated with ascorbic acid, IGF, treated with insulin growth factor (10 nM), Car, treated with carbachol (1 mM), ISO, treated with isoproterenol dissolved in ascorbic acid (10 μM). Agonist stimulation in the absence of HEAT resulted in a significant increase of phospho-PKB over mock treatment (FIG. 12B). Importantly, in HEAT transfected cells, robust PKB activation occurred in the presence of HEAT protein expression (lower panel in FIG. 12B) demonstrating no effect on cellular signaling downstream of PI3K.

### Example 12

### Role of D-3 Phosphatidylinositols in the Recruitment of Adaptin

The present inventors determined that βARK1-mediated localization of PI3K within the activated receptor complex is responsible for the generation of D-3 phosphatidylinositols necessary for the efficient recruitment of adaptin to the P₂AR complex. HEK 293 cells were transfected with FLAG-β₂AR or FLAG-β₂AR and HEAT plasmids and then stimulated with isoproterenol (10 μM) for 0, 2, 5, and 10 min. β₂AR was immunoprecipitated using the FLAG-epitope from cell extracts and immunoblotted with antibodies for the AP-2 adaptin and clathrin proteins. There was a significant increase in the association of AP-2 adaptin to the agonist-stimulated receptor within 2-5 min, which returned to basal levels by 10 min (FIG. 13A). In contrast, overexpression of the HEAT peptide completely abolished the recruitment of adaptin to the agonist-stimulated β₂AR complex (FIG. 13A). While the levels of clathrin that co-immunoprecipitated with the receptor showed only modest changes after agonist, this effect appeared to be attenuated in the presence of the HEAT peptide (FIG. 13A). Similar levels of adaptin, clathrin, P₂AR and HEAT were observed in the appropriately transfected cells (FIG. 13A, lower panel). IgG, represents heavy chain of the antibody. C, positive control for clathrin and AP-2 adaptin proteins.

Inhibition of PI3K activity affected the recruitment of adaptin to the receptor complex. FLAG-βAR was stably expressed in HEK 293 cells. Cells were treated with LY294002, an inhibitor of PI3K activity or DMSO as control for 15 min and then stimulated with isoproterenol for 0, 2, 5 and 10 min. FLAG-β₂AR was then immunoprecipitated using a FLAG epitope and lysates were immunoblotted for AP-2 adaptin protein. A significant increase in the association of adaptin protein with the receptor complex was observed at 5 min in the absence of LY294002, which was completely blocked with LY294002 pretreatment (FIGS. 13B and 13C, −LY, untreated cells, +LY cells treated with LY294002). FIG. 13C shows the summary results of densitometric analysis of adaptin recruitment to β₂AR in presence and absence of LY294002 (n=7). The data is represented as fold over basal. These data are consistent with the data showing that disruption of the βARK1/PI3K interaction with overexpression of HEAT leads to a loss in the receptor associated PI3K activity, a reduction in adaptin recruitment, and attenuation in receptor sequestration.

The present inventors demonstrated that the generation of D-3 Ptdlns phospholipids are important for the recruitment of AP-2 adaptor proteins to the agonist-occupied receptor complex. HEK 293 cells stably expressing FLAG-β₂AR were permeabilized with saponin and then incubated with increasing concentrations of the phosphorylated lipids, Ptdlns (4,5) P₂ and Ptdlns (3,4,5) P₃. Following stimulation with isoproterenol for 5 min, FLAG-β₂AR was immunoprecipitated and the immune complexes immunoblotted for the presence of adaptin. The efficiency of recruitment of adaptin to the receptor was significantly enhanced in the presence of Ptdlns (3,4,5) P₃ compared to a similar concentration of Ptdlns (4,5) P2 (FIGS. 13D and 13E) At high concentrations of Ptdlns (4,5) P2, the preferential effect of Ptdlns (3,4,5) P₃ was lost consistent with previous studies showing that the AP-2 adaptor protein has a higher affinity for Ptdlns (3,4,5) P₃ compared to other phosphoinositides. FIG. 13E shows the summary results of densitometric analysis of adaptin recruitment to β₂AR complex in presence of phosphatidyinositol-4,5-P₂ (PIP2) or phosphatidylinositol-3,4,5-P₃ (PIP3) (n=7). Data is represented as fold over basal.

The present inventors demonstrate a direct protein-protein interaction between PI3K and βARK1, and show that the region of the PI3K molecule that provides the necessary structure for this interaction is the HEAT domain. Moreover, the interaction between PI3K and βARK1 is not dependent on Gβγ and overexpression of HEAT peptide competitively displaced PI3K from the βARK1/PI3K complex leading to a loss in βARK1 associated PI3K activity. While overexpression of HEAT protein disrupted the βARK1/PI3K interaction, it did not inhibit the Gβγ mediated translocation of βARK1 to agonist-occupied receptors or alter other Gβγ dependent cellular processes. Finally, overexpression of the HEAT peptide markedly attenuated SEAR endocytosis in the early phase after agonist stimulation; impairing the local production of Ptdlns (3,4,5)P₃ lipid molecules within the agonist-occupied receptor complex, affected the recruitment of critical molecules necessary for efficient receptor endocytosis and the initiation/nucleation of the clathrin lattices at sites of endocytosis.

The expression of HEAT did not block β-arrestin recruitment to the receptor, nor did it impair the ability of βARK1 to phosphorylate activated receptors since β-arrestin is only recruited to GRK phosphorylated receptors. Even in the absence of receptor-associated PI3K activity, membrane Ptdlns (4,5) P2 was sufficient to recruit β-arrestin in an agonist-dependent manner.

The crystal structure of PI3Kγ shows the HEAT domain to be centrally positioned with a solvent exposed surface suitable for protein-protein interactions. It is therefore possible that other molecules containing a HEAT domain, such as PI3Kα, can potentially interact with βARK1. Interestingly, it has recently been shown that another PI3K, the class II PI3K C2a, interacts with clathrin and regulates clathrin-mediated membrane trafficking particularly in the process of vesicle uncoating. This suggests possible redundancy for the production of phosphoinositides within the receptor complex, a finding not surprising considering that receptor sequestration is a multi-step process, highly regulated by many molecules at different stages.

The present inventors demonstrated that the HEAT peptide displaced endogenous PI3K from βARK1 leading to impairment of PI3K translocation to the receptor following agonist stimulation. The loss in receptor associated PI3K activity impaired the ability of the agonist-occupied GPCR/PI3K complex to generate D-3 phospholipid molecules. The present inventors propose that the products of PI3K play a critical role in determining the dynamics of receptor endocytosis. Agonist-induced recruitment of class I PI3Ks by P3ARK1 to the receptor complex functioned to increase the production of D-3 phospholipid molecules, that in turn regulated the recruitment of AP-2 and the recruitment of cargo (i.e. receptor/β-arrestin complex) to clathrin coated pits on the membrane. The generation of Ptdlns (3,4,5) P₃ by PI3K within the activated receptor complex promoted more efficient recruitment of AP-2 and receptor sequestration. The rise in the local concentration of Ptdlns (3,4,5) P₃ within the receptor complex, which enhanced the recruitment of AP-2 to the complex, played a significant role in the initiation/nucleation of new clathrin-coated pits. The efficiency of clathrin coated pit formation will depend on the association of the various critical components that, in part, are regulated by their affinity to bind newly generated D-3 phospholipids.

### Example 13

### Attenuation of V2R Sequestration by HEAT

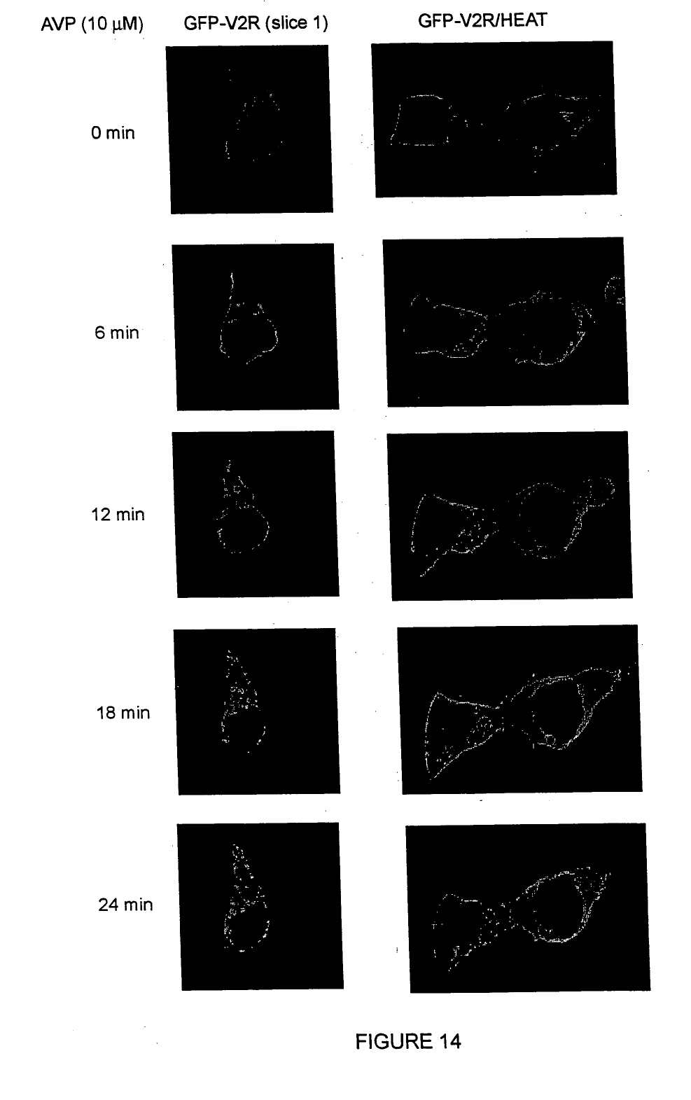

Disruption of the endogenous βARK1/PI3K interaction by the HEAT protein attenuated V2R endocytosis. Endocytosis of V2R-GFP in live cells was monitored for 20 min following AVP (10 μM) stimulation in the absence or presence of HEAT protein co-expression using confocal microscopy. HEK 293 cells were transfected with either GFP-V2R (2 μg)/pRK5 (2 μg) or GFP-V2R (2 μg)/HEAT (2 μg). V2R internalization was followed in the same cell after agonist stimulation. Prior to agonist, the distribution of V2R-GFP was found distinctly at the plasma membrane (FIG. 14, 0 min). Following agonist treatment, there was redistribution of the V2R-GFP into membrane puncta consistent with entry into clathrin coated pits. With time, this was followed by the formation of cytoplasmic aggregates, and then with the complete loss of membrane fluorescence (FIG. 14, 6-24 min). In marked contrast, co-expression with the HEAT protein completely prevented redistribution of V2R-GFP fluorescence into membrane puncta and blocked the formation of intracellular aggregates following agonist stimulation (FIG. 14, 6-24 min). In FIG. 14, panels on the left represent cells transfected with the V2R-GFP alone (panels show the same cell monitored at 0, 6, 12, 18, and 24 min following stimulation). Panels on the right represent cell transfected with V2R-GFP and HEAT (panels show the same cell monitored as above). In the absence of HEAT, agonist caused the internalization V2R5 as shown by the formation of distinct cytoplasmic aggregates and complete loss of membrane fluorescence. In contrast, in the presence of HEAT protein, there is no redistribution of V2R-GFP following agonist stimulation indicating that the process of receptor endocytosis is completely inhibited.

### Example 14

### Attenuation of AT1AR Sequestration by HEAT

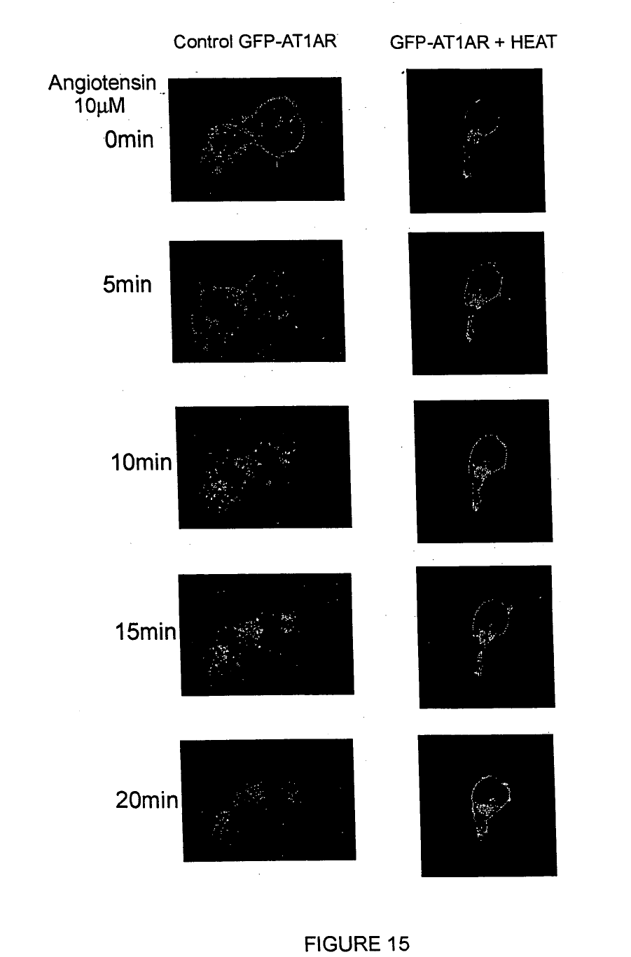

Disruption of the endogenous βARK1/PI3K interaction by the HEAT protein attenuated AT1AR endocytosis. Endocytosis of AT1AR-GFP in live cells was monitored for 20 min following angiotensin (10 μM) stimulation in the absence or presence of HEAT protein co-expression using confocal microscopy. HEK 293 cells were transfected with 2 μg Angiotensin II Type 1 Receptor, or with 2 pg Angiotensin II Type 1 Receptor and 2 μg HEAT, and stimulated for 20 minutes with 10 μM Angiotensin II. AT1AR internalization was followed in the same cell after agonist stimulation. Prior to agonist, the distribution of AT1AR-GFP was found distinctly at the plasma membrane (FIG. 15, 0 min). Following agonist treatment, there was redistribution of the AT1AR-GFP into membrane puncta consistent with entry into clathrin coated pits. With time, this was followed by the formation of cytoplasmic aggregates, and then with the complete loss of membrane fluorescence (FIG. 15, 5-20 min). In marked contrast, co-expression with the HEAT protein completely prevented redistribution of AT1AR-GFP fluorescence into membrane puncta and blocked the formation of intracellular aggregates following agonist stimulation (FIG. 15, 5-20 min). In FIG. 15, panels on the left represent cells transfected with the AT1AR-GFP alone (panels show the same cell monitored at 0, 5, 10, 15, and 20 min following stimulation). Panels on the right represent cell transfected with AT1AR-GFP and HEAT (panels show the same cell monitored as above). In the absence of HEAT, agonist caused the internalization AT1ARs as shown by the formation of distinct cytoplasmic aggregates and complete loss of membrane fluorescence. In contrast, in the presence of HEAT protein, there is no redistribution of AT1AR-GFP following agonist stimulation indicating that the process of receptor endocytosis is completely inhibited.

### Example 16

### PI3K Activity Associates with GRK2 (βARK1), but Not with GRK5

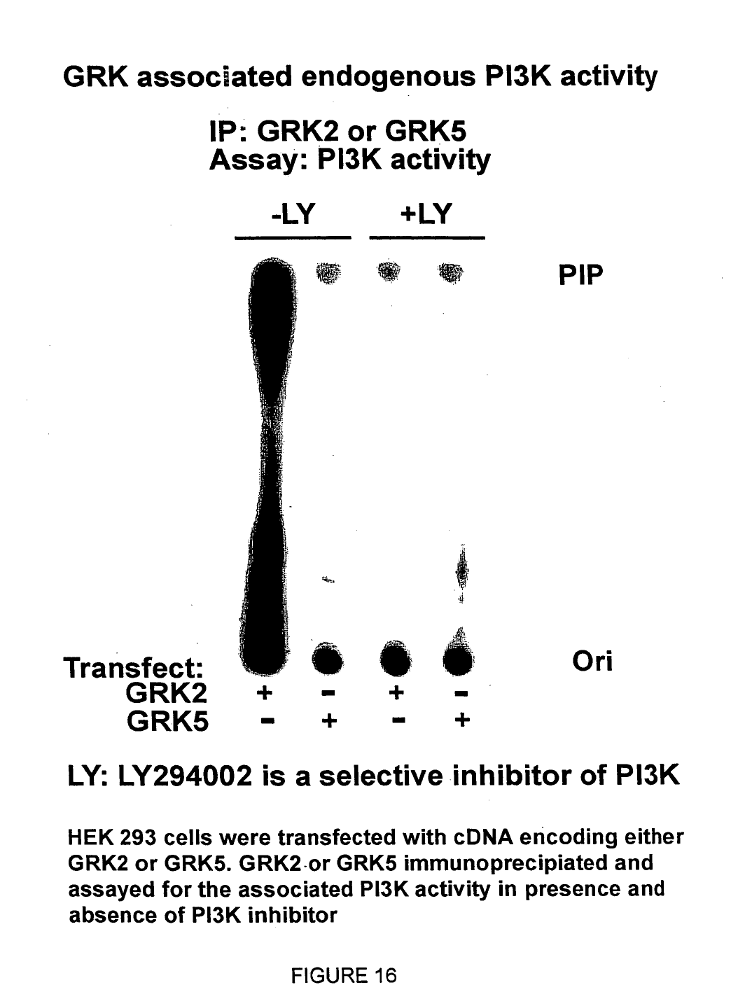

PI3K activity associates with GRK2 (βARK1), but not with GRK5, as illustrated in FIG. 16. HEK293 cells were transfected with cDNA encoding either GRK2 or GRK5. GRK2 or GRK5 was immunoprecipitated and assayed for the associated PI3K activity in the presence and absence in LY294002, a selective inhibitor of PI3K.

### Example 17

### Overexpression of Inactive PI3Kγ in Transgenic Mice Prevents Cardiac Dysfunction

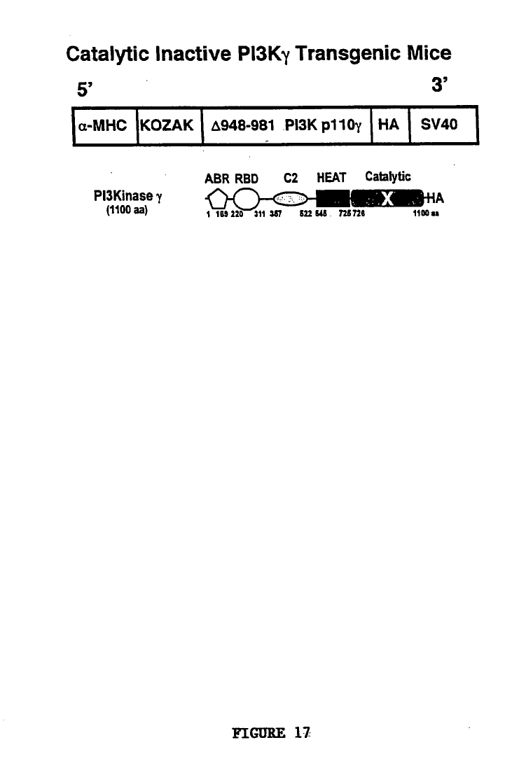

Catalytic inactive PI3Kg transgenic mice were constructed as shown in FIG. 17. The HotSHOT protocol was utilized to genotype the mice. Approximately 0.2 cm of tail was clipped from each mouse and added to 75 μl of alkaline lysis buffer (25 mM NaOH and 0.2 mM EDTA). Samples were boiled at 95° C. for 10 minutes then cooled to 4° C. 75 μl of neutralizing reagent (40 mM Tris HCl) was then added to each sample. 1 μl of this solution was added to 22 μl of ddH₂O along with 1 μl (15 mM) of each of 2 transgene-specific primers (Sigma-Genosys) and a PCR bead containing the taq polymerase, MgCl2 and dNTPs (Amersham Pharmacia Biotech). Samples were then placed in a Peltier Thermal Cycler (PTC-200—MJ Research) and cycled 30 times at [94° C. for 30 sec, 65° C. for 30 sec and 72° C. for 45 sec]. 10 p, of each sample was loaded onto a 2% agarose:TAE gel containing ethidium bromide. Bands were detected with a GelDoc 2000 (BioRad).

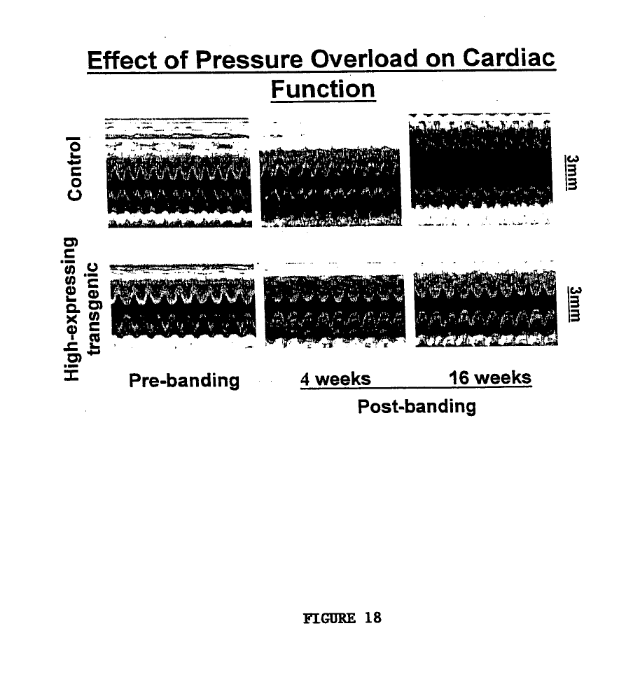

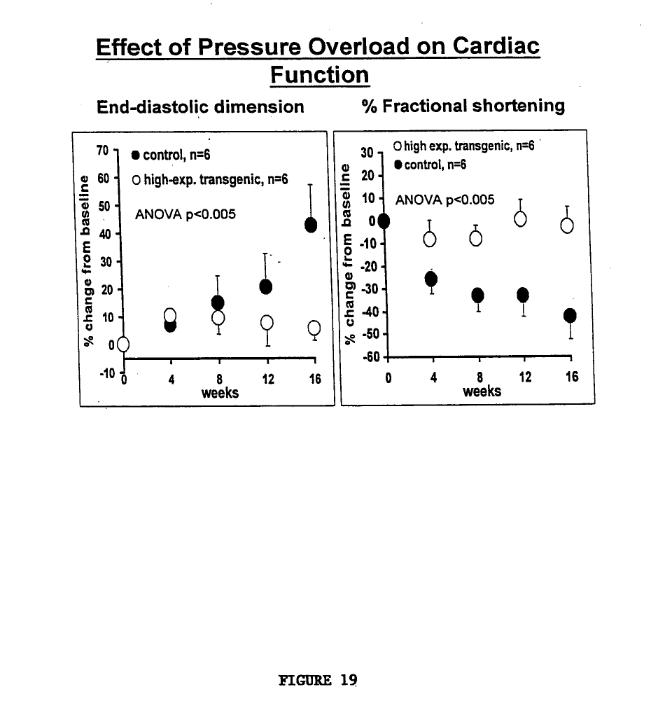

The effect of pressure overload on cardiac function was determined. As shown in FIGS. 18 and 19, the high-expressing transgenics had decreased change from baseline in end-diastolic dimension, as well as fractional shortening. Transthoracic 2D guided M-mode echocardiography was performed on conscious, unanesthetized mice at 3-4 months of age using an HDI 5000 echocardiograph (ATL, Bothell, Wash.). Miniosmotic pumps were inserted. Transgenics with 180-fold expression of the mutant protein and their wild-type littermates were anesthetized with a mixture of ketamine (10 mg/kg) and xylazine (0.5 mg/kg), and a small incision was made in the skin between the scapulae. A small pocket was created by spreading apart the subcutaneous connective tissue. Isoproterenol was dissolved in 0.002% ascorbic acid, and pumps were filled to deliver at a rate of 3 mg/kg/day over a period of 7 days. After insertion of the miniosmotic pump (model 2002; Alzet), the skin incision was closed with a 4.0 catgut suture. As controls, pumps that delivered vehicle (0.002% ascorbic acid) were implanted in mice. At the end of 7 days, hemodynamic evaluation was performed as described below. Hemodynamic evaluation in intact mice was performed. Mice were anesthetized with a mixture of ketamine (100 mg/kg) and xylazine (2.5 mg/kg) and, after endotracheal intubation, were connected to a rodent ventilator. After bilateral vagotomy, the left carotid artery was cannulated with a flame-stretched PE-50 catheter connected to a modified P-50 Statham transducer. A 1.4 French (0.46 mm) high-fidelity micromanometer catheter (Millar Instruments, Houston, Tex.) was inserted into the right carotid and advanced retrograde into the left ventricle. Hemodynamic measurements were recorded at baseline and 45 seconds after injection of incremental doses of isoproterenol (50, 500 and 1000 pg). Following the 1000 pg dose, the hearts were explanted, rinsed in cold PBS, and blotted dry. After weighing, isolated hearts were frozen in liquid nitrogen and stored at −80° C. until needed for biochemical studies.

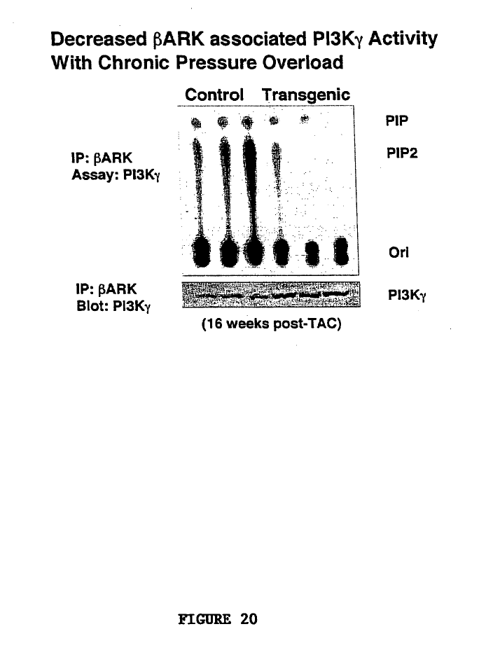

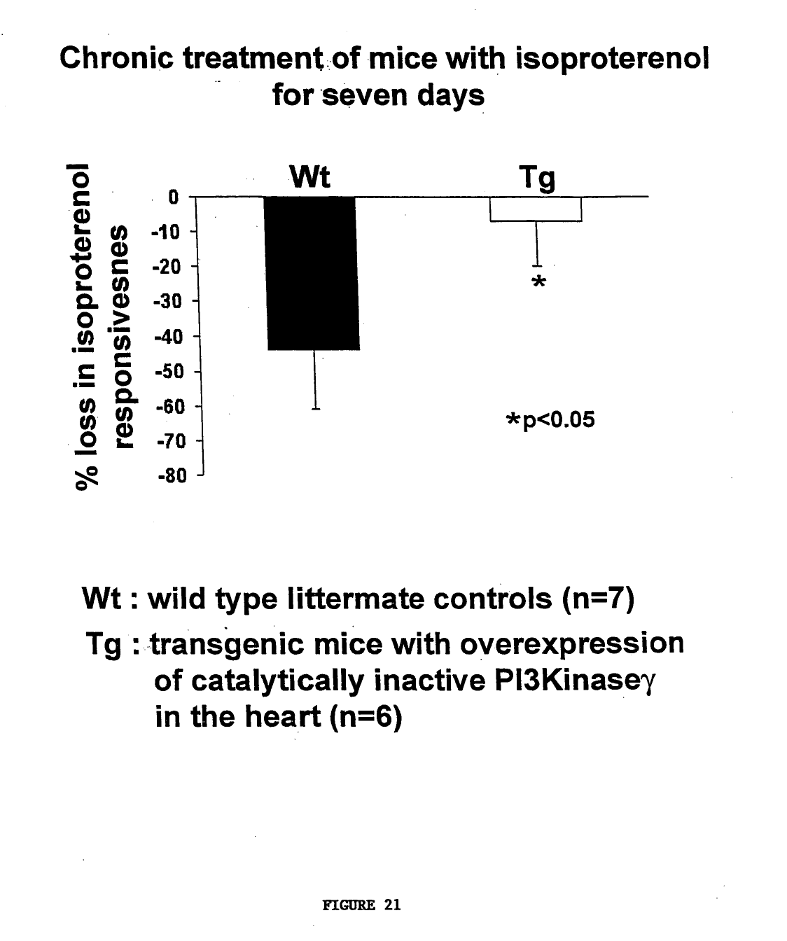

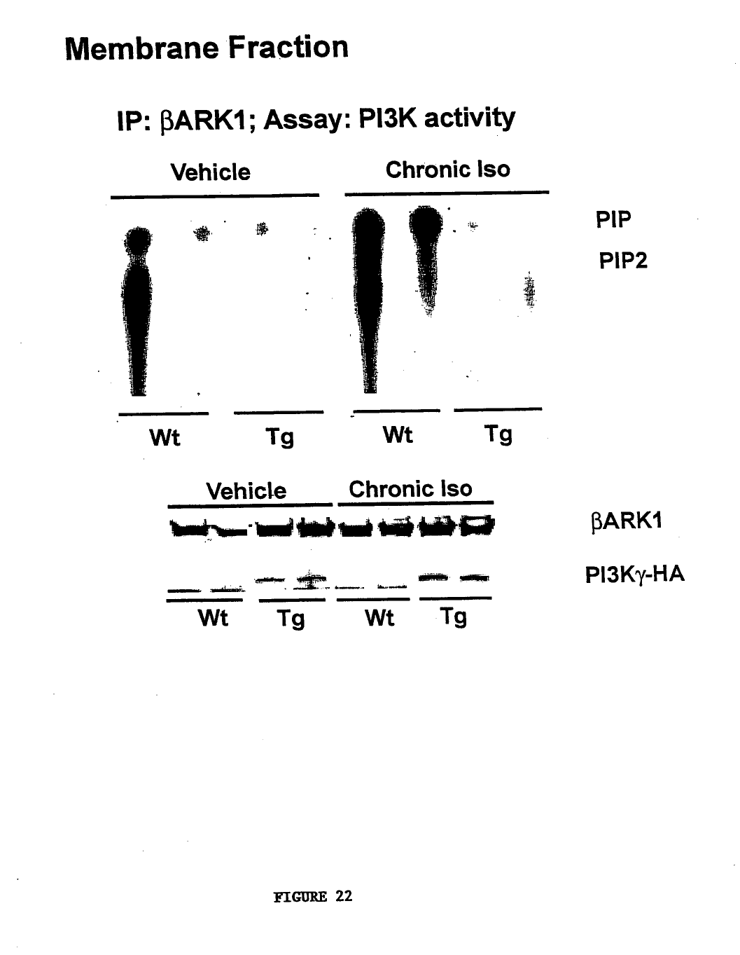

Decreased βARK1 associated PI3Kγ activity with chronic pressure overload was observed in transgenic mice overexpressing inactive PI3Kγ, as shown in FIG. 20. Transgenic mice also have a decreased % loss in isoproterenol responsiveness, as shown in FIG. 21. The transgenic mice lack PI3K activity associated with βARK1 immunoprecipitated from membrane fraction of hearts. A 2 mg sample of the extract was immunoprecipitated with antibodies to p110γ, p110β (Santa Cruz Biotechnology) or βARK1 and protein G-agarose. Sedimented beads were then washed once with 1 ml of lysis buffer, thrice with 1 ml of PBS (1×PBS, 1% NP-40 and 100 μM sodium orthovanadate), thrice with 1 ml of Tris/LiCl (100 mM Tris Cl (pH 7.4), 5 mM LiCl and 100 μM sodium orthovanadate) and twice with TNE (10 mM Tris Cl (pH 7.4), 150 mM NaCl, 5 mM EDTA and 100 μM sodium orthovanadate). Samples were then resuspended in 50 μl of TNE and 10 μl of 100 mM MgCl2 was added to each sample. The substrate, phosphatidylinositol (PI, Avanti), was prepared by drying 50 μl of the 10 mg/ml stock (PI in chloroform) in an eppendorf tube with a stream of air and adding 250 μl of TE (10 mM Tris Cl (pH 7.4) and 1 mM EDTA). The PI was then suspended by sonication in an ice bath for 3 minutes. 10 μl of the PI solution and 11 μl of ATP solution (440 mM cold ATP and 1 μCi/μl of [γ-³²P]ATP) were added to each sample. Samples were incubated at 22° C. for 10 minutes with continuous agitation. Reactions were terminated by adding 20 μl of 6N HCl. 160 μl of a 1:1 chloroform:methanol solution was added to each sample and samples were centrifuged at 14,000 RPM for 10 minutes, thus separating each sample into 3 phases. 40 μl of the lower (chloroform/lipid) phase was spotted onto 200-μm silica-coated flixi-TLC plates (Selecto-flexible, Fischer Scientific) precoated with 1% potassium oxalate and resolved chromatographically in 2 N glacial acetic acid:1-propanol (1:1.87). Autoradiography of dried plates was used to detect signals, and PIP was quantified by phosphorimaging. Values are expressed as fold increase/decrease relative to wild-type/control.

Immunoblotting and Detection: Cytosolic extracts were prepared as above and 70-140 μg of pure cytosolic extract was separated, transfered to a membrane, and then incubated overnight in blocking buffer containing the appropriate 1° antibody (p110γ, HA, PKB, GSK3β, p70-S6K, JNK, ERK, p38, p38β, βARK1 (Santa Cruz Biotechnologies) phospho-PKB (Ser-473), phospho-GSK3β (Ser-9) and phospho-p70-S6K (Thr-389 & 421/424) (Cell Signalling)) then washed thoroughly with TBS-t. Finally, the membrane was incubated in blocking buffer containing a 1:2000 dilution of the appropriate 2° (anti-rabbit, goat (p38β) or mouse (HA)) antibody (Amersham) conjugated with horseradish peroxidase. The membranes were then incubated in ECL (Amersham) and the chemiluminescent bands were detected by autoradiography. Bands were quantified with a densitometer (Bio-Rad) and values are expressed as fold increase/decrease relative to wild-type. Ligand Binding Assays: Membrane fractions were prepared from left ventricles via homogenization in 2 ml of lysis buffer (Buffer-A—25 mM Tris base (pH 7.5), 5 mM EDTA, 5 mM EGTA, 10 μg/ml Leupeptin, 20 mg/ml Aprotinin and 1 mM PMSF). Samples were centrifuged at 1000×g for 10 minutes and the supernatant is placed in a new tube and centrifuged at 48,000×g for 30 minutes. The pellets were resuspended in 1 ml of b-binding buffer (75 mM Tris-HCl, pH 7.4, 12.5 mM MgCl2, and 2 mM EDTA), centrifuged at 18,000 rpm for 30 minutes and again resuspended in 500 μl of β-binding buffer. Binding assays were performed on 25 μg of membrane protein using saturating amounts of ¹²⁵I-CYP (300 pM), a β-AR-specific ligand. Nonspecific binding was determined in the presence of 20 μM alprenolol. Reactions were conducted in either 250 or 500 μl of binding buffer at 37° C. for 1 hour and then terminated by vacuum filtration through glass-fiber filters. The β-AR-bound CYP trapped in the filter was then quantified with a gamma counter. All assays were performed in triplicate, and receptor density (fmol) is reported as picomoles of receptor per milligram of membrane protein.

While the invention has been described and illustrated herein by references to various specific material, procedures and examples, it is understood that the invention is not restricted to the particular material combinations of material, and procedures selected for that purpose. Numerous variations of such details can be implied as will be appreciated by those skilled in the art.

The following is a list of documents related to the above disclosure and particularly to the experimental procedures and discussions. The following documents, as well as any documents referenced in the foregoing text, should be considered as incorporated by reference in their entirety.
Attramadal, H., Arriza, J. L., Aoki, C., Dawson, T. M., Codina, J., Kwatra, M. M., Snyder, S. H., Caron, M. G. & Lefkowitz, R. J. (1992) J. Biol. Chem. 267,17882-17890
Barak, L. S., Oakley, R. H., Laporte, S. A. and Caron, M. G. (2001) Proc. Natl. Acad. Sci. USA 98, 93-98
Barak, L. S., Warabi, K., Feng, X., Caron, M. G. & Kwatra, M. M. (1999) J. Biol. Chem. 274, 7565-7569
Barak, L. S., Ferguson, S. S., Zhang, J. & Caron, M. G. (1997) J. Biol. Chem. 272, 27497-27500
Barak, L. S., Ferguson, S. S., Zhang, J., Martenson, C., Meyer, T. & Caron, M. G. (1997) Mol. Pharmacol. 51, 177-184
Barak, L. S., Menard, L., Ferguson, S. S., Colapietro, A. M. & Caron, M. G. (1995) Biochemistry 34, 15407-15414
Ferguson, S. S., Barak, L. S., Zhang, J. & Caron, M. G. (1996) Can. J. Physiol. Pharmacol. 74, 1095-1110
Ferguson, S. S., Menard, L., Barak, L. S., Koch, W. J., Colapietro, A. M. & Caron, M. G. (1995) J. Biol. Chem. 270, 24782-24789
Kim, K.-M., Valenzano, K. J., Robinson, S. R., Yao, W. D., Barak, L. S., Caron, M. G. (2001) J. Biol. Chem. 276: 37409-37414
Laporte, S. A., Oakley, R. H., Holt, J. A., Barak, L. S. & Caron, M. G. (2000) J. Biol. Chem. 275, 23120-23126
Laporte, S. A., Oakley, R. H., Zhang, J., Holt, J. A., Ferguson, S. S., Caron, M. G. & Barak, L. S. (1999) Proc. Natl. Acad. Sci. USA 96, 3712-3717
Leopoldt, D., Hanck, T., Exner, T., Maier, U., Wetzker, R., & Nürnberg, B. (1998) J. Biol. Chem. 273, 7024-7029
Menard, L., Ferguson, S. S., Zhang, J., Lin, F. T., Lefkowitz, R. J., Caron, M. G. & Barak, L. S. (1997) Mol. Pharmacol. 51, 800-808
Mhaouty-Kodja, S., Barak, L. S., Scheer, A., Abuin, L., Diviani, D., Caron, M. G. & Cotecchia, S. (1999) Mol. Pharmacol. 55, 339-347
Naga Prasad, S. V., Esposito, G., Mao, L., Koch, W. J., & Rockman, H. A. (2000) J. Biol. Chem. 275, 4693-4698
Naga Prasad, S. V., Barak, L. S., Rapacciuolo, A., Caron, M. G., & Rockman, H. A. (2001) J. Biol. Chem. 276,18953-18959
Neuwald, A. F. & Hirano T. (2000) Genome Research 10, 1445-1452
Oakley, R. H., Laporte, S. A., Holt, J. A., Barak, L. S., Caron, M. G. (2001). J. Biol. Chem. 276:19452-19460
Oakley, R. H., Laporte, S. A., Holt, J. A., Caron, M. G. & Barak, L. S. (2000) J. Biol. Chem. 275,17201-17210
Oakley, R. H., Laporte, S. A., Holt, J. A., Barak, L. S. & Caron, M. G. (1999) J. Biol. Chem. 274, 32248-32257
Wilbanks, A. M., Laporte, S. A., Barak, L. S. & Caron, M. G. (2002) Manuscript submitted.
Zhang, J., Barak, L. S., Anborgh, P. H., Laporte, S. A., Caron, M. G. & Ferguson, S. S. (1999) J. Biol. Chem. 274,10999-11006
Zhang, J., Barak, L. S., Winkler, K. E., Caron, M. G. & Ferguson, S. S. (1997) J. Biol. Chem. 272, 27005-27014

1. A method of screening compound(s) for modulating G protein-coupled receptor (GPCR) internalization, comprising the steps of:
(a) providing a cell comprising molecules involved in GPCR internalization, wherein the molecules involved in GPCR internalization comprise β-adrenergic receptor kinase 1 (βARK1), phosphoinositide 3-kinase (PI3K), GPCR, and arrestin, and wherein at least one of said molecules is detectably labeled;
(b) exposing the cell to the compound(s);
(c) identifying the location in the cell of the labeled molecule;
(d) comparing the location of the labeled molecule in the cell in the presence of the compound(s) to the location of the labeled molecule in the cell in the absence of the compound(s); and
(e) correlating a difference between (1) the location of the labeled molecule in the cell in the presence of the compound and (2) the location of the labeled molecule in the cell in the absence of the compound(s) to modulation of GPCR internalization.

2-23. (canceled)

24. A modified PI3K, wherein GPCR desensitization is altered when said modified PI3K is expressed in a cell.

25. The modified PI3K of claim 24, wherein the modified PI3K comprises a Huntingtin Elongation Factor 3, A subunit of protein phosphatase 2A and TOR (HEAT) domain.

26. The modified PI3K of claim 24, wherein the modified PI3K lacks a HEAT domain.

27. The modified PI3K of claim 24, wherein the modified PI3K lacks catalytic activity.

28. The modified PI3K of claim 24, comprising a polypeptide with the amino acid sequence of SEQ ID NO:2, 4, 6, 8, or 9.

29. The modified PI3K of claim 24, wherein the PI3K is a class IB PI3K.

30. The modified PI3K of claim 24, wherein the modified PI3K is a class IA PI3K.

31. The modified PI3K of claim 24, wherein the ability of a GPCR to bind adaptin is altered when said modified PI3K is expressed in a cell.

32. The modified PI3K of claim 24, wherein the modified PI3K is PI3Kγ.

33. The modified PI3K of claim 24, wherein cellular phosphatidylinositol 3,4,5-triphosphate (Ptdlns (3,4,5) P₃) levels are altered when said modified PI3K is expressed in a cell.

34. The modified PI3K of claim 24, wherein the ability of wild-type PI3K to bind βARK1 is altered.

35. The modified PI3K of claim 25, wherein the modified PI3K has the ability to bind βARK1.

36. The modified PI3K of claim 26, wherein the modified PI3K lacks the ability to bind βARK1.

37. The modified PI3K of claim 24, wherein the modified PI3K is conjugated to a detectable molecule.

38. A modified βARK1 which lacks the ability to bind PI3K.

39. An isolated nucleic acid sequence encoding the modified PI3K of claim 24.

40. The isolated nucleic acid of claim 39, comprising a nucleic acid with the nucleic acid sequence of SEQ ID NO:1.

41. An expression vector comprising the nucleic acid of claim 40 operably linked to an expression control sequence.

42. A host cell comprising the expression vector of claim 41.

43. A host cell comprising the nucleic acid of claim 40 integrated in its genome.

44. A non-human transgenic animal which expresses a modified PI3K of claim 24.

45-46. (canceled)

47. A compound identified by the method of claim 1.

48. A kit for detecting a βARK1/PI3K complex in a biological sample comprising an antibody which recognizes and binds to the βARK1/PI3K complex and reagents which detect the antibody that binds to the βARK1/PI3K complex.

49. An isolated immunoglobulin which binds to a βARK1/PI3K complex.

50. The immunoglobulin of claim 49, wherein the immunoglobulin is a monoclonal antibody, a chimeric antibody, a human antibody, a bispecific antibody, a humanized antibody, a primatized antibody, or an antibody fragment.

51. (canceled)

52. A method of altering GPCR internalization, comprising providing an effective amount of LY294002 or wortmannin.

53. (canceled)

54. A method of preventing and/or treating a disease associated with GPCR activity in mammals, comprising providing to an animal a therapeutically effective amount of a compound according to claim 47 and a pharmaceutically acceptable carrier.

55. A method of preventing and/or treating a disease associated with PI3K activity in mammals, comprising providing to an animal a therapeutically effective amount of a compound according to claim 47 and a pharmaceutically acceptable carrier.

56. The method of claim 54, wherein the treated disease is a cardiovascular disease, heart failure, asthma, nephrogenic diabetes insipidus, or hypertension.

57. A method of preventing and/or treating a disease associated with GPCR activity in mammals, comprising administering to a mammal an amount of the isolated nucleic acid of claim 39 sufficient to reduce or alleviate symptoms of said disease.

58. The method of claim 57, wherein the treated disease is a cardiovascular disease, heart failure, asthma, nephrogenic diabetes insipidus, or hypertension.
